SEQUENCE LISTING (4 sequences)

SEQ ID NO: 1 - DNA, 6531 bp, Artificial
  pACYCDuet-1-Phe
ggggaattgtgagcggataacaattcccctgtagaaataattttgtttaactttaataaggagatataccatggatggcttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataagtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctatgagcagcgtagatttttaactggatccccgggaattcagaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattggtttactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 2 - DNA, 1568 bp, Artificial
  Firefly luciferase
atgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattggttta

SEQ ID NO: 3 - DNA, 936 bp, Artificial
  Renellar luciferase
atggcttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 4 - DNA, 6 bp, Artificial
  sequence for detection
ttttaa

(19) United States
(12) Patent Application Publication
(10) Pub. No.: US 2016/0060235 A1
(43) Pub. Date: Mar. 3, 2016

(54) Therapeutic Agent for Type 2 Diabetes

(71) Applicants: NATIONAL UNIVERSITY CORPORATION KUMAMOTO UNIVERSITY, Kumamoto (JP); KUMAMOTO HEALTH SCIENCE UNIVERSITY, Kumamoto (JP)

(72) Inventors: Kazuhito Tomizawa, Kumamoto (JP); Fanyan Wei, Kumamoto (JP); Kengo Inoue, Shizuoka (JP); Tadashi Okawara, Kumamoto (JP)

(21) Appl. No.: 14/780,052

(22) PCT Filed: Mar. 28, 2014
(86) PCT No.: PCT/JP2014/001853

§ 371 Date: Sep. 25, 2015

(30) Foreign Application Priority Data
Mar. 29, 2013 (JP) 2013-072391

(51) Int. Cl. C07D 295/108; C07D 211/58

(52) U.S. Cl. CPC C07D 295/108; C07D 211/58

(57) ABSTRACT

An object of the present invention is to provide a novel therapeutic agent for a patient with type 2 diabetes, a cause of which is the abnormal synthesis of insulin attributed to the abnormal modification of tRNA^{Lys} (UUU) in pancreatic β cells having Cdkal1 gene mutation. The present inventors have used (1) a screening system using E. coli in which correct translation into luciferase requires frameshift resulting from mistranslation during protein translation, (2) a screening system using the pancreatic islet of Langerhans isolated from a pancreatic β cell-specific Cdkal1-deficient mouse, and (3) a screening system using a pancreatic β cell-specific Cdkal1-deficient mouse, and found that a compound represented by any of the following formulas (I) to (III) can serve as a therapeutic agent for a patient with type 2 diabetes with Cdkal1 gene mutation resulting in the reduced ability to secrete insulin.

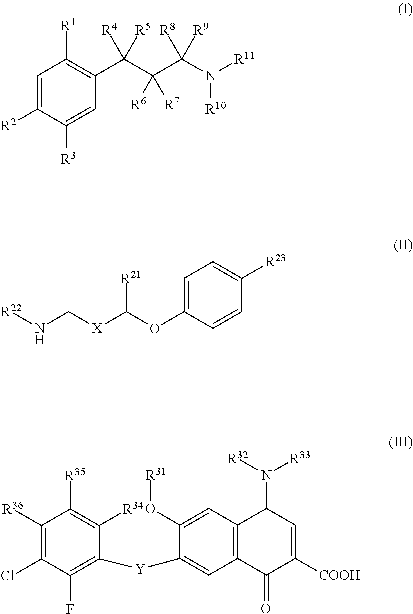

### TECHNICAL FIELD

The present invention relates to a therapeutic agent for type 2 diabetes patient with Cdkal1 gene mutation resulting in the reduced ability to secrete insulin.

### BACKGROUND ART

Type 2 diabetes is one of the most common lifestyle-related diseases in middle-aged or older adults. Its prevalence is increasing in many countries including Japan. Most of type 2 diabetes patients are considered to have developed this disease due to environmental factors (obesity, a lack of exercise, high-fat diets, etc.) in addition to genetic factors. Thus, people who have been found likely to suffer from diabetes before its development by the preclinical diagnosis of genetic factors related to diabetes can be prevented from developing diabetes by increasing awareness of diets and exercise.

In recent years, correlation analysis over the whole genome permits detection of genetic mutation, called single-nucleotide polymorphism (SNP), which is detected at high frequency in the sequenced genome. Some SNPs have already been reported as to type 2 diabetes (patent documents 1 and 2 and non-patent documents 1 to 4). Specific SNP mutation located in the Cdkal1 (Cdk5 regulator subunit associated protein 1-like 1) gene is known, as gene mutation that increases the risk of development of type 2 diabetes, to significantly correlate with a decline in insulin secretion and the development of type 2 diabetes (non-patent documents 5 to 8).

Type 2 diabetes in westerners becomes less responsive to insulin due to marked obesity and developed insulin resistance, whereas type 2 diabetes in Asians including Japanese causes mild obesity, but decreases the ability itself of pancreatic β cells to secrete insulin. This has implied the mechanism underlying the development of type 2 diabetes differs depending on race. SNPs in the Cdkal1 gene are related to type 2 diabetes, irrespective of race. However, as a result of comparing the ratio of risk alleles of SNPs in the Cdkal1 gene between races, the Asian race carries the risk alleles more than the European race (non-patent documents 9 to 10). This has suggested the possibility that the Cdkal1 gene mutation is involved in the development of Japanese diabetes.

Insulin is first synthesized as preproinsulin in pancreatic β cells. The preproinsulin consists of a signal peptide, a B chain, a C-peptide, and an A chain. This preproinsulin is folded at two disulfide bonds, and the signal peptide is cleaved to form proinsulin. The C-peptide moiety is further cleaved from the proinsulin, and the remaining portion constitutes insulin. A lysine residue is present at this cleavage site between the C-peptide and the A chain.

It has been reported that: Cdkal1 specifically recognizes tRNA^{Lys} (UUU), which is tRNA corresponding to lysine codons AAA and AAG, to thiomethylate adenine at position 37 near the anticodon; and the thiomethylation of tRNA^{Lys} (UUU) by Cdkal1 prevents the mistranslation of the AAA and AAG codons (non-patent document 11). Pancreatic β cells deficient in Cdkal1 therefore produce abnormal insulin incapable of processing due to the mistranslation of the lysine residue in proinsulin. These facts have implied that the abnormal synthesis of insulin attributed to the abnormal modification of tRNA^{Lys} (UUU) contributes to increase in the risk of development of type 2 diabetes.

Currently commercially available sulfonylurea antidiabetic agents act directly on the potassium channel on pancreatic β cells so that the β cells are forced to secrete insulin. However, these agents might cause hypoglycemia when administered during the fasting state, and are known to cause a secondary failure phenomenon in which the effects of the agents gradually become poor as a result of long-term administration (non-patent documents 12 to 13). GLP1-mediated antidiabetic agents indirectly accelerate insulin secretion by acting on receptors present on the surface of pancreatic β cells. However, their effects and safety of long-term administration have not been established yet. In addition, these agents are highly expensive and place a significant burden on patients. Antidiabetic agents typified by biguanides, etc., improve the symptoms of diabetes mainly by assisting in the antihyperglycemic effect of insulin. However, these agents do not radically improve the function of secreting insulin by pancreatic β cells (non-patent document 14).

### PRIOR ART DOCUMENTS

### Patent Documents

Patent Document 1: Japanese unexamined Patent Application Publication No. 2006-115811
Patent Document 2: Japanese unexamined Patent Application Publication No. 2009-219458

### Non-Patent Documents

Non-patent Document 1: Steinthorsdottir V., et al., Variant in CDKAL1 influences insulin response and risk of type 2 diabetes. Nat. Genet. 2007, 39 (6), p. 770-775.
Non-patent Document 2: Saxena R., et al., Genome-wide association analysis identifies loci for type 2 diabetes and triglyceride levels. Science. 2007, 316 (5829), p. 1331-1336.
Non-patent Document 3: Scott L. J., et al., A genome-wide association study of type 2 diabetes in Finns detects multiple susceptibility variants. Science. 2007, 316 (5829), p. 1341-1345.
Non-patent Document 4: Zeggini E., et al., Replication of genome-wide association signals in UK samples reveals risk loci for type 2 diabetes. Science. 2007, 316 (5829), p. 1336-1341.
Non-patent Document 5: Dehwah M. A., et al., CDKAL1 and type 2 diabetes: a global meta-analysis. Genet. Mol. Res. 2010, 9 (2), p. 1109-1120.
Non-patent Document 6: Groenewoud M. J., et al., Variants of CDKAL1 and IGF2BP2 affect first-phase insulin secretion during hyperglycaemic clamps. Diabetologia. 2008, 51 (9), p. 1659-1663.
Non-patent Document 7: Stancakova A., et al., Association of 18 confirmed susceptibility loci for type 2 diabetes with indices of insulin release, proinsulin conversion, and insulin sensitivity in 5,327 nondiabetic Finnish men. Diabetes. 2009, 58 (9), p. 2129-2136.
Non-patent Document 8: Ruchat S. M., et al., Association between insulin secretion, insulin sensitivity and type 2 diabetes susceptibility variants identified in genome-wide association studies. Acta Diabetol. 2009, 46 (3), p. 217-226.
Non-patent Document 9: Wei F. Y., et al., Functional loss of Cdkal1, a novel tRNA modification enzyme, causes the development of type 2 diabetes. Endocr. J. 2011, 58 (10), p. 819-25.
Non-patent Document 10: Kazuhito Tomizawa, Biological functions of Cdkal1, a Japanese race diabetes-related gene, Journal of Clinical and Experimental Medicine, 2012, 240 (4), p. 318-319.
Non-patent Document 11: Wei F. Y., et al., Deficit of tRNALys modification by Cdkal1 causes the development of type 2 diabetes in mice. J. Clin. Invest. 2011, 121 (9), p. 3598-3608.
Non-patent Document 12: Hiroko Kanno, et al., Primary failure/secondary failure of sulfonylurea drugs and measures against the failures, Japanese Journal of Clinical Medicine, 2002, Suppl (4), p. 719-723.
Non-patent Document 13: Ishii H., et al., Glucose-incretin interaction revisited. Endocr. J. 2011, 58 (7) p. 519-525.
Non-patent Document 14: Nichols G. A., et al., Treatment escalation and rise in HbA1c following successful initial metformin therapy. Diabetes Care, 2006, 29, p. 504-509.

### SUMMARY OF THE INVENTION

### Object to be Solved by the Invention

An object of the present invention is to provide a novel therapeutic agent for a patient with type 2 diabetes, a cause of which is the abnormal synthesis of insulin attributed to the abnormal modification of tRNA^{Lys} (UUU) in pancreatic β cells having Cdkal1 gene mutation.

### Means to Solve the Object

A therapeutic agent for diabetes that acts on a decline in insulin secretion caused by abnormal protein translation attributed to gene mutation has not been reported yet. The present inventors have first constructed a screening system using E. coli in which correct translation into luciferase requires frameshift resulting from mistranslation during protein translation. The present inventors have administered each low-molecular compound to this E. coli to reduce the luminescence intensity of luciferase. Specifically, the present inventors have screened for a low-molecular compound that improves the accuracy of translation (primary screening). Next, the present inventors have screened for a compound that increases the ability of pancreatic β cells to secrete insulin, by administering a compound found positive in the primary screening to the pancreatic islet of Langerhans isolated from a pancreatic β cell-specific Cdkal1-deficient mouse (secondary screening). The present inventors have further studied glucose tolerance by administering a compound found positive in the secondary screening to a pancreatic β cell-specific Cdkal1-deficient mouse, then injecting a glucose solution thereto, and measuring the blood glucose level and the serum insulin concentration over time (tertiary screening). The present inventors have found by these screening operations that the compound of the present invention can serve as a therapeutic agent that improves reduction in the ability to secrete insulin, a cause of which is Cdkal1 gene mutation. The present invention has been completed on the basis of these findings.

Specifically, the present invention relates to:

(1) a therapeutic agent for type 2 diabetes comprising one or more compounds selected from the group consisting of a compound represented by the following formula (I):

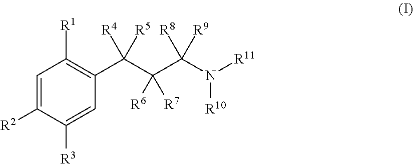

[wherein

R¹ represents any one group selected from hydrogen, a substituted or unsubstituted linear or branched alkyl group having 1 to 4 carbon atoms, a substituted or unsubstituted linear or branched alkenyl group having 2 to 4 carbon atoms, and a substituted or unsubstituted linear or branched alkynyl group having 2 to 4 carbon atoms,

R² represents any one group selected from hydrogen, halogen, a hydroxyl group, a substituted or unsubstituted linear or branched alkyl group having 1 to 10 carbon atoms, a substituted or unsubstituted linear or branched alkenyl group having 2 to 10 carbon atoms, a substituted or unsubstituted linear or branched alkynyl group having 2 to 10 carbon atoms, a substituted or unsubstituted alicyclic group having 3 to 10 carbon atoms, a substituted or unsubstituted aryl group having 6 to 10 carbon atoms, a substituted or unsubstituted alkoxy group having 1 to 10 carbon atoms, a substituted or unsubstituted acyl group having 1 to 11 carbon atoms, a carboxyl group and an ester derivative or an amide derivative thereof, a substituted or unsubstituted sulfonyl group having 1 to 10 carbon atoms, and a substituted or unsubstituted sulfide group having 1 to 10 carbon atoms,

R³ represents any one group selected from hydrogen, halogen, a substituted or unsubstituted linear or branched alkyl group having 1 to 4 carbon atoms, a substituted or unsubstituted linear or branched alkenyl group having 2 to 4 carbon atoms, a substituted or unsubstituted linear or branched alkynyl group having 2 to 4 carbon atoms, a substituted or unsubstituted aryl group having 6 to 10 carbon atoms, and a substituted or unsubstituted linear or branched alkoxy group having 1 to 4 carbon atoms,

R⁴ represents any one group selected from hydrogen, a substituted or unsubstituted aryl group having 6 to 10 carbon atoms, and a substituted or unsubstituted aryloxy group having 6 to 10 carbon atoms, or optionally forms a carbonyl group together with R⁵ and carbon bonded thereto,

R⁵ represents hydrogen or optionally forms a carbonyl group together with R⁴ and carbon bonded thereto,

R⁶ represents any one group selected from hydrogen, halogen, a substituted or unsubstituted linear or branched alkyl group having 1 to 6 carbon atoms, a substituted or unsubstituted linear or branched alkenyl group having 2 to 6 carbon atoms, a substituted or unsubstituted linear or branched alkynyl group having 2 to 6 carbon atoms, a substituted or unsubstituted alicyclic group having 3 to 6 carbon atoms, a substituted or unsubstituted linear or branched alkyloxycarbonyl group having 1 to 4 carbon atoms, a substituted or unsubstituted linear or branched alkenyloxycarbonyl group having 2 to 4 carbon atoms, a substituted or unsubstituted linear or branched alkynyloxycarbonyl group having 2 to 4 carbon atoms, a substituted or unsubstituted aryl group having 6 to 10 carbon atoms, a carboxyl group and an ester derivative or an amide derivative thereof, a cyano group, and an amino group,

R⁷ represents any one group selected from hydrogen, a substituted or unsubstituted linear or branched alkyl group having 1 to 4 carbon atoms, a substituted or unsubstituted linear or branched alkenyl group having 2 to 4 carbon atoms, and a substituted or unsubstituted linear or branched alkynyl group having 2 to 4 carbon atoms,

R⁸ represents any one group selected from hydrogen, halogen, a substituted or unsubstituted linear or branched alkyl group having 1 to 6 carbon atoms, a substituted or unsubstituted linear or branched alkenyl group having 2 to 6 carbon atoms, a substituted or unsubstituted linear or branched alkynyl group having 2 to 6 carbon atoms, a substituted or unsubstituted alicyclic group having 3 to 6 carbon atoms, a substituted or unsubstituted linear or branched alkyloxycarbonyl group having 1 to 4 carbon atoms, a substituted or unsubstituted linear or branched alkenyloxycarbonyl group having 2 to 4 carbon atoms, a substituted or unsubstituted linear or branched alkynyloxycarbonyl group having 2 to 4 carbon atoms, a substituted or unsubstituted aryl group having 6 to 10 carbon atoms, a carboxyl group and an ester derivative or an amide derivative thereof, a cyano group, and an amino group, or optionally forms a carbonyl group together with R⁹ and carbon bonded thereto,

R⁹ represents hydrogen, or optionally forms a carbonyl group together with R⁸ and carbon bonded thereto,

R¹⁰ represents any one group selected from hydrogen, a substituted or unsubstituted linear or branched alkyl group having 1 to 6 carbon atoms, a substituted or unsubstituted linear or branched alkenyl group having 2 to 6 carbon atoms, a substituted or unsubstituted linear or branched alkynyl group having 2 to 6 carbon atoms, a substituted or unsubstituted alicyclic group having 3 to 6 carbon atoms, a substituted or unsubstituted linear or branched alkyloxycarbonyl group having 1 to 6 carbon atoms, a substituted or unsubstituted linear or branched alkenyloxycarbonyl group having 2 to 6 carbon atoms, a substituted or unsubstituted linear or branched alkynyloxycarbonyl group having 2 to 6 carbon atoms, a substituted or unsubstituted aryl group having 5 to 10 carbon atoms, a carboxyl group and an ester derivative or an amide derivative thereof, a cyano group, and an amino group,

R¹¹ represents any one group selected from hydrogen, a substituted or unsubstituted linear or branched alkyl group having 1 to 6 carbon atoms, a substituted or unsubstituted linear or branched alkenyl group having 2 to 6 carbon atoms, a substituted or unsubstituted linear or branched alkynyl group having 2 to 6 carbon atoms, and a substituted or unsubstituted alicyclic group having 3 to 6 carbon atoms, and

R¹⁰ and R¹¹ optionally constitute a substituted or unsubstituted nitrogen-containing heterocyclic ring together with the nitrogen atom bonded thereto],

a compound represented by the formula (II):

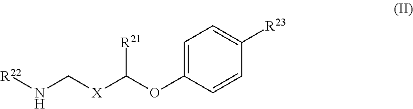

[wherein

R²¹ represents any one group selected from hydrogen, a substituted or unsubstituted linear or branched aryl group having 6 to 10 carbon atoms, and a substituted or unsubstituted linear or branched deuterated aryl group having 6 to 10 carbon atoms,

R²² represents any one group selected from a substituted or unsubstituted linear or branched alkyl group having 1 to 4 carbon atoms, a substituted or unsubstituted linear or branched deuterated alkyl group having 1 to 4 carbon atoms, a substituted or unsubstituted linear or branched alkenyl group having 2 to 4 carbon atoms, a substituted or unsubstituted linear or branched deuterated alkenyl group having 2 to 4 carbon atoms, a substituted or unsubstituted linear or branched alkynyl group having 2 to 4 carbon atoms, and a substituted or unsubstituted linear or branched deuterated alkynyl group having 2 to 4 carbon atoms,

R²³ represents any one group selected from hydrogen, a substituted or unsubstituted linear or branched alkyl group having 1 to 4 carbon atoms, a substituted or unsubstituted linear or branched deuterated alkyl group having 1 to 4 carbon atoms, a substituted or unsubstituted linear or branched alkenyl group having 2 to 4 carbon atoms, a substituted or unsubstituted linear or branched deuterated alkenyl group having 2 to 4 carbon atoms, a substituted or unsubstituted linear or branched alkynyl group having 2 to 4 carbon atoms, and a substituted or unsubstituted linear or branched deuterated alkynyl group having 2 to 4 carbon atoms, and

X represents methylene or deuterated methylene], and a compound represented by the formula (III):

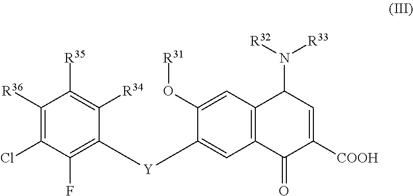

[wherein

R³¹ represents any one group selected from a substituted or unsubstituted linear or branched alkyl group having 1 to 4 carbon atoms, a substituted or unsubstituted linear or branched deuterated alkyl group having 1 to 4 carbon atoms, a substituted or unsubstituted linear or branched alkenyl group having 2 to 4 carbon atoms, a substituted or unsubstituted linear or branched deuterated alkenyl group having 2 to 4 carbon atoms, a substituted or unsubstituted linear or branched alkynyl group having 2 to 4 carbon atoms, a substituted or unsubstituted linear or branched deuterated alkynyl group having 2 to 4 carbon atoms, a substituted or unsubstituted heterocyclic group having a 5- or 6-membered ring, a substituted or unsubstituted aromatic group having 6 to 10 carbon atoms, a substituted or unsubstituted nitrogen-containing aromatic group having 2 to 10 carbon atoms, a sulfonic acid group, and a sulfonyl group,

R³² represents any one group selected from hydrogen, a hydroxymethyl group, and a hydroxymethyl group in which hydrogen on carbon is substituted by deuterium,

R³³ represents any one group selected from a substituted or unsubstituted linear or branched alkyl group having 1 to 6 carbon atoms, a substituted or unsubstituted linear or branched deuterated alkyl group having 1 to 6 carbon atoms, a substituted or unsubstituted linear or branched alkenyl group having 2 to 6 carbon atoms, a substituted or unsubstituted linear or branched deuterated alkenyl group having 2 to 6 carbon atoms, a substituted or unsubstituted linear or branched alkynyl group having 2 to 6 carbon atoms, a substituted or unsubstituted linear or branched deuterated alkynyl group having 2 to 6 carbon atoms, and a substituted or unsubstituted alicyclic group having 3 to 6 carbon atoms,

R³⁴ represents hydrogen, a hydroxymethyl group, or a hydroxymethyl group in which hydrogen on carbon is substituted by deuterium,

R³⁵ represents any one group selected from hydrogen, a hydroxyl group, a substituted or unsubstituted nitrogen-containing heterocyclic group having a 5- or 6-membered ring, and a substituted or unsubstituted nitrogen-containing aromatic group having 2 to 10 carbon atoms,

R³⁶ represents any one group selected from hydrogen, a hydroxyl group, and halogen, and

Y represents any one selected from methylene, deuterated methylene, and hydroxymethylene]

and pharmaceutically acceptable salts thereof.

The present invention also relates to:

(2) the therapeutic agent for type 2 diabetes according to (1), wherein the compound is selected from the compounds represented by the following formulas (I-1) to (I-6), (II-1), (II-2), and (III-1),

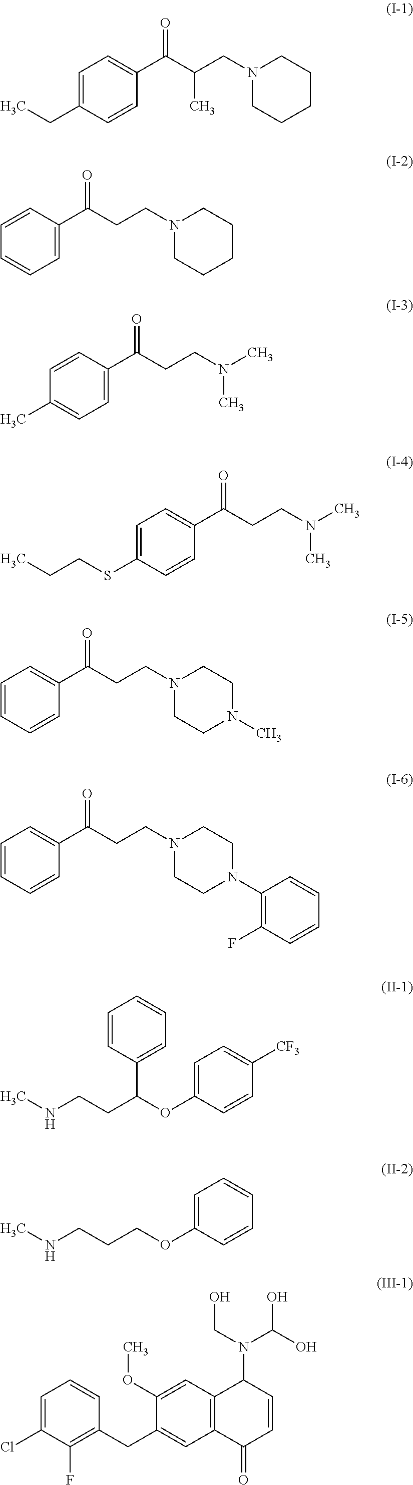

and pharmaceutically acceptable salts thereof,

(3) the therapeutic agent for type 2 diabetes according to (1) or (2), wherein the pharmaceutically acceptable salt is a salt with an acid selected from hydrochloric acid, nitric acid, sulfuric acid, sulfonic acid having 1 to 10 carbon atoms, a substituted or unsubstituted alkylcarboxylic acid having 1 to 6 carbon atoms, and a substituted or unsubstituted dicarboxylic acid having 4 to 8 carbon atoms,

(4) the therapeutic agent for type 2 diabetes according to (3), wherein the pharmaceutically acceptable salt is a salt with an acid selected from hydrochloric acid, nitric acid, methanesulfonic acid, acetic acid, levulinic acid, lactic acid, flurbiprofen, ketoprofen, fumaric acid, and maleic acid,

(5) the therapeutic agent for type 2 diabetes according to any one of (1) to (4), wherein the type 2 diabetes is type 2 diabetes with a reduced ability to secrete insulin caused by Cdkal1 gene mutation, and

(6) the therapeutic agent for type 2 diabetes according to any one of (1) to (5), wherein the therapeutic agent activates the conversion of proinsulin to insulin.

The present invention further relates to:

(7) a compound represented by the following formula (IV):

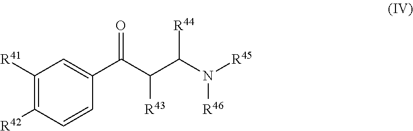

[wherein R⁴¹ represents any one group selected from hydrogen, halogen, a hydroxyl group, a substituted or unsubstituted linear or branched alkyl group having 1 to 4 carbon atoms, a substituted or unsubstituted linear or branched alkenyl group having 2 to 4 carbon atoms, a substituted or unsubstituted linear or branched alkynyl group having 2 to 4 carbon atoms, and a substituted or unsubstituted linear or branched alkoxy group having 1 to 4 carbon atoms,

R⁴² represents any one group selected from hydrogen, halogen, a hydroxyl group, a substituted or unsubstituted linear or branched alkyl group having 1 to 4 carbon atoms, a substituted or unsubstituted linear or branched alkenyl group having 2 to 4 carbon atoms, a substituted or unsubstituted linear or branched alkynyl group having 2 to 4 carbon atoms, and a substituted or unsubstituted linear or branched alkoxy group having 1 to 4 carbon atoms,

R⁴³ represents any one group selected from hydrogen, halogen, a hydroxyl group, a substituted or unsubstituted linear or branched alkyl group having 1 to 4 carbon atoms, a substituted or unsubstituted linear or branched alkenyl group having 2 to 4 carbon atoms, a substituted or unsubstituted linear or branched alkynyl group having 2 to 4 carbon atoms, and a substituted or unsubstituted linear or branched alkoxy group having 1 to 4 carbon atoms,

R⁴⁴ represents any one group selected from hydrogen, a carboxyl group, a substituted or unsubstituted linear or branched alkyl group having 1 to 4 carbon atoms, and a substituted or unsubstituted aryl group having 6 to 10 carbon atoms,

R⁴⁵ represents any one group selected from hydrogen, a substituted or unsubstituted linear or branched alkyl group having 1 to 4 carbon atoms, a substituted or unsubstituted linear or branched alkenyl group having 2 to 4 carbon atoms, and a substituted or unsubstituted linear or branched alkynyl group having 2 to 4 carbon atoms,

R⁴⁶ represents any one group selected from hydrogen, a substituted or unsubstituted linear or branched alkyl group having 1 to 4 carbon atoms, a substituted or unsubstituted linear or branched alkenyl group having 2 to 4 carbon atoms, a substituted or unsubstituted linear or branched alkynyl group having 2 to 4 carbon atoms, a substituted or unsubstituted aryl group having 5 to 10 carbon atoms, and an arylalkyl group consisting of a substituted or unsubstituted linear or branched alkyl group having 1 to 4 carbon atoms, and a substituted or unsubstituted aryl group having 5 to 10 carbon atoms, and

R⁴⁵ and R⁴⁶ optionally constitute a substituted or unsubstituted nitrogen-containing heterocyclic ring together with the nitrogen atom bonded thereto], and

(8) the compound according to (7), wherein the compound represented by the formula (IV) is represented by any of the following formulas (IV-1) to (IV-4):

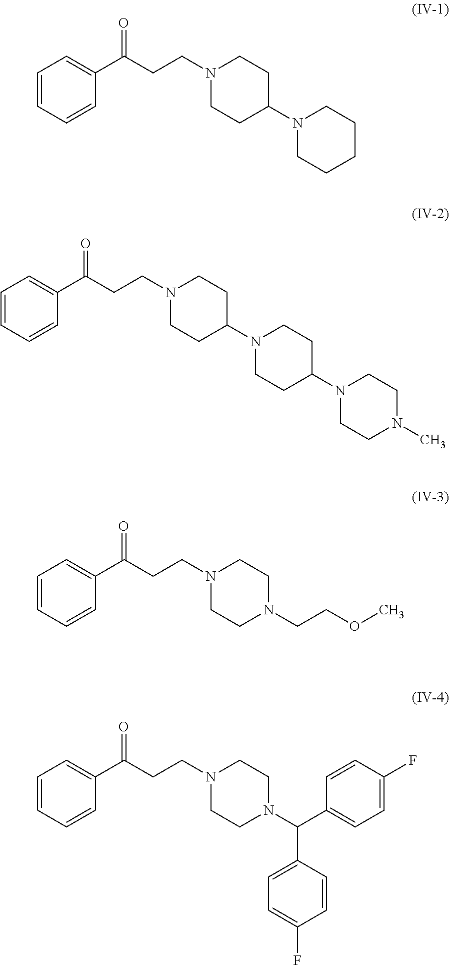 [SMILES: O=C(CCN1CCC(N2CCCCC2)CC1)c1ccccc1 ; CN1CCN(C2CCN(C3CCN(CCC(=O)c4ccccc4)CC3)CC2)CC1 ; COCCN1CCN(CCC(=O)c2ccccc2)CC1 ; O=C(CCN1CCN(C(c2ccc(F)cc2)c2ccc(F)cc2)CC1)c1ccccc1]

According to other aspects, the present invention relates to a method for treating type 2 diabetes, comprising administering a therapeutically effective amount of a compound represented by any of the formulas (I) to (III) or a pharmacologically acceptable salt thereof to a subject, use of a compound represented by any of the formulas (I) to (III) or a pharmacologically acceptable salt thereof for producing a therapeutic agent for type 2 diabetes, and a compound represented by any of the formulas (I) to (III) or a pharmacologically acceptable salt thereof for use in the treatment of type 2 diabetes.

### Effect of the Invention

The present invention provides a novel therapeutic agent for a patient with type 2 diabetes with Cdkal1 gene mutation resulting in the reduced ability to secrete insulin. Conventional therapeutic agents, which accelerate only insulin secretion in a patient with type 2 diabetes having Cdkal1 gene mutation, without improving translation accuracy, might cause the exhaustion of pancreatic β cells, leading to the aggravation of pathological conditions of type 2 diabetes. In contrast to this, the compounds represented by the formulas (I) to (III) or pharmaceutically acceptable salts thereof provided by the present invention can improve mistranslation caused by Cdkal1 gene mutation in pancreatic β cells and consequently achieve curative treatment by increasing the ability to secrete insulin.

### BRIEF DESCRIPTION OF DRAWINGS

FIG. 1 is a diagram summarizing the screening of a low-molecular compound that improves the accuracy of translation (primary screening).

FIG. 2 is a diagram showing results of studying the influence of the compound primarily screened for on translation accuracy.

FIG. 3 is a diagram showing results of conducting primary screening on existing therapeutic agents for type 2 diabetes.

FIG. 4 is a diagram showing results of conducting primary screening on eperisone.

FIG. 5 is a diagram showing results of conducting primary screening on fluoxetine.

FIG. 6 is a diagram showing results of conducting primary screening on elvitegravir.

FIG. 7 is a diagram showing results of conducting primary screening on compounds represented by the formulas I-2, I-3, I-4, I-5, and I-6, which are a compound group that was modified on the basis of a skeleton common to eperisone and fluoxetine.

FIG. 8 is a diagram showing results of relatively comprising the amount of insulin secreted by administering glibenclamide (therapeutic agent for type 2 diabetes classified as a sulfonylurea drug) or eperisone found positive in the primary screening to the pancreatic islet of Langerhans isolated from a pancreatic β cell-specific Cdkal1-deficient mouse (secondary screening).

FIG. 9 is a diagram showing results of conducting secondary screening on fluoxetine.

FIG. 10 is a diagram showing results of studying glucose tolerance by administering eperisone to a pancreatic β cell-specific Cdkal1-deficient mouse, then injecting a glucose solution thereto, and measuring the blood glucose level and the serum insulin concentration over time (tertiary screening).

FIG. 11 is a diagram showing results of studying glucose tolerance by administering eperisone to a pancreatic β cell-specific Cdkal1-deficient mouse in a long period (14 days), then injecting a glucose solution thereto, and measuring the blood glucose level and the serum insulin concentration over time.

FIG. 12 is a diagram showing results of studying glucose tolerance by administering fluoxetine to a pancreatic β cell-specific Cdkal1-deficient mouse in a long period (14 days), then injecting a glucose solution thereto, and measuring the blood glucose level and the serum insulin concentration over time.

### MODE OF CARRYING OUT THE INVENTION

R¹ in the formula (I) is any one group selected from hydrogen, a substituted or unsubstituted linear or branched alkyl group having 1 to 4 carbon atoms, a substituted or unsubstituted linear or branched alkenyl group having 2 to 4 carbon atoms, and a substituted or unsubstituted linear or branched alkynyl group having 2 to 4 carbon atoms.

Specific examples of the linear or branched alkyl group having 1 to 4 carbon atoms represented by R¹ can include a methyl group, an ethyl group, a propyl group, an isopropyl group, a n-butyl group, an isobutyl group, a sec-butyl group, and a tert-butyl group.

Specific examples of the linear or branched alkenyl group having 2 to 4 carbon atoms represented by R¹ can include a vinyl group, a 1-propenyl group, an allyl group, an isopropenyl group, a 1-butenyl group, a 2-butenyl group, a 3-butenyl group, a 1,3-dibutenyl group, a 1-ethylvinyl group, a 1-methyl-1-propenyl group, a 2-methyl-1-propenyl group, and a 2-methyl-2-propenyl group.

Specific examples of the linear or branched alkynyl group having 2 to 4 carbon atoms represented by R¹ can include an ethynyl group, a 1-propynyl group, a 2-propynyl group, a 1-butynyl group, a 2-butynyl group, a 3-butynyl group, a 1,3-dibutynyl group, and a 1-methyl-2-propynyl group.

The linear or branched alkyl group having 1 to 4 carbon atoms, the linear or branched alkenyl group having 2 to 4 carbon atoms, and the linear or branched alkynyl group having 2 to 4 carbon atoms may each be substituted, and examples of the substituent can include halogen (e.g., fluorine, chlorine, bromine, and iodine) and a hydroxyl group.

R¹ mentioned above can be any of the groups listed above and is preferably hydrogen, a methyl group, an ethyl group, a vinyl group, or an ethynyl group, more preferably hydrogen, a methyl group, or an ethynyl group, most preferably hydrogen.

R² in the formula (I) is any one group selected from hydrogen, halogen, a hydroxyl group, a substituted or unsubstituted linear or branched alkyl group having 1 to 10 carbon atoms, a substituted or unsubstituted linear or branched alkenyl group having 2 to 10 carbon atoms, a substituted or unsubstituted linear or branched alkynyl group having 2 to 10 carbon atoms, a substituted or unsubstituted alicyclic group having 3 to 10 carbon atoms, a substituted or unsubstituted aryl group having 6 to 10 carbon atoms, a substituted or unsubstituted alkoxy group having 1 to 10 carbon atoms, a substituted or unsubstituted acyl group having 1 to 11 carbon atoms, a carboxyl group and an ester derivative or an amide derivative thereof, a substituted or unsubstituted sulfonyl group having 1 to 10 carbon atoms, and a substituted or unsubstituted sulfide group having 1 to 10 carbon atoms.

Specific examples of the linear or branched alkyl group having 1 to 10 carbon atoms represented by R² can include a methyl group, an ethyl group, a propyl group, an isopropyl group, a n-butyl group, an isobutyl group, a sec-butyl group, a tert-butyl group, a n-pentyl group, a 1-methylbutyl group, a 2-methylbutyl group, a 3-methylbutyl group, a 1,1-dimethylpropyl group, a 1,2-dimethylpropyl group, a 2,2-dimethylpropyl group, a 3-pentyl group, a n-hexyl group, a 1-methylheptyl group, a 2-methylheptyl group, a 3-methylheptyl group, a 4-methylheptyl group, a 1,1-dimethylbutyl group, a 1,2-dimethylbutyl group, a 1,3-dimethylbutyl group, a 2,2-dimethylbutyl group, a 2,3-dimethylbutyl group, a 3,3-dimethylbutyl group, a 3,3-dimethylbutan-2-yl group, a 2,3-dimethylbutan-2-yl group, a 3-hexyl group, a 2-ethylpentyl group, a 2-methylpentan-3-yl group, a heptyl group, an octyl group, a nonyl group, and a decyl group.

Specific examples of the linear or branched alkenyl group having 2 to 10 carbon atoms represented by R² can include a vinyl group, a 1-propenyl group, an allyl group, an isopropenyl group, a 1-butenyl group, a 2-butenyl group, a 3-butenyl group, a 1,3-butanedienyl group, a 1-ethylvinyl group, a 1-methyl-1-propenyl group, a 2-methyl-1-propenyl group, a 2-methyl-2-propenyl group, a pentenyl group, a pentadienyl group, a hexenyl group, a hexadienyl group, a hexatrienyl group, a heptenyl group, a heptadienyl group, a heptatrienyl group, an octenyl group, an octadienyl group, an octatrienyl group, an octatetraenyl group, a nonenyl group, a nonadienyl group, a nonatrienyl group, a nonatetraenyl group, a decenyl group, a decadienyl group, a decatrienyl group, a decatetraenyl group, and a decapentaenyl group.

Specific examples of the linear or branched alkynyl group having 2 to 10 carbon atoms represented by R² can include an ethynyl group, a 1-propynyl group, a 2-propynyl group, a 1-butynyl group, a 2-butynyl group, a 3-butynyl group, a 1,3-butanediynyl group, a 1-methyl-2-propynyl group, a pentynyl group, a pentadiynyl group, a hexynyl group, a hexadiynyl group, a hexatriynyl group, a heptynyl group, a heptadiynyl group, a heptatriynyl group, an octynyl group, an octadiynyl group, an octatriynyl group, an octatetraynyl group, a nonynyl group, a nonadiynyl group, a nonatriynyl group, a nonatetraynyl group, a decynyl group, a decadiynyl group, a decatriynyl group, a decatetraynyl group, and a decapentaynyl group.

The linear or branched alkyl group having 1 to 10 carbon atoms, the linear or branched alkenyl group having 2 to 10 carbon atoms, and the linear or branched alkynyl group having 2 to 10 carbon atoms may each be substituted, and examples of the substituent can include halogen (e.g., fluorine, chlorine, bromine, and iodine), a hydroxyl group, a carboxyl group or an ester derivative thereof, a cyano group, and an alicyclic group mentioned later.

Specific examples of the alicyclic group having 3 to 10 carbon atoms represented by R² can include a cyclopropyl group, a cyclopropenyl group, a cyclobutyl group, a cyclobutenyl group, a cyclobutadienyl group, a cyclopentyl group, a cyclopentenyl group, a cyclopentadienyl group, a cyclohexyl group, a cyclohexenyl group, a cyclohexadienyl group, a cycloheptyl group, a cycloheptenyl group, a cycloheptadienyl group, a cycloheptatrienyl group, a bicycloheptyl group, a cyclooctyl group, a cyclooctenyl group, a cyclooctadienyl group, a cyclooctatrienyl group, a bicyclooctyl group, a cyclononyl group, a cyclononenyl group, a cyclononadienyl group, a bicyclononyl group, a cyclodecyl group, a cyclodecenyl group, a cyclodecadienyl group, a bicyclodecyl group, and an adamantyl group.

The alicyclic group having 3 to 10 carbon atoms may be substituted, and examples of the substituent can include halogen (e.g., fluorine, chlorine, bromine, and iodine), a hydroxyl group, the aforementioned linear or branched alkyl group having 1 to 4 carbon atoms, linear or branched alkenyl group having 2 to 4 carbon atoms, and linear or branched alkynyl group having 2 to 4 carbon atoms. The alicyclic group having 3 to 10 carbon atoms may be ring-fused with an aryl group, a nitrogen-containing aromatic group, or a heterocyclic group.

Specific examples of the aryl group having 6 to 10 carbon atoms represented by R² can include a phenyl group and a naphthyl group. The aryl group may be substituted, and examples of the substituent can include halogen (e.g., fluorine, chlorine, bromine, and iodine), a hydroxyl group, the aforementioned linear or branched alkyl group having 1 to 4 carbon atoms, linear or branched alkenyl group having 2 to 4 carbon atoms, and linear or branched alkynyl group having 2 to 4 carbon atoms. The aryl group having 6 to 10 carbon atoms may be ring-fused with an alicyclic group, a nitrogen-containing aromatic group, or a heterocyclic group.

Examples of the alkoxy group having 1 to 10 carbon atoms represented by R² can include a group bonded via an oxygen atom to the aforementioned linear or branched alkyl group having 1 to 10 carbon atoms, linear or branched alkenyl group having 2 to 10 carbon atoms, linear or branched alkynyl group having 2 to 10 carbon atoms, alicyclic group having 3 to 10 carbon atoms, aryl group having 6 to 10 carbon atoms, or the like. The alkoxy group having 1 to 10 carbon atoms may be substituted by a substituent such as halogen (e.g., fluorine, chlorine, bromine, and iodine) or a hydroxyl group.

Examples of the acyl group having 1 to 11 carbon atoms represented by R² can include a group bonded via carbonyl to hydrogen, the aforementioned linear or branched alkyl group having 1 to 10 carbon atoms, linear or branched alkenyl group having 2 to 10 carbon atoms, linear or branched alkynyl group having 2 to 10 carbon atoms, alicyclic group having 3 to 10 carbon atoms, aryl group having 6 to 10 carbon atoms, or the like. The acyl group having 1 to 11 carbon atoms may be substituted by a substituent such as halogen (e.g., fluorine, chlorine, bromine, and iodine) or a hydroxyl group.

Examples of the ester derivative of the carboxyl group represented by R² can include a carboxyl group in which hydrogen is substituted by the aforementioned linear or branched alkyl group having 1 to 10 carbon atoms, linear or branched alkenyl group having 2 to 10 carbon atoms, linear or branched alkynyl group having 2 to 10 carbon atoms, alicyclic group having 3 to 10 carbon atoms, aryl group having 6 to 10 carbon atoms, or the like. The ester derivative may be substituted, and examples of the substituent can include halogen (e.g., fluorine, chlorine, bromine, and iodine) and a hydroxyl group.

Examples of the amide derivative of the carboxyl group represented by R² can include a carboxyl group in which the hydroxyl group is substituted by nitrogen bonded to 1 or 2 aforementioned linear or branched alkyl groups having 1 to 10 carbon atoms, linear or branched alkenyl groups having 2 to 10 carbon atoms, linear or branched alkynyl groups having 2 to 10 carbon atoms, alicyclic groups having 3 to 10 carbon atoms, or aryl groups having 6 to 10 carbon atoms. The amide derivative may be substituted, and examples of the substituent can include halogen (e.g., fluorine, chlorine, bromine, and iodine) and a hydroxyl group.

Examples of the sulfonyl group having 1 to 10 carbon atoms represented by R² can include a group bonded via a sulfonyl group (—SO₂—) to the aforementioned linear or branched alkyl group having 1 to 10 carbon atoms, linear or branched alkenyl group having 2 to 10 carbon atoms, linear or branched alkynyl group having 2 to 10 carbon atoms, alicyclic group having 3 to 10 carbon atoms, aryl group having 6 to 10 carbon atoms, or the like. The sulfonyl group having 1 to 10 carbon atoms may be substituted by a substituent such as halogen (e.g., fluorine, chlorine, bromine, and iodine) or a hydroxyl group.

Examples of the sulfide group having 1 to 10 carbon atoms represented by R² can include a group bonded via a sulfur atom to the aforementioned linear or branched alkyl group having 1 to 10 carbon atoms, linear or branched alkenyl group having 2 to 10 carbon atoms, linear or branched alkynyl group having 2 to 10 carbon atoms, alicyclic group having 3 to 10 carbon atoms, aryl group having 6 to 10 carbon atoms, or the like. The sulfide group having 1 to 10 carbon atoms may be substituted by a substituent such as halogen (e.g., fluorine, chlorine, bromine, and iodine) or a hydroxyl group.

R² mentioned above can be any of the groups listed above and is preferably hydrogen, fluorine, chlorine, bromine, a hydroxyl group, an ethyl group, a vinyl group, an ethynyl group, a n-propyl group, an isopropyl group, a n-butyl group, an isobutyl group, a sec-butyl group, a tert-butyl group, a n-pentyl group, a n-hexyl group, a n-heptyl group, a n-octyl group, a n-nonyl group, a n-decyl group, a 1-hydroxyethyl group, a 2-hydroxyethyl group, a 1,2-dihydroxyethyl group, a 1-methoxycarbonylethyl group, a 1-ethoxycarbonylethyl group, a cyclopropylmethyl group, a cyclobutylmethyl group, a cyclopentylmethyl group, a cyclohexylmethyl group, a cyclopropyl group, a cyclobutyl group, a cyclopentyl group, a cyclohexyl group, a phenyl group, a tolyl group, a hydroxyphenyl group, a naphthyl group, a methylnaphthyl group, a hydroxynaphthyl group, an indanyl group, a tetrahydronaphthyl group, a methoxy group, an ethoxy group, a n-propoxy group, an isopropoxy group, a n-butoxy group, a tert-butoxy group, a formyl group, an acetyl group, a propionyl group, an acryloyl group, a propioloyl group, a methacryloyl group, a crotonyl group, an isocrotonyl group, a methylamino group, a dimethylamino group, an ethylamino group, a diethylamino group, a propylamino group, a dipropylamino group, a piperidino group, a piperazino group, a morpholino group, a methanesulfonyl group, an ethanesulfonyl group, a n-propanesulfonyl group, an isopropanesulfonyl group, a methyl sulfide group, an ethyl sulfide group, a n-propyl sulfide group, an isopropyl sulfide group, or a n-butyl sulfide group, more preferably hydrogen, fluorine, chlorine, a hydroxyl group, a methyl group, an ethyl group, a n-propyl group, an isopropyl group, a n-butyl group, an isobutyl group, a sec-butyl group, a tert-butyl group, a n-hexyl group, a n-heptyl group, a n-octyl group, a n-decyl group, a 1-hydroxyethyl group, a 2-hydroxyethyl group, a 1,2-dihydroxyethyl group, a 1-ethoxycarbonylethyl group, a cyclopropylmethyl group, a cyclohexylmethyl group, a cyclopentyl group, a cyclohexyl group, a phenyl group, a hydroxynaphthyl group, an indanyl group, a tetrahydronaphthyl group, a methoxy group, an ethoxy group, a n-propoxy group, an isopropoxy group, a n-butoxy group, a tert-butoxy group, an acetyl group, a dimethylamino group, a diethylamino group, a methanesulfonyl group, an ethanesulfonyl group, a methyl sulfide group, an ethyl sulfide group, or a n-propyl sulfide group, further preferably hydrogen, fluorine, chlorine, a hydroxyl group, a methyl group, an ethyl group, a n-propyl group, an isopropyl group, a n-butyl group, a tert-butyl group, a methoxy group, an ethoxy group, a n-propoxy group, an isopropoxy group, a methanesulfonyl group, or a n-propyl sulfide group.

R³ in the formula (I) is any one group selected from hydrogen, halogen, a substituted or unsubstituted linear or branched alkyl group having 1 to 4 carbon atoms, a substituted or unsubstituted linear or branched alkenyl group having 2 to 4 carbon atoms, a substituted or unsubstituted linear or branched alkynyl group having 2 to 4 carbon atoms, a substituted or unsubstituted aryl group having 6 to 10 carbon atoms, and a substituted or unsubstituted linear or branched alkoxy group having 1 to 4 carbon atoms.

Specific examples of the linear or branched alkyl group having 1 to 4 carbon atoms represented by R³ can include a methyl group, an ethyl group, a propyl group, an isopropyl group, a n-butyl group, an isobutyl group, a sec-butyl group, and a tert-butyl group.

Specific examples of the linear or branched alkenyl group having 2 to 4 carbon atoms represented by R³ can include a vinyl group, a 1-propenyl group, an allyl group, an isopropenyl group, a 1-butenyl group, a 2-butenyl group, a 3-butenyl group, a 1,3-dibutenyl group, a 1-ethylvinyl group, a 1-methyl-1-propenyl group, a 2-methyl-1-propenyl group, and a 2-methyl-2-propenyl group.

Specific examples of the linear or branched alkynyl group having 2 to 4 carbon atoms represented by R³ can include an ethynyl group, a 1-propynyl group, a 2-propynyl group, a 1-butynyl group, a 2-butynyl group, a 3-butynyl group, a 1,3-dibutynyl group, and a 1-methyl-2-propynyl group.

The linear or branched alkyl group having 1 to 4 carbon atoms, the linear or branched alkenyl group having 2 to 4 carbon atoms, and the linear or branched alkynyl group having 2 to 4 carbon atoms may each be substituted, and examples of the substituent can include halogen (e.g., fluorine, chlorine, bromine, and iodine) and a hydroxyl group.

Specific examples of the aryl group having 6 to 10 carbon atoms represented by R³ can include a phenyl group and a naphthyl group. The aryl group may be substituted, and examples of the substituent can include halogen (e.g., fluorine, chlorine, bromine, and iodine), a hydroxyl group, the aforementioned linear or branched alkyl group having 1 to 4 carbon atoms, linear or branched alkenyl group having 2 to 4 carbon atoms, and linear or branched alkynyl group having 2 to 4 carbon atoms. The aryl group having 6 to 10 carbon atoms may be ring-fused with an alicyclic group, a nitrogen-containing aromatic group, or a heterocyclic group.

Examples of the linear or branched alkoxy group having 1 to 4 carbon atoms represented by R³ can include a group bonded via an oxygen atom to the aforementioned linear or branched alkyl group having 1 to 4 carbon atoms, linear or branched alkenyl group having 2 to 4 carbon atoms, linear or branched alkynyl group having 2 to 4 carbon atoms, or the like. The alkoxy group may be substituted by a substituent such as halogen (e.g., fluorine, chlorine, bromine, and iodine) or a hydroxyl group.

R³ mentioned above can be any of the groups listed above and is preferably hydrogen, fluorine, chlorine, a methyl group, an ethyl group, a vinyl group, an ethynyl group, a phenyl group, a naphthyl group, a methoxy group, or an ethoxy group, more preferably hydrogen, fluorine, an ethyl group, a phenyl group, or a methoxy group, further preferably hydrogen, fluorine, or a methoxy group.

R⁴ in the formula (I) is any one group selected from hydrogen, a substituted or unsubstituted aryl group having 6 to 10 carbon atoms, and a substituted or unsubstituted aryloxy group having 6 to 10 carbon atoms, or is a group that forms a carbonyl group together with R⁵ and carbon bonded thereto.

Specific examples of the aryl group having 6 to 10 carbon atoms represented by R⁴ can include a phenyl group and a naphthyl group. The aryl group may be substituted, and examples of the substituent can include halogen (e.g., fluorine, chlorine, bromine, and iodine), a hydroxyl group, and a linear or branched alkyl group having 1 to 4 carbon atoms optionally substituted by halogen, a hydroxyl group, an amino group, a carboxyl group, or the like. The aryl group having 6 to 10 carbon atoms may be ring-fused with an alicyclic group, a nitrogen-containing aromatic group, or a heterocyclic group.

Examples of the aryloxy group having 6 to 10 carbon atoms represented by R⁴ can include a group bonded via an oxygen atom to the aforementioned substituted or unsubstituted aryl group having 6 to 10 carbon atoms.

R⁴ mentioned above can be any of the groups listed above and is preferably hydrogen, a phenyl group, a methylphenyl group, a trifluoromethylphenyl group, a phenoxy group, a methylphenoxy group, or a trifluoromethylphenoxy group, more preferably hydrogen, a methylphenoxy group, or a trifluoromethylphenoxy group, further preferably hydrogen or a trifluoromethylphenoxy group.

R⁴ mentioned above optionally forms a carbonyl group together with R⁵ and carbon bonded thereto.

R⁵ in the formula (I) is hydrogen, or is a group that forms a carbonyl group together with R⁴ and carbon bonded thereto.

R⁶ in the formula (I) is any one group selected from hydrogen, halogen, a substituted or unsubstituted linear or branched alkyl group having 1 to 6 carbon atoms, a substituted or unsubstituted linear or branched alkenyl group having 2 to 6 carbon atoms, a substituted or unsubstituted linear or branched alkynyl group having 2 to 6 carbon atoms, a substituted or unsubstituted alicyclic group having 3 to 6 carbon atoms, a substituted or unsubstituted linear or branched alkyloxycarbonyl group having 1 to 4 carbon atoms, a substituted or unsubstituted linear or branched alkenyloxycarbonyl group having 2 to 4 carbon atoms, a substituted or unsubstituted linear or branched alkynyloxycarbonyl group having 2 to 4 carbon atoms, a substituted or unsubstituted aryl group having 6 to 10 carbon atoms, a carboxyl group and an ester derivative or an amide derivative thereof, a cyano group, and an amino group.

Specific examples of the linear or branched alkyl group having 1 to 6 carbon atoms represented by R⁶ can include a methyl group, an ethyl group, a propyl group, an isopropyl group, a n-butyl group, an isobutyl group, a sec-butyl group, a tert-butyl group, a n-pentyl group, a 1-methylbutyl group, a 2-methylbutyl group, a 3-methylbutyl group, a 1,1-dimethylpropyl group, a 1,2-dimethylpropyl group, a 2,2-dimethylpropyl group, a 3-pentyl group, a n-hexyl group, a 1-methylheptyl group, a 2-methylheptyl group, a 3-methylheptyl group, a 4-methylheptyl group, a 1,1-dimethylbutyl group, a 1,2-dimethylbutyl group, a 1,3-dimethylbutyl group, a 2,2-dimethylbutyl group, a 2,3-dimethylbutyl group, a 3,3-dimethylbutyl group, a 3,3-dimethylbutan-2-yl group, a 2,3-dimethylbutan-2-yl group, a 3-hexyl group, a 2-ethylpentyl group, and a 2-methylpentan-3-yl group.

Specific examples of the linear or branched alkenyl group having 2 to 6 carbon atoms represented by R⁶ can include a vinyl group, a 1-propenyl group, an allyl group, an isopropenyl group, a 1-butenyl group, a 2-butenyl group, a 3-butenyl group, a 1,3-butanedienyl group, a 1-ethylvinyl group, a 1-methyl-1-propenyl group, a 2-methyl-1-propenyl group, a 2-methyl-2-propenyl group, a pentenyl group, a pentadienyl group, a hexenyl group, a hexadienyl group, and a hexatrienyl group.

Specific examples of the linear or branched alkynyl group having 2 to 6 carbon atoms represented by R⁶ can include an ethynyl group, a 1-propynyl group, a 2-propynyl group, a 1-butynyl group, a 2-butynyl group, a 3-butynyl group, a 1,3-butanediynyl group, a 1-methyl-2-propynyl group, a pentynyl group, a pentadiynyl group, a hexynyl group, a hexadiynyl group, and a hexatriynyl group.

The linear or branched alkyl group having 1 to 6 carbon atoms, the linear or branched alkenyl group having 2 to 6 carbon atoms, and the linear or branched alkynyl group having 2 to 6 carbon atoms may each be substituted, and examples of the substituent can include halogen (e.g., fluorine, chlorine, bromine, and iodine), a hydroxyl group, a carboxyl group or an ester derivative thereof, a cyano group, an alicyclic group, and a nitrogen-containing heterocyclic group.

Specific examples of the alicyclic group having 3 to 6 carbon atoms represented by R⁶ can include a cyclopropyl group, a cyclopropenyl group, a cyclobutyl group, a cyclobutenyl group, a cyclobutadienyl group, a cyclopentyl group, a cyclopentenyl group, a cyclopentadienyl group, a cyclohexyl group, a cyclohexenyl group, and a cyclohexadienyl group. The alicyclic group may be substituted, and examples of the substituent can include halogen (e.g., fluorine, chlorine, bromine, and iodine), a hydroxyl group, the aforementioned linear or branched alkyl group having 1 to 4 carbon atoms, linear or branched alkenyl group having 2 to 4 carbon atoms, and linear or branched alkynyl group having 2 to 4 carbon atoms. The alicyclic group may be ring-fused with an aryl group, a nitrogen-containing aromatic group, or a heterocyclic group.

Specific examples of the linear or branched alkyloxycarbonyl group having 1 to 4 carbon atoms represented by R⁶ can include a methoxycarbonyl group, an ethoxycarbonyl group, a propyloxycarbonyl group, an isopropyloxycarbonyl group, a n-butoxycarbonyl group, an isobutoxycarbonyl group, a sec-butoxycarbonyl group, and a tert-butoxycarbonyl group.

Specific examples of the linear or branched alkenyloxycarbonyl group having 2 to 4 carbon atoms represented by R⁶ can include a vinyloxycarbonyl group, a 1-propenyloxycarbonyl group, an allyloxycarbonyl group, an isopropenyloxycarbonyl group, a 1-butenyloxycarbonyl group, a 2-butenyloxycarbonyl group, a 3-butenyloxycarbonyl group, a 1,3-dibutenyloxycarbonyl group, a 1-ethylvinyloxycarbonyl group, a 1-methyl-1-propenyloxycarbonyl group, a 2-methyl-1-propenyloxycarbonyl group, and a 2-methyl-2-propenyloxycarbonyl group.

Specific examples of the linear or branched alkynyloxycarbonyl group having 2 to 4 carbon atoms represented by R⁶ can include an ethynyloxycarbonyl group, a 1-propynyloxycarbonyl group, a 2-propynyloxycarbonyl group, a 1-butynyloxycarbonyl group, a 2-butynyloxycarbonyl group, a 3-butynyloxycarbonyl group, a 1,3-butanediynyloxycarbonyl group, and a 1-methyl-2-propynyloxycarbonyl group.

The linear or branched alkyloxycarbonyl group, the linear or branched alkenyloxycarbonyl group, and the linear or branched alkynyloxycarbonyl group may each be substituted, and examples of the substituent can include halogen (e.g., fluorine, chlorine, bromine, and iodine), a hydroxyl group, and a cyano group.

Specific examples of the aryl group having 6 to 10 carbon atoms represented by R⁶ can include a phenyl group and a naphthyl group. The aryl group may be substituted, and examples of the substituent can include halogen (e.g., fluorine, chlorine, bromine, and iodine), a hydroxyl group, the aforementioned linear or branched alkyl group having 1 to 4 carbon atoms, linear or branched alkenyl group having 2 to 4 carbon atoms, and linear or branched alkynyl group having 2 to 4 carbon atoms. The aryl group may be ring-fused with an alicyclic group, a nitrogen-containing aromatic group, or a heterocyclic group.

Examples of the ester derivative of the carboxyl group represented by R⁶ can include a carboxyl group in which hydrogen is substituted by an alicyclic group having 3 to 6 carbon atoms, an aryl group having 6 to 10 carbon atoms, or the like. The ester derivative may be substituted, and examples of the substituent can include halogen (e.g., fluorine, chlorine, bromine, and iodine) and a hydroxyl group.

Examples of the amide derivative of the carboxyl group represented by R⁶ can include a carboxyl group in which the hydroxyl group is substituted by nitrogen bonded to 1 or 2 aforementioned linear or branched alkyl groups having 1 to 6 carbon atoms, linear or branched alkenyl groups having 2 to 6 carbon atoms, linear or branched alkynyl groups having 2 to 6 carbon atoms, or alicyclic groups having 3 to 6 carbon atoms. The amide derivative may be substituted, and examples of the substituent can include halogen (e.g., fluorine, chlorine, bromine, and iodine) and a hydroxyl group.

Examples of the amino group represented by R⁶ can include an amino group, and an amino group in which 1 or 2 hydrogen atoms are substituted by 1 or 2 aforementioned linear or branched alkyl groups having 1 to 6 carbon atoms, linear or branched alkenyl groups having 2 to 6 carbon atoms, or linear or branched alkynyl groups having 2 to 6 carbon atoms. The substituted amino group may be further substituted, and examples of the substituent can include halogen (e.g., fluorine, chlorine, bromine, and iodine) and a hydroxyl group.

R⁶ mentioned above can be any of the groups listed above and is preferably hydrogen, fluorine, chlorine, bromine, iodine, a methyl group, an ethyl group, a vinyl group, an ethynyl group, a n-propyl group, an isopropyl group, a n-butyl group, an isobutyl group, a sec-butyl group, a tert-butyl group, a n-pentyl group, a n-hexyl group, a hydroxymethyl group, a carboxylmethyl group, a cyclopentylmethyl group, a cyclohexylmethyl group, a piperidinomethyl group, a methylpiperidinomethyl group, a methoxycarbonyl group, an ethoxycarbonyl group, a phenyl group, a tolyl group, a hydroxyphenyl group, a naphthyl group, a carboxyl group, a cyano group, an amino group, a dimethylamino group, or a diethylamino group, more preferably hydrogen, fluorine, a methyl group, an ethyl group, a n-propyl group, an isopropyl group, a n-butyl group, a n-pentyl group, a n-hexyl group, a hydroxymethyl group, a carboxylmethyl group, a cyclopentylmethyl group, a piperidinomethyl group, a methylpiperidinomethyl group, a methoxycarbonyl group, a cyano group, an amino group, or a dimethylamino group, further preferably hydrogen, fluorine, a methyl group, or an amino group.

R⁷ in the formula (I) is any one group selected from hydrogen, a substituted or unsubstituted linear or branched alkyl group having 1 to 4 carbon atoms, a substituted or unsubstituted linear or branched alkenyl group having 2 to 4 carbon atoms, and a substituted or unsubstituted linear or branched alkynyl group having 2 to 4 carbon atoms.

Specific examples of the linear or branched alkyl group having 1 to 4 carbon atoms represented by R⁷ can include a methyl group, an ethyl group, a propyl group, an isopropyl group, a n-butyl group, an isobutyl group, a sec-butyl group, and a tert-butyl group.

Specific examples of the linear or branched alkenyl group having 2 to 4 carbon atoms represented by R⁷ can include a vinyl group, a 1-propenyl group, an allyl group, an isopropenyl group, a 1-butenyl group, a 2-butenyl group, a 3-butenyl group, a 1,3-dibutenyl group, a 1-ethylvinyl group, a 1-methyl-1-propenyl group, a 2-methyl-1-propenyl group, and a 2-methyl-2-propenyl group.

Specific examples of the linear or branched alkynyl group having 2 to 4 carbon atoms represented by R⁷ can include an ethynyl group, a 1-propynyl group, a 2-propynyl group, a 1-butynyl group, a 2-butynyl group, a 3-butynyl group, a 1,3-dibutynyl group, and a 1-methyl-2-propynyl group.

The linear or branched alkyl group having 1 to 4 carbon atoms, the linear or branched alkenyl group having 2 to 4 carbon atoms, and the linear or branched alkynyl group having 2 to 4 carbon atoms may each be substituted, and examples of the substituent can include halogen (e.g., fluorine, chlorine, bromine, and iodine) and a hydroxyl group.

R⁷ mentioned above can be any of the groups listed above and is preferably hydrogen, a methyl group, an ethyl group, a vinyl group, or an ethynyl group, more preferably hydrogen, a methyl group, or an ethynyl group, most preferably hydrogen.

R⁸ in the formula (I) is any one group selected from hydrogen, halogen, a substituted or unsubstituted linear or branched alkyl group having 1 to 6 carbon atoms, a substituted or unsubstituted linear or branched alkenyl group having 2 to 6 carbon atoms, a substituted or unsubstituted linear or branched alkynyl group having 2 to 6 carbon atoms, a substituted or unsubstituted alicyclic group having 3 to 6 carbon atoms, a substituted or unsubstituted linear or branched alkyloxycarbonyl group having 1 to 4 carbon atoms, a substituted or unsubstituted linear or branched alkenyloxycarbonyl group having 2 to 4 carbon atoms, a substituted or unsubstituted linear or branched alkynyloxycarbonyl group having 2 to 4 carbon atoms, a substituted or unsubstituted aryl group having 6 to 10 carbon atoms, a carboxyl group and an ester derivative or an amide derivative thereof, a cyano group, and an amino group, or is a group that forms a carbonyl group together with R⁵ and carbon bonded thereto.

Specific examples of the linear or branched alkyl group having 1 to 6 carbon atoms represented by R⁸ can include a methyl group, an ethyl group, a propyl group, an isopropyl group, a n-butyl group, an isobutyl group, a sec-butyl group, a tert-butyl group, a n-pentyl group, a 1-methylbutyl group, a 2-methylbutyl group, a 3-methylbutyl group, a 1,1-dimethylpropyl group, a 1,2-dimethylpropyl group, a 2,2-dimethylpropyl group, a 3-pentyl group, a n-hexyl group, a 1-methylheptyl group, a 2-methylheptyl group, a 3-methylheptyl group, a 4-methylheptyl group, a 1,1-dimethylbutyl group, a 1,2-dimethylbutyl group, a 1,3-dimethylbutyl group, a 2,2-dimethylbutyl group, a 2,3-dimethylbutyl group, a 3,3-dimethylbutyl group, a 3,3-dimethylbutan-2-yl group, a 2,3-dimethylbutan-2-yl group, a 3-hexyl group, a 2-ethylpentyl group, and a 2-methylpentan-3-yl group.

Specific examples of the linear or branched alkenyl group having 2 to 6 carbon atoms represented by R⁸ can include a vinyl group, a 1-propenyl group, an allyl group, an isopropenyl group, a 1-butenyl group, a 2-butenyl group, a 3-butenyl group, a 1,3-butanedienyl group, a 1-ethylvinyl group, a 1-methyl-1-propenyl group, a 2-methyl-1-propenyl group, a 2-methyl-2-propenyl group, a pentenyl group, a pentadienyl group, a hexenyl group, a hexadienyl group, and a hexatrienyl group.

Specific examples of the linear or branched alkynyl group having 2 to 6 carbon atoms represented by R⁸ can include an ethynyl group, a 1-propynyl group, a 2-propynyl group, a 1-butynyl group, a 2-butynyl group, a 3-butynyl group, a 1,3-butanediynyl group, a 1-methyl-2-propynyl group, a pentynyl group, a pentadiynyl group, a hexynyl group, a hexadiynyl group, and a hexatriynyl group.

The linear or branched alkyl group having 1 to 6 carbon atoms, the linear or branched alkenyl group having 2 to 6 carbon atoms, and the linear or branched alkynyl group having 2 to 6 carbon atoms may each be substituted, and examples of the substituent can include halogen (e.g., fluorine, chlorine, bromine, and iodine), a hydroxyl group, a carboxyl group or an ester derivative thereof, a cyano group, an alicyclic group, and a nitrogen-containing heterocyclic group.

Specific examples of the alicyclic group having 3 to 6 carbon atoms represented by R⁸ can include a cyclopropyl group, a cyclopropenyl group, a cyclobutyl group, a cyclobutenyl group, a cyclobutadienyl group, a cyclopentyl group, a cyclopentenyl group, a cyclopentadienyl group, a cyclohexyl group, a cyclohexenyl group, and a cyclohexadienyl group. The alicyclic group may be substituted, and examples of the substituent can include halogen (e.g., fluorine, chlorine, bromine, and iodine), a hydroxyl group, the aforementioned linear or branched alkyl group having 1 to 4 carbon atoms, linear or branched alkenyl group having 2 to 4 carbon atoms, and linear or branched alkynyl group having 2 to 4 carbon atoms. The alicyclic group may be ring-fused with an aryl group, a nitrogen-containing aromatic group, or a heterocyclic group.

Specific examples of the linear or branched alkyloxycarbonyl group having 1 to 4 carbon atoms represented by R⁸ can include a methoxycarbonyl group, an ethoxycarbonyl group, a propyloxycarbonyl group, an isopropyloxycarbonyl group, a n-butoxycarbonyl group, an isobutoxycarbonyl group, a sec-butoxycarbonyl group, and a tert-butoxycarbonyl group.

Specific examples of the linear or branched alkenyloxycarbonyl group having 2 to 4 carbon atoms represented by R⁸ can include a vinyloxycarbonyl group, a 1-propenyloxycarbonyl group, an allyloxycarbonyl group, an isopropenyloxycarbonyl group, a 1-butenyloxycarbonyl group, a 2-butenyloxycarbonyl group, a 3-butenyloxycarbonyl group, a 1,3-dibutenyloxycarbonyl group, a 1-ethylvinyloxycarbonyl group, a 1-methyl-1-propenyloxycarbonyl group, a 2-methyl-1-propenyloxycarbonyl group, and a 2-methyl-2-propenyloxycarbonyl group.

Specific examples of the linear or branched alkynyloxycarbonyl group having 2 to 4 carbon atoms represented by R⁸ can include an ethynyloxycarbonyl group, a 1-propynyloxycarbonyl group, a 2-propynyloxycarbonyl group, a 1-butynyloxycarbonyl group, a 2-butynyloxycarbonyl group, a 3-butynyloxycarbonyl group, a 1,3-butanediynyloxycarbonyl group, and a 1-methyl-2-propynyloxycarbonyl group.

The linear or branched alkyloxycarbonyl group, the linear or branched alkenyloxycarbonyl group, and the linear or branched alkynyloxycarbonyl group may each be substituted, and examples of the substituent can include halogen (e.g., fluorine, chlorine, bromine, and iodine), a hydroxyl group, and a cyano group.

Specific examples of the aryl group having 6 to 10 carbon atoms represented by R⁸ can include a phenyl group and a naphthyl group. The aryl group may be substituted, and examples of the substituent can include halogen (e.g., fluorine, chlorine, bromine, and iodine), a hydroxyl group, the aforementioned linear or branched alkyl group having 1 to 4 carbon atoms, linear or branched alkenyl group having 2 to 4 carbon atoms, linear or branched alkynyl group having 2 to 4 carbon atoms, an alkoxy group substituted via an oxygen atom by the aforementioned linear or branched alkyl group having 1 to 4 carbon atoms, and a sulfide group substituted via a sulfur atom by the aforementioned linear or branched alkyl group having 1 to 4 carbon atoms. The aryl group may be ring-fused with an alicyclic group, a nitrogen-containing aromatic group, or a heterocyclic group.

Examples of the ester derivative of the carboxyl group represented by R⁸ can include a carboxyl group in which hydrogen is substituted by an alicyclic group having 3 to 6 carbon atoms, an aryl group having 6 to 10 carbon atoms, or the like. The ester derivative may be substituted, and examples of the substituent can include halogen (e.g., fluorine, chlorine, bromine, and iodine) and a hydroxyl group.

Examples of the amide derivative of the carboxyl group represented by R⁸ can include a carboxyl group in which the hydroxyl group is substituted by nitrogen bonded to 1 or 2 aforementioned linear or branched alkyl groups having 1 to 6 carbon atoms, linear or branched alkenyl groups having 2 to 6 carbon atoms, linear or branched alkynyl groups having 2 to 6 carbon atoms, alicyclic groups having 3 to 6 carbon atoms, aryl groups having 6 to 10 carbon atoms, or the like. The amide derivative may be substituted, and examples of the substituent can include halogen (e.g., fluorine, chlorine, bromine, and iodine) and a hydroxyl group.

Examples of the amino group represented by R⁸ can include an amino group, and an amino group in which 1 or 2 hydrogen atoms are substituted by 1 or 2 aforementioned linear or branched alkyl groups having 1 to 6 carbon atoms, linear or branched alkenyl groups having 2 to 6 carbon atoms, or linear or branched alkynyl groups having 2 to 6 carbon atoms. The substituted amino group may be further substituted, and examples of the substituent can include halogen (e.g., fluorine, chlorine, bromine, and iodine) and a hydroxyl group.

R⁸ mentioned above can be any of the groups listed above and is preferably hydrogen, fluorine, chlorine, bromine, iodine, a methyl group, an ethyl group, a vinyl group, an ethynyl group, a hydroxymethyl group, a carboxylmethyl group, a cyclopentylmethyl group, a cyclohexylmethyl group, a piperidinomethyl group, a methylpiperidinomethyl group, a methoxycarbonyl group, an ethoxycarbonyl group, a phenyl group, a tolyl group, an ethylphenyl group, a hydroxyphenyl group, a methoxyphenyl group, a dimethoxyphenyl group, an ethoxyphenyl group, a fluorophenyl group, a chlorophenyl group, a methylthiophenyl group, an ethylthiophenyl group, a naphthyl group, a carboxyl group, a cyano group, an amino group, a dimethylamino group, or a diethylamino group, more preferably hydrogen, fluorine, a methyl group, an ethyl group, a carboxylmethyl group, a methoxycarbonyl group, a phenyl group, a tolyl group, an ethylphenyl group, a hydroxyphenyl group, a methoxyphenyl group, a dimethoxyphenyl group, an ethoxyphenyl group, a fluorophenyl group, a chlorophenyl group, a methylthiophenyl group, an ethylthiophenyl group, a naphthyl group, a carboxyl group, a cyano group, an amino group, or a dimethylamino group, further preferably hydrogen, fluorine, a phenyl group, an ethylphenyl group, a methoxyphenyl group, a dimethoxyphenyl group, a chlorophenyl group, a methylthiophenyl group, or a carboxyl group.

R⁸ mentioned above optionally forms a carbonyl group together with R⁹ and carbon bonded thereto.

R⁹ in the formula (I) is hydrogen, or is a group that forms a carbonyl group together with R⁸ and carbon bonded thereto.

R¹⁰ in the formula (I) is any one group selected from hydrogen, a substituted or unsubstituted linear or branched alkyl group having 1 to 6 carbon atoms, a substituted or unsubstituted linear or branched alkenyl group having 2 to 6 carbon atoms, a substituted or unsubstituted linear or branched alkynyl group having 2 to 6 carbon atoms, a substituted or unsubstituted alicyclic group having 3 to 6 carbon atoms, a substituted or unsubstituted linear or branched alkyloxycarbonyl group having 1 to 6 carbon atoms, a substituted or unsubstituted linear or branched alkenyloxycarbonyl group having 2 to 6 carbon atoms, a substituted or unsubstituted linear or branched alkynyloxycarbonyl group having 2 to 6 carbon atoms, a substituted or unsubstituted aryl group having 6 to 10 carbon atoms, a carboxyl group and an ester derivative or an amide derivative thereof, a cyano group, and an amino group, or is a group that forms a substituted or unsubstituted nitrogen-containing heterocyclic ring together with R¹¹ and the nitrogen atom bonded thereto.

Specific examples of the linear or branched alkyl group having 1 to 6 carbon atoms represented by R¹⁰ can include a methyl group, an ethyl group, a propyl group, an isopropyl group, a n-butyl group, an isobutyl group, a sec-butyl group, a tert-butyl group, a n-pentyl group, a 1-methylbutyl group, a 2-methylbutyl group, a 3-methylbutyl group, a 1,1-dimethylpropyl group, a 1,2-dimethylpropyl group, a 2,2-dimethylpropyl group, a 3-pentyl group, a n-hexyl group, a 1-methylheptyl group, a 2-methylheptyl group, a 3-methylheptyl group, a 4-methylheptyl group, a 1,1-dimethylbutyl group, a 1,2-dimethylbutyl group, a 1,3-dimethylbutyl group, a 2,2-dimethylbutyl group, a 2,3-dimethylbutyl group, a 3,3-dimethylbutyl group, a 3,3-dimethylbutan-2-yl group, a 2,3-dimethylbutan-2-yl group, a 3-hexyl group, a 2-ethylpentyl group, and a 2-methylpentan-3-yl group.

Specific examples of the linear or branched alkenyl group having 2 to 6 carbon atoms represented by R¹⁰ can include a vinyl group, a 1-propenyl group, an allyl group, an isopropenyl group, a 1-butenyl group, a 2-butenyl group, a 3-butenyl group, a 1,3-butanedienyl group, a 1-ethylvinyl group, a 1-methyl-1-propenyl group, a 2-methyl-1-propenyl group, a 2-methyl-2-propenyl group, a pentenyl group, a pentadienyl group, a hexenyl group, a hexadienyl group, and a hexatrienyl group.

Specific examples of the linear or branched alkynyl group having 2 to 6 carbon atoms represented by R¹⁰ can include an ethynyl group, a 1-propynyl group, a 2-propynyl group, a 1-butynyl group, a 2-butynyl group, a 3-butynyl group, a 1,3-butanediynyl group, a 1-methyl-2-propynyl group, a pentynyl group, a pentadiynyl group, a hexynyl group, a hexadiynyl group, and a hexatriynyl group.

The linear or branched alkyl group having 1 to 6 carbon atoms, the linear or branched alkenyl group having 2 to 6 carbon atoms, and the linear or branched alkynyl group having 2 to 6 carbon atoms may each be substituted, and examples of the substituent can include halogen (e.g., fluorine, chlorine, bromine, and iodine), a hydroxyl group, a substituted or unsubstituted aryl group having 5 to 10 carbon atoms mentioned later, a carboxyl group or an ester derivative thereof, a cyano group, an alicyclic group, and a nitrogen-containing heterocyclic group.

Specific examples of the alicyclic group having 3 to 6 carbon atoms represented by R¹⁰ can include a cyclopropyl group, a cyclopropenyl group, a cyclobutyl group, a cyclobutenyl group, a cyclobutadienyl group, a cyclopentyl group, a cyclopentenyl group, a cyclopentadienyl group, a cyclohexyl group, a cyclohexenyl group, and a cyclohexadienyl group. The alicyclic group may be substituted, and examples of the substituent can include halogen (e.g., fluorine, chlorine, bromine, and iodine), a hydroxyl group, the aforementioned linear or branched alkyl group having 1 to 4 carbon atoms, linear or branched alkenyl group having 2 to 4 carbon atoms, and linear or branched alkynyl group having 2 to 4 carbon atoms. The alicyclic group may be ring-fused with an aryl group, a nitrogen-containing aromatic group, or a heterocyclic group.

Specific examples of the linear or branched alkyloxycarbonyl group having 1 to 6 carbon atoms represented by R¹⁰ can include a methoxycarbonyl group, an ethoxycarbonyl group, a propyloxycarbonyl group, an isopropyloxycarbonyl group, a n-butoxycarbonyl group, an isobutoxycarbonyl group, a sec-butoxycarbonyl group, and a tert-butoxycarbonyl group.

Specific examples of the linear or branched alkenyloxycarbonyl group having 2 to 6 carbon atoms represented by R¹⁰ can include a vinyloxycarbonyl group, a 1-propenyloxycarbonyl group, an allyloxycarbonyl group, an isopropenyloxycarbonyl group, a 1-butenyloxycarbonyl group, a 2-butenyloxycarbonyl group, a 3-butenyloxycarbonyl group, a 1,3-dibutenyloxycarbonyl group, a 1-ethylvinyloxycarbonyl group, a 1-methyl-1-propenyloxycarbonyl group, a 2-methyl-1-propenyloxycarbonyl group, and a 2-methyl-2-propenyloxycarbonyl group.

Specific examples of the linear or branched alkynyloxycarbonyl group having 2 to 6 carbon atoms represented by R¹⁰ can include an ethynyloxycarbonyl group, a 1-propynyloxycarbonyl group, a 2-propynyloxycarbonyl group, a 1-butynyloxycarbonyl group, a 2-butynyloxycarbonyl group, a 3-butynyloxycarbonyl group, a 1,3-butanediynyloxycarbonyl group, and a 1-methyl-2-propynyloxycarbonyl group.

The linear or branched alkyloxycarbonyl group, the linear or branched alkenyloxycarbonyl group, and the linear or branched alkynyloxycarbonyl group each form an amide carbonate structure together with the nitrogen atom bonded thereto. The amide carbonate structure may be substituted, and examples of the substituent can include halogen (e.g., fluorine, chlorine, bromine, and iodine), a hydroxyl group, and a cyano group.

Specific examples of the aryl group having 5 to 10 carbon atoms represented by R¹⁰ can include a phenyl group, a pyridyl group, an indolyl group, a quinolyl group, an isoquinolyl group, and a naphthyl group. The aryl group may be substituted, and examples of the substituent can include halogen (e.g., fluorine, chlorine, bromine, and iodine), a hydroxyl group, the aforementioned linear or branched alkyl group having 1 to 6 carbon atoms, linear or branched alkenyl group having 2 to 6 carbon atoms, linear or branched alkynyl group having 2 to 6 carbon atoms, an alkoxy group substituted via an oxygen atom by the aforementioned linear or branched alkyl group having 1 to carbon atoms, the aforementioned linear or branched alkyloxycarbonyl group having 1 to 6 carbon atoms, and a sulfide group substituted via a sulfur atom by the aforementioned linear or branched alkyl group having 1 to 6 carbon atoms. The aryl group may be ring-fused with an alicyclic group or a heterocyclic group.

Examples of the ester derivative of the carboxyl group represented by R¹⁰ can include a carboxyl group substituted by an alicyclic group having 3 to 6 carbon atoms, an aryl group having 5 to 10 carbon atoms, or the like. The ester derivative forms an amide carbonate structure together with the nitrogen atom bonded thereto. The amide carbonate structure may be substituted, and examples of the substituent can include halogen (e.g., fluorine, chlorine, bromine, and iodine) and a hydroxyl group.

Examples of the amide derivative of the carboxyl group represented by R¹⁰ can include a carboxyl group in which the hydroxyl group is substituted by nitrogen bonded to 1 or 2 aforementioned linear or branched alkyl groups having 1 to 6 carbon atoms, linear or branched alkenyl groups having 2 to 6 carbon atoms, linear or branched alkynyl groups having 2 to 6 carbon atoms, alicyclic groups having 3 to 6 carbon atoms, aryl group having 5 to 10 carbon atoms, or the like. The amide derivative forms a urea structure together with the nitrogen atom bonded thereto. The urea structure may be substituted, and examples of the substituent can include halogen (e.g., fluorine, chlorine, bromine, and iodine) and a hydroxyl group.

Examples of the amino group represented by R¹⁰ can include an amino group, and an amino group in which 1 or 2 hydrogen atoms are substituted by 1 or 2 aforementioned linear or branched alkyl groups having 1 to 6 carbon atoms, linear or branched alkenyl groups having 2 to 6 carbon atoms, or linear or branched alkynyl groups having 2 to 6 carbon atoms. The substituted amino group may be further substituted, and examples of the substituent can include halogen (e.g., fluorine, chlorine, bromine, and iodine) and a hydroxyl group.

R¹⁰ mentioned above can be any of the groups listed above and is preferably hydrogen, a methyl group, an ethyl group, a n-propyl group, an isopropyl group, a cyclopentyl group, a cyclohexyl group, a phenyl group, a phenylmethyl group, a fluorophenyl group, a chlorophenyl group, a bromophenyl group, a difluorophenyl group, a dichlorophenyl group, a dibromophenyl group, a methylphenyl group, an ethylphenyl group, a n-propylphenyl group, an isopropylphenyl group, a methoxyphenyl group, an ethoxyphenyl group, a n-propylphenyl group, an isopropylphenyl group, a fluorophenylmethyl group, a chlorophenylmethyl group, a bromophenylmethyl group, a methoxyphenylmethyl group, an ethoxyphenylmethyl group, a n-propylphenylmethyl group, an isopropylphenylmethyl group, a methoxycarbonylphenyl group, a dimethoxycarbonylmethyl group, a pyridyl group, a pyridylmethyl group, a thiophenyl group, a thiophenemethyl group, a furan group, a furanylmethyl group, a pyrrole group, a pyrrolomethyl group, a carboxylmethyl group, a carboxylethyl group, a carboxylpropyl group, a cyano group, an amino group, a dimethylamino group, or a diethylamino group, more preferably hydrogen, a methyl group, an ethyl group, a cyclopentyl group, a cyclohexyl group, a phenyl group, a phenylmethyl group, a fluorophenyl group, a chlorophenyl group, a bromophenyl group, a methylphenyl group, an ethylphenyl group, a methoxyphenyl group, an ethoxyphenyl group, a fluorophenylmethyl group, a chlorophenylmethyl group, a bromophenylmethyl group, a methoxyphenylmethyl group, an ethoxyphenylmethyl group, a methoxycarbonylphenyl group, a dimethoxycarbonylmethyl group, a pyridyl group, a pyridylmethyl group, a thiophenyl group, a thiophenemethyl group, a cyano group, or a diethylamino group, further preferably hydrogen, a methyl group, an ethyl group, a cyclohexyl group, a phenyl group, a phenylmethyl group, a fluorophenyl group, a chlorophenyl group, a bromophenyl group, an ethylphenyl group, a methoxyphenyl group, a fluorophenylmethyl group, a methoxyphenylmethyl group, a dimethoxycarbonylmethyl group, a pyridylmethyl group, a thiophenemethyl group, or a carboxylmethyl group.

R¹⁰ mentioned above optionally forms a substituted or unsubstituted nitrogen-containing heterocyclic ring together with R¹¹ and the nitrogen atom bonded thereto.

R¹¹ in the formula (I) is any one group selected from hydrogen, a substituted or unsubstituted linear or branched alkyl group having 1 to 6 carbon atoms, a substituted or unsubstituted linear or branched alkenyl group having 2 to 6 carbon atoms, a substituted or unsubstituted linear or branched alkynyl group having 2 to carbon atoms, and a substituted or unsubstituted alicyclic group having 3 to 6 carbon atoms, or is a group that forms a substituted or unsubstituted nitrogen-containing heterocyclic ring together with R¹⁰ and the nitrogen atom bonded thereto.

Specific examples of the linear or branched alkyl group having 1 to 6 carbon atoms represented by R¹¹ can include a methyl group, an ethyl group, a propyl group, an isopropyl group, a n-butyl group, an isobutyl group, a sec-butyl group, a tert-butyl group, a n-pentyl group, a 1-methylbutyl group, a 2-methylbutyl group, a 3-methylbutyl group, a 1,1-dimethylpropyl group, a 1,2-dimethylpropyl group, a 2,2-dimethylpropyl group, a 3-pentyl group, a n-hexyl group, a 1-methylheptyl group, a 2-methylheptyl group, a 3-methylheptyl group, a 4-methylheptyl group, a 1,1-dimethylbutyl group, a 1,2-dimethylbutyl group, a 1,3-dimethylbutyl group, a 2,2-dimethylbutyl group, a 2,3-dimethylbutyl group, a 3,3-dimethylbutyl group, a 3,3-dimethylbutan-2-yl group, a 2,3-dimethylbutan-2-yl group, a 3-hexyl group, a 2-ethylpentyl group, and a 2-methylpentan-3-yl group.

Specific examples of the linear or branched alkenyl group having 2 to 6 carbon atoms represented by R¹¹ can include a vinyl group, a 1-propenyl group, an allyl group, an isopropenyl group, a 1-butenyl group, a 2-butenyl group, a 3-butenyl group, a 1,3-butanedienyl group, a 1-ethylvinyl group, a 1-methyl-1-propenyl group, a 2-methyl-1-propenyl group, a 2-methyl-2-propenyl group, a pentenyl group, a pentadienyl group, a hexenyl group, a hexadienyl group, and a hexatrienyl group.

Specific examples of the linear or branched alkynyl group having 2 to 6 carbon atoms represented by R¹¹ can include an ethynyl group, a 1-propynyl group, a 2-propynyl group, a 1-butynyl group, a 2-butynyl group, a 3-butynyl group, a 1,3-butanediynyl group, a 1-methyl-2-propynyl group, a pentynyl group, a pentadiynyl group, a hexynyl group, a hexadiynyl group, and a hexatriynyl group.

The linear or branched alkyl group having 1 to 6 carbon atoms, the linear or branched alkenyl group having 2 to 6 carbon atoms, and the linear or branched alkynyl group having 2 to 6 carbon atoms may each be substituted, and examples of the substituent can include halogen (e.g., fluorine, chlorine, bromine, and iodine), a hydroxyl group, a carboxyl group or an ester derivative thereof, a cyano group, an alicyclic group, and a nitrogen-containing heterocyclic group.

Specific examples of the alicyclic group having 3 to 6 carbon atoms represented by R¹¹ can include a cyclopropyl group, a cyclopropenyl group, a cyclobutyl group, a cyclobutenyl group, a cyclobutadienyl group, a cyclopentyl group, a cyclopentenyl group, a cyclopentadienyl group, a cyclohexyl group, a cyclohexenyl group, and a cyclohexadienyl group. The alicyclic group may be substituted, and examples of the substituent can include halogen (e.g., fluorine, chlorine, bromine, and iodine), a hydroxyl group, the aforementioned linear or branched alkyl group having 1 to 4 carbon atoms, linear or branched alkenyl group having 2 to 4 carbon atoms, and linear or branched alkynyl group having 2 to 4 carbon atoms. The alicyclic group may be ring-fused with an aryl group, a nitrogen-containing aromatic group, or a heterocyclic group.

R¹¹ mentioned above can be any of the groups listed above and is preferably hydrogen, a methyl group, an ethyl group, a n-propyl group, an isopropyl group, a vinyl group, a 1-propenyl group, an allyl group, an ethynyl group, a 1-propynyl group, a 2-propynyl group, a cyclopentyl group, or a cyclohexyl group, more preferably hydrogen, a methyl group, an ethyl group, a vinyl group, an allyl group, an ethynyl group, a 1-propynyl group, or a cyclopentyl group, further preferably hydrogen, a methyl group, or an ethyl group.

R¹⁰ mentioned above optionally forms a substituted or unsubstituted nitrogen-containing heterocyclic ring together with R¹¹ and the nitrogen atom bonded thereto.

Specific examples of the nitrogen-containing heterocyclic ring constituted by the aforementioned R¹⁰ and R¹¹ together with the nitrogen atom bonded thereto can include pyrrolidine, pyrazolidine, imidazolidine, pyrrole, pyrazole, imidazole, oxazole, isoxazole, thiazole, isothiazole, piperidine, hexahydropyridazine, hexahydropyrimidine, piperazine, hexahydrotriazine, oxazinane, morpholine, thiazinane, thiomorpholine, indole, isoindole, pyrrolopyridine, and purine. The nitrogen-containing heterocyclic ring may be substituted, and examples of the substituent can include halogen (e.g., fluorine, chlorine, bromine, and iodine), a hydroxyl group, the aforementioned linear or branched alkyl group having 1 to 6 carbon atoms, linear or branched alkenyl group having to 6 carbon atoms, linear or branched alkynyl group having 2 to 6 carbon atoms, a substituted or unsubstituted aryl group having 6 to 10 carbon atoms, a carboxyl group and an ester derivative or amide derivative thereof, a cyano group, and an amino group. The nitrogen-containing heterocyclic ring may be further substituted by a nitrogen-containing heterocyclic ring.

The nitrogen-containing heterocyclic ring constituted by the aforementioned R¹⁰ and R¹¹ together with the nitrogen atom bonded thereto can be any of the groups listed above and is preferably pyrrolidine, imidazolidine, pyrrole, imidazole, piperidine, piperidine having a substituent at position 4, piperazine, piperazine having a substituent at position 4, or morpholine, more preferably pyrrolidine, imidazole, piperidine, piperidine substituted at position 4 by a substituted or unsubstituted alkyl group having 1 to 6 carbon atoms, piperidine substituted at position 4 by the aforementioned nitrogen-containing heterocyclic ring, 4-hydroxypiperidine, 4-acylpiperazine, piperazine substituted at position 4 by a substituted or unsubstituted alkyl group having 1 to 6 carbon atoms, piperazine substituted at position 4 by a substituted or unsubstituted phenyl group, or morpholine, further preferably pyrrolidine, piperidine, 4-(2-hydroxyethyl)piperidine, 4-(piperidin-1-yl)piperidine, 4-[4-(4-methylpiperazin-1-yl)-piperidin-1-yl]piperidine, 4-hydroxypiperidine, 4-acetylpiperazine, 4-benzoylpiperazine, 4-methylpiperazine, 4-ethylpiperazine, 4-n-propylpiperazine, 4-isopropylpiperazine, 4-(2-methoxyethyl)piperazine, 4-diphenylmethylpiperazine, 4-di(4-fluorophenyl)piperazine, 4-phenylpiperazine, 4-(2-fluorophenyl)piperazine, or morpholine.

The compound represented by the formula (I) can be any compound as long as the compound satisfies the conditions mentioned above. Specific examples thereof can include the following compounds (I-1) to (I-61):

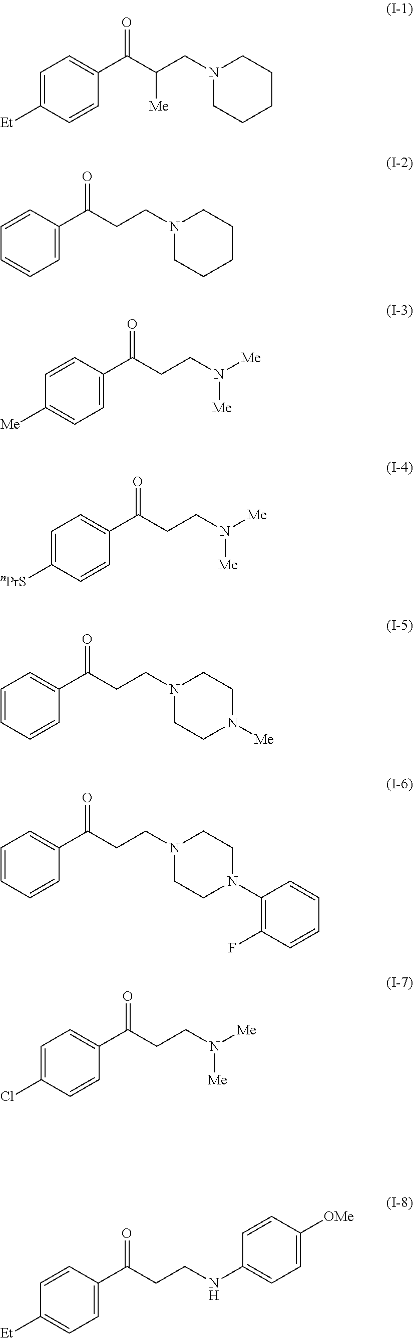

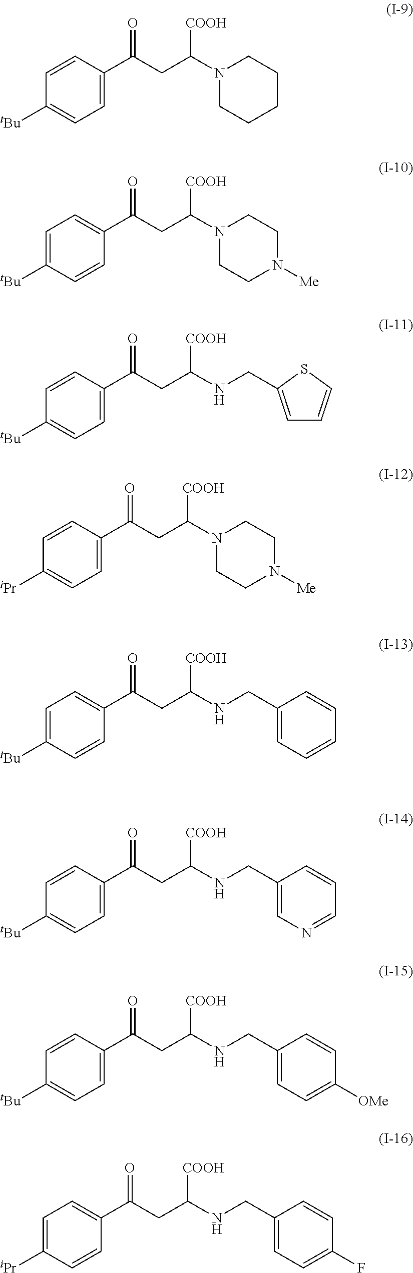

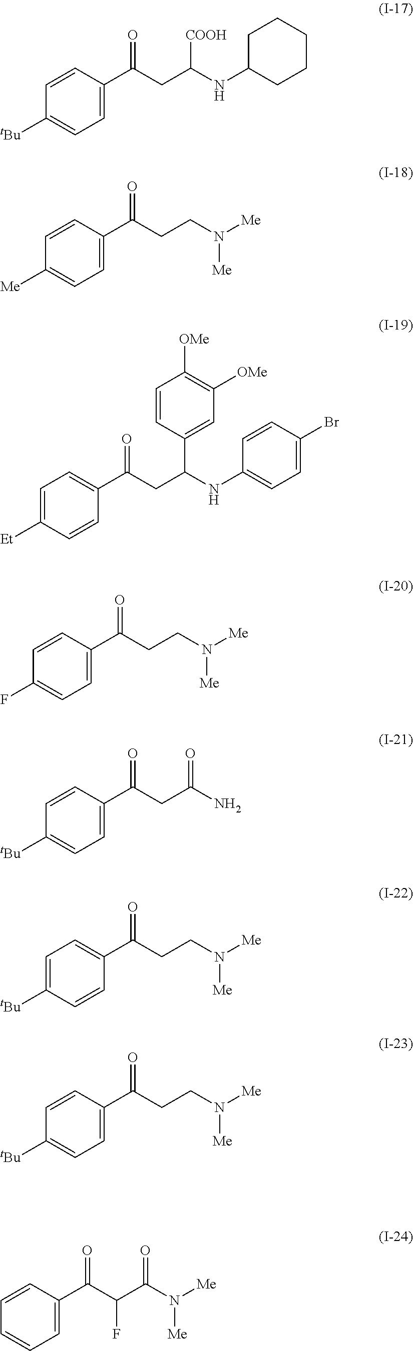

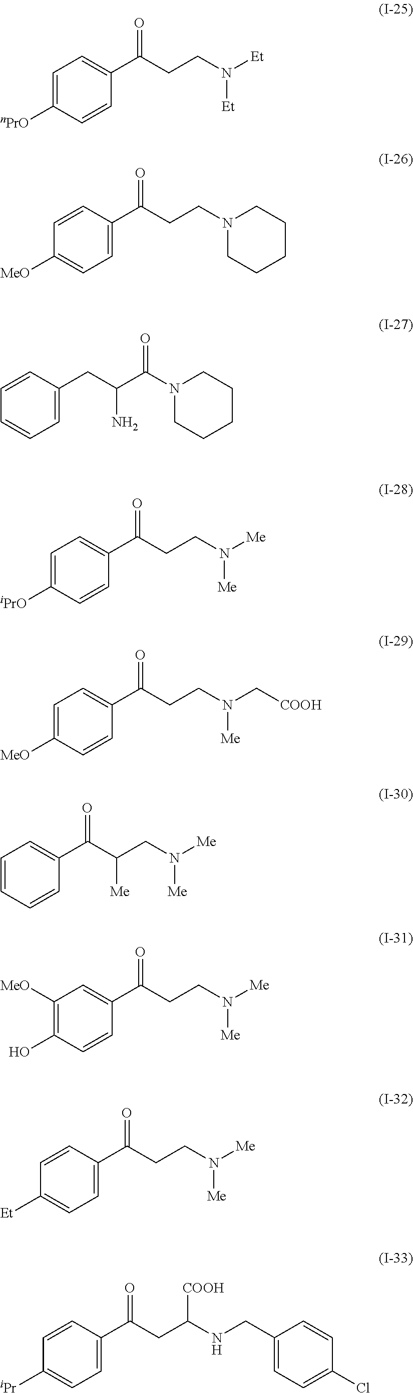

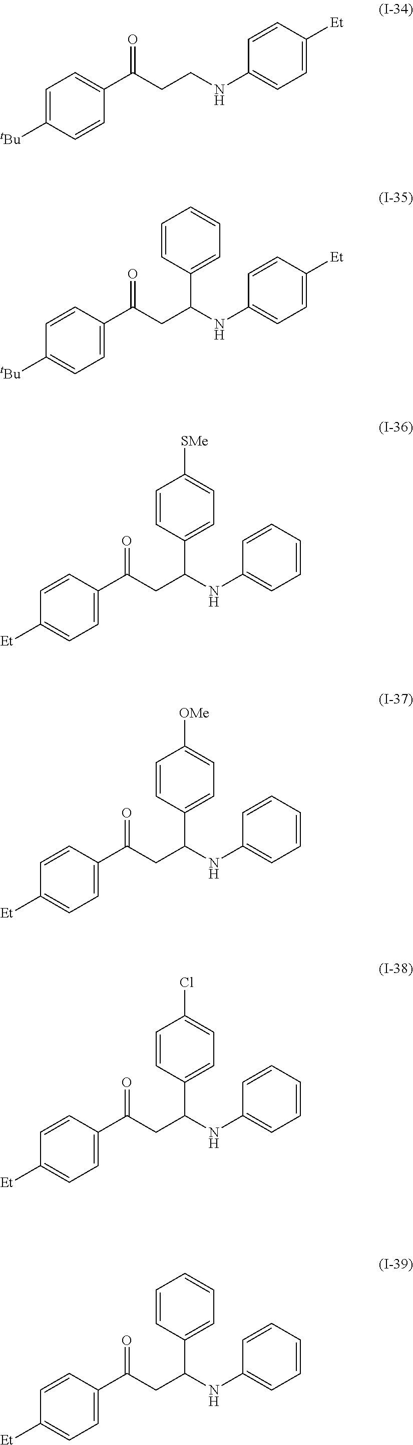 [SMILES: CCc1ccc(NCCC(=O)c2ccc(C(C)(C)C)cc2)cc1 ; CCc1ccc(NC(CC(=O)c2ccc(C(C)(C)C)cc2)c2ccccc2)cc1 ; CCc1ccc(C(=O)CC(Nc2ccccc2)c2ccc(SC)cc2)cc1 ; CCc1ccc(C(=O)CC(Nc2ccccc2)c2ccc(OC)cc2)cc1 ; CCc1ccc(C(=O)CC(Nc2ccccc2)c2ccc(Cl)cc2)cc1 ; CCc1ccc(C(=O)CC(Nc2ccccc2)c2ccccc2)cc1]

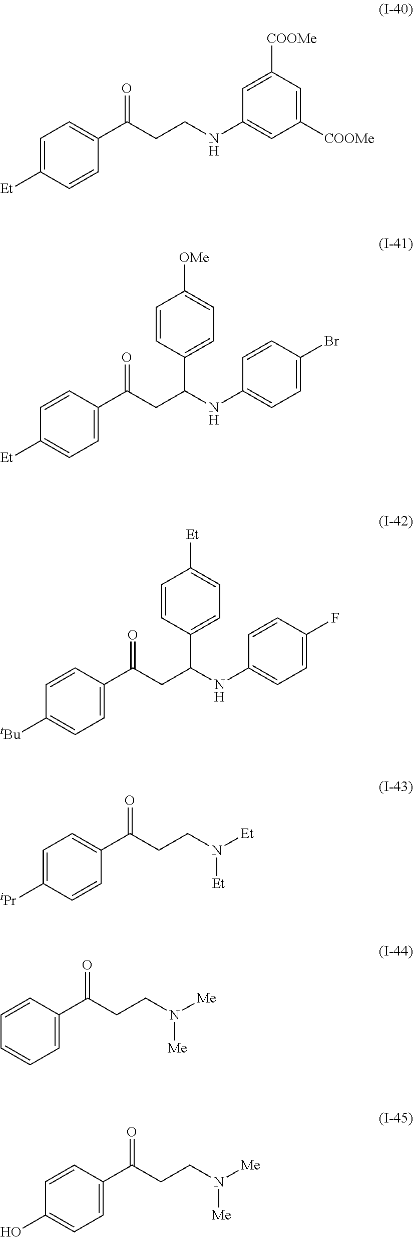 [SMILES: CCc1ccc(C(=O)CCNc2cc(C(=O)OC)cc(C(=O)OC)c2)cc1 ; CCc1ccc(C(=O)CC(Nc2ccc(Br)cc2)c2ccc(OC)cc2)cc1 ; CCc1ccc(C(CC(=O)c2ccc(C(C)(C)C)cc2)Nc2ccc(F)cc2)cc1 ; CCN(CC)CCC(=O)c1ccc(C(C)C)cc1 ; CN(C)CCC(=O)c1ccccc1 ; CN(C)CCC(=O)c1ccc(O)cc1]

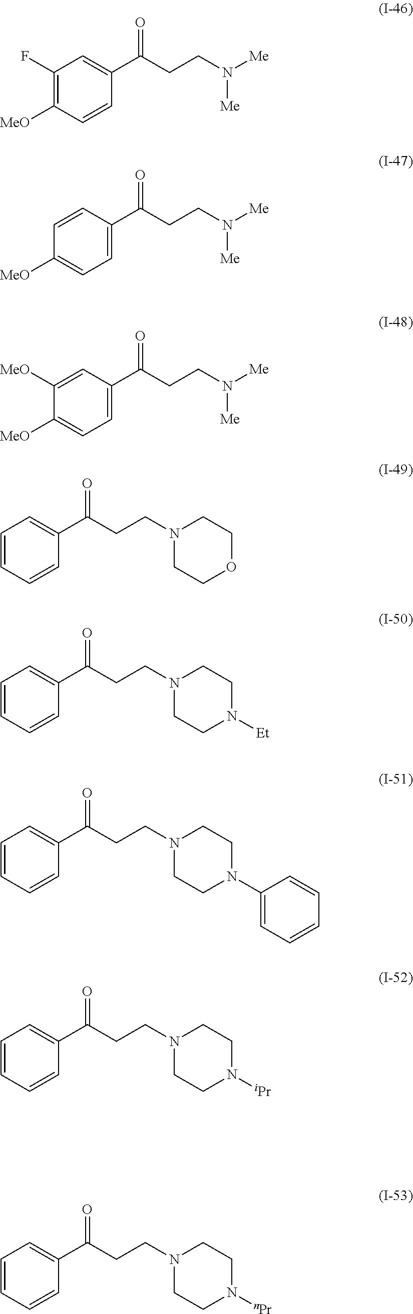

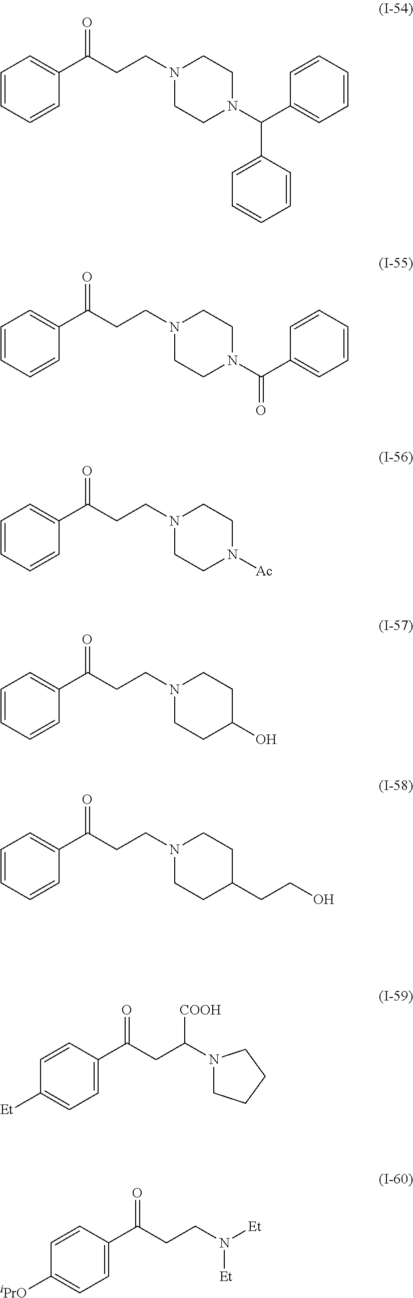

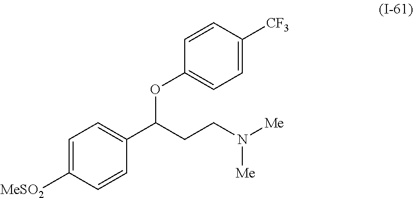

The compound represented by the formula (I) may be a compound represented by the following formula (IV):

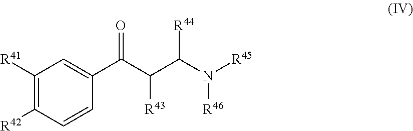

R⁴¹ in the formula (IV) is any one group selected from hydrogen, halogen, a hydroxyl group, a substituted or unsubstituted linear or branched alkyl group having 1 to 4 carbon atoms, a substituted or unsubstituted linear or branched alkenyl group having 2 to 4 carbon atoms, a substituted or unsubstituted linear or branched alkynyl group having 2 to 4 carbon atoms, and a substituted or unsubstituted linear or branched alkoxy group having 1 to 4 carbon atoms.

Specific examples of the linear or branched alkyl group having 1 to 4 carbon atoms represented by R⁴¹ can include a methyl group, an ethyl group, a propyl group, an isopropyl group, a n-butyl group, an isobutyl group, a sec-butyl group, and a tert-butyl group.

Specific examples of the linear or branched alkenyl group having 2 to 4 carbon atoms represented by R⁴¹ can include a vinyl group, a 1-propenyl group, an allyl group, an isopropenyl group, a 1-butenyl group, a 2-butenyl group, a 3-butenyl group, a 1,3-dibutenyl group, a 1-ethylvinyl group, a 1-methyl-1-propenyl group, a 2-methyl-1-propenyl group, and a 2-methyl-2-propenyl group.

Specific examples of the linear or branched alkynyl group having 2 to 4 carbon atoms represented by R⁴¹ can include an ethynyl group, a 1-propynyl group, a 2-propynyl group, a 1-butynyl group, a 2-butynyl group, a 3-butynyl group, a 1,3-dibutynyl group, and a 1-methyl-2-propynyl group.

The linear or branched alkyl group having 1 to 4 carbon atoms, the linear or branched alkenyl group having 2 to 4 carbon atoms, and the linear or branched alkynyl group having 2 to 4 carbon atoms may each be substituted, and examples of the substituent can include halogen (e.g., fluorine, chlorine, bromine, and iodine) and a hydroxyl group.

Examples of the substituted or unsubstituted linear or branched alkoxy group having 1 to 4 carbon atoms represented by R⁴¹ can include a group bonded via an oxygen atom to the aforementioned substituted or unsubstituted linear or branched alkyl group having 1 to 4 carbon atoms, substituted or unsubstituted linear or branched alkenyl group having 2 to 4 carbon atoms, or substituted or unsubstituted linear or branched alkynyl group having 2 to 4 carbon atoms.

R⁴¹ mentioned above can be any of the groups listed above and is preferably hydrogen, fluorine, chlorine, a methyl group, an ethyl group, a vinyl group, an ethynyl group, a methoxy group, or an ethoxy group, more preferably hydrogen, fluorine, an ethyl group, or a methoxy group, further preferably hydrogen, fluorine, or a methoxy group.

R⁴² in the formula (IV) is any one group selected from hydrogen, halogen, a hydroxyl group, a substituted or unsubstituted linear or branched alkyl group having 1 to 4 carbon atoms, a substituted or unsubstituted linear or branched alkenyl group having 2 to 4 carbon atoms, a substituted or unsubstituted linear or branched alkynyl group having 2 to 4 carbon atoms, and a substituted or unsubstituted linear or branched alkoxy group having 1 to 4 carbon atoms. Specific examples thereof can include the same group as R⁴¹ described above.

R⁴³ in the formula (IV) is any one group selected from hydrogen, halogen, a hydroxyl group, a substituted or unsubstituted linear or branched alkyl group having 1 to 4 carbon atoms, a substituted or unsubstituted linear or branched alkenyl group having 2 to 4 carbon atoms, a substituted or unsubstituted linear or branched alkynyl group having 2 to 4 carbon atoms, and a substituted or unsubstituted linear or branched alkoxy group having 1 to 4 carbon atoms. Specific examples thereof can include the same group as R⁴¹ described above.

R⁴⁴ in the formula (IV) is any one group selected from hydrogen, a carboxyl group, a substituted or unsubstituted linear or branched alkyl group having 1 to 4 carbon atoms, and a substituted or unsubstituted aryl group having 6 to 10 carbon atoms.

Specific examples of the linear or branched alkyl group having 1 to 4 carbon atoms represented by R⁴⁴ can include a methyl group, an ethyl group, a propyl group, an isopropyl group, a n-butyl group, an isobutyl group, a sec-butyl group, and a tert-butyl group. The alkyl group may be substituted, and examples of the substituent can include halogen (e.g., fluorine, chlorine, bromine, and iodine), a hydroxyl group, a carboxyl group or an ester derivative thereof, a cyano group, an alicyclic group, and a nitrogen-containing heterocyclic group.

Specific examples of the aryl group having 6 to 10 carbon atoms represented by R⁴⁴ can include a phenyl group and a naphthyl group. The aryl group may be substituted, and examples of the substituent can include halogen (e.g., fluorine, chlorine, bromine, and iodine), a hydroxyl group, the aforementioned linear or branched alkyl group having 1 to 4 carbon atoms, linear or branched alkenyl group having 2 to 4 carbon atoms, linear or branched alkynyl group having 2 to 4 carbon atoms, an alkoxy group substituted via an oxygen atom by the aforementioned linear or branched alkyl group having 1 to 4 carbon atoms, and a sulfide group substituted via a sulfur atom by the aforementioned linear or branched alkyl group having 1 to 4 carbon atoms. The aryl group may be ring-fused with an alicyclic group, a nitrogen-containing aromatic group, or a heterocyclic group.

R⁴⁴ mentioned above can be any of the groups listed above and is preferably hydrogen, fluorine, chlorine, bromine, iodine, a methyl group, an ethyl group, a phenyl group, a tolyl group, an ethylphenyl group, a hydroxyphenyl group, a methoxyphenyl group, a dimethoxyphenyl group, an ethoxyphenyl group, a fluorophenyl group, a chlorophenyl group, a methylthiophenyl group, an ethylthiophenyl group, a naphthyl group, or a carboxyl group, more preferably hydrogen, fluorine, a methyl group, an ethyl group, a phenyl group, a tolyl group, an ethylphenyl group, a hydroxyphenyl group, a methoxyphenyl group, a dimethoxyphenyl group, an ethoxyphenyl group, a fluorophenyl group, a chlorophenyl group, a methylthiophenyl group, an ethylthiophenyl group, a naphthyl group, or a carboxyl group, further preferably hydrogen, fluorine, a phenyl group, an ethylphenyl group, a methoxyphenyl group, a dimethoxyphenyl group, a chlorophenyl group, a methylthiophenyl group, or a carboxyl group.

R⁴⁵ in the formula (IV) is any one group selected from hydrogen, a substituted or unsubstituted linear or branched alkyl group having 1 to 4 carbon atoms, a substituted or unsubstituted linear or branched alkenyl group having 2 to 4 carbon atoms, and a substituted or unsubstituted linear or branched alkynyl group having 2 to 4 carbon atoms.

Specific examples of the linear or branched alkyl group having 1 to 4 carbon atoms represented by R⁴⁵ can include a methyl group, an ethyl group, a propyl group, an isopropyl group, a n-butyl group, an isobutyl group, a sec-butyl group, and a tert-butyl group.

Specific examples of the linear or branched alkenyl group having 2 to 4 carbon atoms represented by R⁴⁵ can include a vinyl group, a 1-propenyl group, an allyl group, an isopropenyl group, a 1-butenyl group, a 2-butenyl group, a 3-butenyl group, a 1,3-dibutenyl group, a 1-ethylvinyl group, a 1-methyl-1-propenyl group, a 2-methyl-1-propenyl group, and a 2-methyl-2-propenyl group.

Specific examples of the linear or branched alkynyl group having 2 to 4 carbon atoms represented by R⁴⁵ can include an ethynyl group, a 1-propynyl group, a 2-propynyl group, a 1-butynyl group, a 2-butynyl group, a 3-butynyl group, a 1,3-dibutynyl group, and a 1-methyl-2-propynyl group.

The linear or branched alkyl group having 1 to 4 carbon atoms, the linear or branched alkenyl group having 2 to 4 carbon atoms, and the linear or branched alkynyl group having 2 to 4 carbon atoms may each be substituted, and examples of the substituent can include halogen (e.g., fluorine, chlorine, bromine, and iodine) and a hydroxyl group.

R⁴⁵ mentioned above can be any of the groups listed above and is preferably hydrogen, a methyl group, an ethyl group, a vinyl group, or an ethynyl group, more preferably hydrogen, a methyl group, or an ethyl group.

R⁴⁵ mentioned above optionally forms a substituted or unsubstituted nitrogen-containing heterocyclic ring together with R⁴⁶ and the nitrogen atom bonded thereto.

R⁴⁶ in the formula (IV) is any one group selected from hydrogen, a substituted or unsubstituted linear or branched alkyl group having 1 to 4 carbon atoms, a substituted or unsubstituted linear or branched alkenyl group having 2 to 4 carbon atoms, a substituted or unsubstituted linear or branched alkynyl group having 2 to 4 carbon atoms, a substituted or unsubstituted aryl group having 5 to 10 carbon atoms, and an arylalkyl group consisting of a substituted or unsubstituted linear or branched alkyl group having 1 to 4 carbon atoms and a substituted or unsubstituted aryl group having 5 to 10 carbon atoms.

Specific examples of the linear or branched alkyl group having 1 to 4 carbon atoms represented by R⁴⁶ can include a methyl group, an ethyl group, a propyl group, an isopropyl group, a n-butyl group, an isobutyl group, a sec-butyl group, and a tert-butyl group.

Specific examples of the linear or branched alkenyl group having 2 to 4 carbon atoms represented by R⁴⁶ can include a vinyl group, a 1-propenyl group, an allyl group, an isopropenyl group, a 1-butenyl group, a 2-butenyl group, a 3-butenyl group, a 1,3-dibutenyl group, a 1-ethylvinyl group, a 1-methyl-1-propenyl group, a 2-methyl-1-propenyl group, and a 2-methyl-2-propenyl group.

Specific examples of the linear or branched alkynyl group having 2 to 4 carbon atoms represented by R⁴⁶ can include an ethynyl group, a 1-propynyl group, a 2-propynyl group, a 1-butynyl group, a 2-butynyl group, a 3-butynyl group, a 1,3-dibutynyl group, and a 1-methyl-2-propynyl group.

The linear or branched alkyl group having 1 to 4 carbon atoms, the linear or branched alkenyl group having 2 to 4 carbon atoms, and the linear or branched alkynyl group having 2 to 4 carbon atoms may each be substituted, and examples of the substituent can include halogen (e.g., fluorine, chlorine, bromine, and iodine), a hydroxyl group, and a carboxyl group.

Specific examples of the aryl group having 5 to 10 carbon atoms represented by R⁴⁶ can include a phenyl group, a pyridyl group, and a naphthyl group. The aryl group may be substituted, and examples of the substituent can include halogen (e.g., fluorine, chlorine, bromine, and iodine), a hydroxyl group, the aforementioned linear or branched alkyl group having 1 to 4 carbon atoms, linear or branched alkenyl group having 2 to 4 carbon atoms, linear or branched alkynyl group having 2 to 4 carbon atoms, an alkoxy group substituted via an oxygen atom by the aforementioned linear or branched alkyl group having 1 to carbon atoms, and a sulfide group substituted via a sulfur atom by the aforementioned linear or branched alkyl group having 1 to 4 carbon atoms. The aryl group may be ring-fused with an alicyclic group, a nitrogen-containing aromatic group, or a heterocyclic group.

The arylalkyl group consisting of a substituted or unsubstituted linear or branched alkyl group having 1 to 4 carbon atoms and a substituted or unsubstituted aryl group having 5 to 10 carbon atoms, represented by R⁴⁶ is a group in which the aforementioned substituted or unsubstituted linear or branched alkyl group having 1 to 4 carbon atoms is substituted by the aforementioned substituted or unsubstituted aryl group having 5 to 10 carbon atoms.

R⁴⁶ mentioned above can be any of the groups listed above and is preferably hydrogen, a methyl group, an ethyl group, a n-propyl group, a phenyl group, a fluorophenyl group, a chlorophenyl group, a bromophenyl group, a difluorophenyl group, a dichlorophenyl group, a dibromophenyl group, a methylphenyl group, an ethylphenyl group, a n-propylphenyl group, an isopropylphenyl group, a methoxyphenyl group, an ethoxyphenyl group, a phenylmethyl group, a fluorophenylmethyl group, a chlorophenylmethyl group, a bromophenylmethyl group, a methoxyphenylmethyl group, an ethoxyphenylmethyl group, a n-propylphenylmethyl group, an isopropylphenylmethyl group, a pyridyl group, a pyridylmethyl group, or a carboxylmethyl group, more preferably hydrogen, a methyl group, an ethyl group, a phenyl group, a fluorophenyl group, a chlorophenyl group, a bromophenyl group, a methylphenyl group, a ethylphenyl group, a methoxyphenyl group, an ethoxyphenyl group, a phenylmethyl group, a fluorophenylmethyl group, a chlorophenylmethyl group, a bromophenylmethyl group, a methoxyphenylmethyl group, an ethoxyphenylmethyl group, a pyridyl group, or a pyridylmethyl group, further preferably hydrogen, a methyl group, an ethyl group, a phenyl group, a fluorophenyl group, a chlorophenyl group, a bromophenyl group, an ethylphenyl group, a methoxyphenyl group, a phenylmethyl group, a fluorophenylmethyl group, a methoxyphenylmethyl group, or a pyridylmethyl group.

R⁴⁶ mentioned above optionally forms a substituted or unsubstituted nitrogen-containing heterocyclic ring together with R⁴⁵ and the nitrogen atom bonded thereto.

Specific examples of the nitrogen-containing heterocyclic ring constituted by the aforementioned R⁴⁵ and R⁴⁶ together with the nitrogen atom bonded thereto can include pyrrolidine, pyrazolidine, imidazolidine, pyrrole, pyrazole, imidazole, oxazole, isoxazole, thiazole, isothiazole, piperidine, hexahydropyridazine, hexahydropyrimidine, piperazine, hexahydrotriazine, oxazinane, morpholine, thiazinane, thiomorpholine, indole, isoindole, pyrrolopyridine, and purine. The nitrogen-containing heterocyclic ring may be substituted, and examples of the substituent can include halogen (e.g., fluorine, chlorine, bromine, and iodine), a hydroxyl group, the aforementioned linear or branched alkyl group having 1 to 6 carbon atoms, linear or branched alkenyl group having 2 to 6 carbon atoms, linear or branched alkynyl group having 2 to 6 carbon atoms, a substituted or unsubstituted aryl group having 6 to 10 carbon atoms, a carboxyl group and an ester derivative or an amide derivative thereof, a cyano group, and an amino group. The nitrogen-containing heterocyclic ring may be further substituted by a nitrogen-containing heterocyclic ring.

The nitrogen-containing heterocyclic ring constituted by the aforementioned R⁴⁵ and R⁴⁶ together with the nitrogen atom bonded thereto can be any of the groups listed above and is preferably pyrrolidine, imidazolidine, pyrrole, imidazole, piperidine, piperidine having a substituent at position 4, piperazine, piperazine having a substituent at position 4, or morpholine, more preferably pyrrolidine, imidazole, piperidine, piperidine substituted at position 4 by a substituted or unsubstituted alkyl group having 1 to 6 carbon atoms, piperidine substituted at position 4 by the aforementioned nitrogen-containing heterocyclic ring, 4-hydroxypiperidine, 4-acylpiperazine, piperazine substituted at position 4 by a substituted or unsubstituted alkyl group having 1 to 6 carbon atoms, piperazine substituted at position 4 by a substituted or unsubstituted phenyl group, or morpholine, further preferably pyrrolidine, piperidine, 4-(2-hydroxyethyl)piperidine, 4-(piperidin-1-yl)piperidine, 4-[4-(4-methylpiperazin-1-yl)-piperidin-1-yl]piperidine, 4-hydroxypiperidine, 4-acetylpiperazine, 4-benzoylpiperazine, 4-methylpiperazine, 4-ethylpiperazine, 4-n-propylpiperazine, 4-isopropylpiperazine, 4-(2-methoxyethyl)piperazine, 4-diphenylmethylpiperazine, 4-di(4-fluorophenyl)piperazine, 4-phenylpiperazine, 4-(2-fluorophenyl)piperazine, or morpholine.

The compound represented by the formula (IV) can be any compound as long as the compound satisfies the conditions mentioned above. Specific examples thereof can include the following compounds (IV-1) to (IV-4):

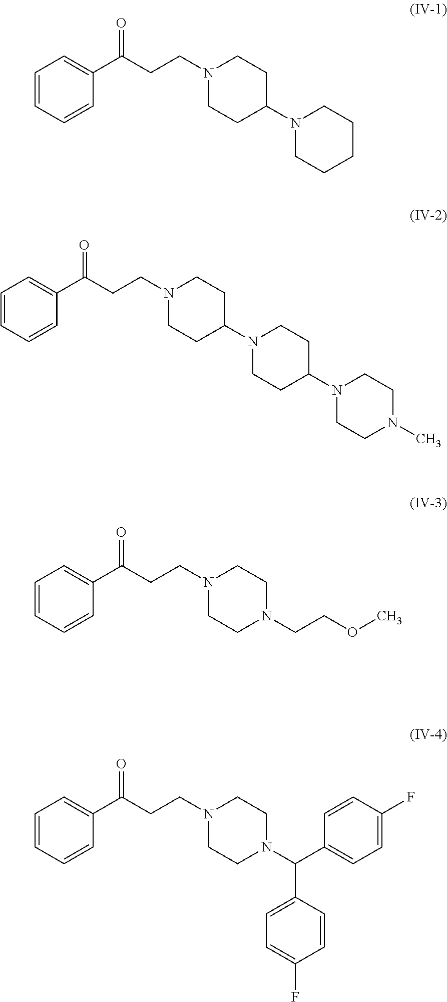 [SMILES: O=C(CCN1CCC(N2CCCCC2)CC1)c1ccccc1 ; CN1CCN(C2CCN(C3CCN(CCC(=O)c4ccccc4)CC3)CC2)CC1 ; COCCN1CCN(CCC(=O)c2ccccc2)CC1 ; O=C(CCN1CCN(C(c2ccc(F)cc2)c2ccc(F)cc2)CC1)c1ccccc1]

R²¹ in the formula (II) is hydrogen, a substituted or unsubstituted aryl group having 6 to 10 carbon atoms, or a substituted or unsubstituted deuterated aryl group having 6 to 10 carbon atoms. Specific examples of the aryl group can include a phenyl group and a naphthyl group. The deuterated aryl group refers to the aforementioned phenyl group, naphthyl group, or the like in which one or more hydrogen atoms on carbon are substituted by deuterium. Examples of the substituent for the aryl group or the deuterated aryl group can include halogen (e.g., fluorine, chlorine, bromine, and iodine), a substituted or unsubstituted linear or branched alkyl group having 1 to 4 carbon atoms, a substituted or unsubstituted linear or branched alkenyl group having 2 to 4 carbon atoms, a substituted or unsubstituted linear or branched alkynyl group having 2 to 4 carbon atoms, a substituted or unsubstituted linear or branched alkoxy group having 1 to 4 carbon atoms, a substituted or unsubstituted linear or branched alkenyloxy group having 2 to 4 carbon atoms, and a substituted or unsubstituted linear or branched alkynyloxy group having 2 to 4 carbon atoms. These substituents may each be further substituted by one or more deuterium atoms, halogen atoms, hydroxyl groups, or the like.

R²¹ mentioned above can be any of the groups listed above and is preferably hydrogen, a phenyl group, a penta-deuterated phenyl group, a p-tolyl group, a 1-naphthyl group, or a 2-naphthyl group, more preferably hydrogen, a phenyl group, or a penta-deuterated phenyl group, most preferably hydrogen or a phenyl group.

R²² in the formula (II) is any one group selected from a substituted or unsubstituted linear or branched alkyl group having 1 to 4 carbon atoms, a substituted or unsubstituted linear or branched deuterated alkyl group having 1 to 4 carbon atoms, a substituted or unsubstituted linear or branched alkenyl group having 2 to 4 carbon atoms, a substituted or unsubstituted linear or branched deuterated alkenyl group having 2 to 4 carbon atoms, a substituted or unsubstituted linear or branched alkynyl group having 2 to 4 carbon atoms, and a substituted or unsubstituted linear or branched deuterated alkynyl group having 2 to 4 carbon atoms.

Specific examples of the linear or branched alkyl group having 1 to 4 carbon atoms represented by R²² can include a methyl group, an ethyl group, a propyl group, an isopropyl group, a n-butyl group, an isobutyl group, a sec-butyl group, and a tert-butyl group.

Specific examples of the linear or branched alkenyl group having 2 to 4 carbon atoms represented by R²² can include a vinyl group, a 1-propenyl group, an allyl group, an isopropenyl group, a 1-butenyl group, a 2-butenyl group, a 3-butenyl group, a 1,3-dibutenyl group, a 1-ethylvinyl group, a 1-methyl-1-propenyl group, a 2-methyl-1-propenyl group, and a 2-methyl-2-propenyl group.

Specific examples of the linear or branched alkynyl group having 2 to 4 carbon atoms represented by R²² can include an ethynyl group, a 1-propynyl group, a 2-propynyl group, a 1-butynyl group, a 2-butynyl group, a 3-butynyl group, a 1,3-dibutynyl group, and a 1-methyl-2-propynyl group.

Examples of the linear or branched deuterated alkyl group having 1 to 4 carbon atoms, the linear or branched deuterated alkenyl group having 2 to 4 carbon atoms, and the linear or branched deuterated alkynyl group having 2 to 4 carbon atoms represented by R²² can include the aforementioned linear or branched alkyl group having 1 to 4 carbon atoms, linear or branched alkenyl group having 2 to 4 carbon atoms, and linear or branched alkynyl group having 2 to 4 carbon atoms, in which one or more hydrogen atoms on carbon are substituted by deuterium.

The linear or branched alkyl group having 1 to 4 carbon atoms, the linear or branched deuterated alkyl group having 1 to 4 carbon atoms, the linear or branched alkenyl group having 2 to 4 carbon atoms, the substituted or unsubstituted linear or branched deuterated alkenyl group having 2 to 4 carbon atoms, the substituted or unsubstituted linear or branched alkynyl group having 2 to 4 carbon atoms, and the substituted or unsubstituted linear or branched deuterated alkynyl group having 2 to 4 carbon atoms may each be substituted, and examples of the substituent can include halogen (e.g., fluorine, chlorine, bromine, and iodine) and a hydroxyl group.

R²² mentioned above can be any of the groups listed above and is preferably, specifically, a methyl group, a tri-deuterated methyl group, an ethyl group, a penta-deuterated ethyl group, a vinyl group, or an ethynyl group, more preferably a methyl group or a tri-deuterated methyl group, most preferably a methyl group.

R²³ in the formula (II) is any one group selected from hydrogen, a substituted or unsubstituted linear or branched alkyl group having 1 to 4 carbon atoms, a substituted or unsubstituted linear or branched deuterated alkyl group having 1 to 4 carbon atoms, a substituted or unsubstituted linear or branched alkenyl group having 2 to 4 carbon atoms, a substituted or unsubstituted linear or branched deuterated alkenyl group having 2 to 4 carbon atoms, a substituted or unsubstituted linear or branched alkynyl group having 2 to 4 carbon atoms, and a substituted or unsubstituted linear or branched deuterated alkynyl group having 2 to 4 carbon atoms.

Specific examples of the linear or branched alkyl group having 1 to 4 carbon atoms represented by R²³ can include a methyl group, an ethyl group, a propyl group, an isopropyl group, a n-butyl group, an isobutyl group, a sec-butyl group, and a tert-butyl group.

Examples of the linear or branched alkenyl group having 2 to 4 carbon atoms represented by R²³ can include a vinyl group, a 1-propenyl group, an allyl group, an isopropenyl group, a 1-butenyl group, a 2-butenyl group, a 3-butenyl group, a 1,3-dibutenyl group, a 1-ethylvinyl group, a 1-methyl-1-propenyl group, a 2-methyl-1-propenyl group, and a 2-methyl-2-propenyl group.

Specific examples of the linear or branched alkynyl group having 2 to 4 carbon atoms represented by R²³ can include an ethynyl group, a 1-propynyl group, a 2-propynyl group, a 1-butynyl group, a 2-butynyl group, a 3-butynyl group, a 1,3-dibutynyl group, and a 1-methyl-2-propynyl group.

Examples of the substituted or unsubstituted linear or branched deuterated alkyl group having 1 to 4 carbon atoms, the substituted or unsubstituted linear or branched deuterated alkenyl group having 2 to 4 carbon atoms, and the substituted or unsubstituted linear or branched deuterated alkynyl group having 2 to 4 carbon atoms represented by R²³ can include the aforementioned linear or branched alkyl group having 1 to 4 carbon atoms, linear or branched alkenyl group having 2 to 4 carbon atoms, and linear or branched alkynyl group having 2 to 4 carbon atoms, in which one or more hydrogen atoms on carbon is substituted by deuterium.

The substituted or unsubstituted linear or branched alkyl group having 1 to 4 carbon atoms, the substituted or unsubstituted linear or branched deuterated alkyl group having 1 to 4 carbon atoms, the substituted or unsubstituted linear or branched alkenyl group having 2 to 4 carbon atoms, the substituted or unsubstituted linear or branched deuterated alkenyl group having 2 to 4 carbon atoms, the substituted or unsubstituted linear or branched alkynyl group having 2 to 4 carbon atoms, and the substituted or unsubstituted linear or branched deuterated alkynyl group having 2 to 4 carbon atoms may each be substituted, and examples of the substituent can include halogen (e.g., fluorine, chlorine, bromine, and iodine) and a hydroxyl group.

R²³ mentioned above can be any of the groups listed above and is preferably hydrogen, a methyl group, a tri-deuterated methyl group, an ethyl group, a penta-deuterated ethyl group, a vinyl group, or an ethynyl group, more preferably hydrogen, a methyl group, or a tri-deuterated methyl group, most preferably hydrogen or a methyl group.

X in the formula (II) is methylene or deuterated methylene. The deuterated methylene refers to methylene in which 1 or 2 hydrogen atoms on carbon are substituted by deuterium. Of them, methylene is preferred as X.

The compound represented by the formula (IV) can be any compound as long as the compound satisfies the conditions mentioned above. Specific examples thereof can include the following compounds (II-1) and (II-2):

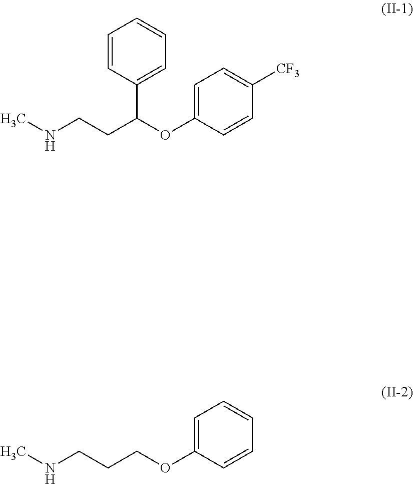 [SMILES: CNCCC(Oc1ccc(C(F)(F)F)cc1)c1ccccc1 ; CNCCCOc1ccccc1]

R³¹ in the formula (III) is any one group selected from a substituted or unsubstituted linear or branched alkyl group having 1 to 4 carbon atoms, a substituted or unsubstituted linear or branched deuterated alkyl group having 1 to 4 carbon atoms, a substituted or unsubstituted linear or branched alkenyl group having 2 to 4 carbon atoms, a substituted or unsubstituted linear or branched deuterated alkenyl group having 2 to 4 carbon atoms, a substituted or unsubstituted linear or branched alkynyl group having 2 to 4 carbon atoms, a substituted or unsubstituted linear or branched deuterated alkynyl group having 2 to 4 carbon atoms, a substituted or unsubstituted heterocyclic group having a 5- or 6-membered ring, a substituted or unsubstituted aromatic group having 6 to 10 carbon atoms, a substituted or unsubstituted nitrogen-containing aromatic group having 2 to 10 carbon atoms, a sulfonic acid group, and a sulfonyl group.

Specific examples of the linear or branched alkyl group having 1 to 4 carbon atoms represented by R³¹ can include a methyl group, an ethyl group, a propyl group, an isopropyl group, a n-butyl group, an isobutyl group, a sec-butyl group, and a tert-butyl group.

Specific examples of the linear or branched alkenyl group having 2 to 4 carbon atoms represented by R³¹ can include a vinyl group, a 1-propenyl group, an allyl group, an isopropenyl group, a 1-butenyl group, a 2-butenyl group, a 3-butenyl group, a 1,3-dibutenyl group, a 1-ethylvinyl group, a 1-methyl-1-propenyl group, a 2-methyl-1-propenyl group, and a 2-methyl-2-propenyl group.

Specific examples of the linear or branched alkynyl group having 2 to 4 carbon atoms represented by R³¹ can include an ethynyl group, a 1-propynyl group, a 2-propynyl group, a 1-butynyl group, a 2-butynyl group, a 3-butynyl group, a 1,3-dibutynyl group, and a 1-methyl-2-propynyl group.

The linear or branched alkyl group having 1 to 4 carbon atoms, the linear or branched alkenyl group having 2 to 4 carbon atoms, and the linear or branched alkynyl group having 2 to 4 carbon atoms may each be substituted, and examples of the substituent can include halogen (e.g., fluorine, chlorine, bromine, and iodine), a hydroxyl group, a carboxyl group, a phenyl group, a pyridyl group, and a heterocyclic group having a 5- or 6-membered ring. These substituents may each be further substituted by the aforementioned linear or branched alkyl group having 1 to 4 carbon atoms, a substituted or unsubstituted amino group, the aforementioned linear or branched acyl group having 1 to 4 carbon atoms, the aforementioned linear or branched sulfonyl group having 1 to 4 carbon atoms, or the like.

Examples of the substituted or unsubstituted linear or branched deuterated alkyl group having 1 to 4 carbon atoms, the substituted or unsubstituted linear or branched deuterated alkenyl group having 2 to 4 carbon atoms, and the substituted or unsubstituted linear or branched deuterated alkynyl group having 2 to 4 carbon atoms represented by R³¹ can include the aforementioned substituted or unsubstituted linear or branched alkyl group having 1 to 4 carbon atoms, substituted or unsubstituted linear or branched alkenyl group having 2 to 4 carbon atoms, and substituted or unsubstituted linear or branched alkynyl group having 2 to 4 carbon atoms, in which one or more hydrogen atoms on carbon are substituted by deuterium.

Specific examples of the heterocyclic group having a 5- or 6-membered ring represented by R³¹ can include a pyrrolidinyl group, a pyrrolyl group, a tetrahydrofuranyl group, a furanyl group, a tetrahydrothiophenyl group, a thiophenyl group, an imidazolyl group, a pyrazolyl group, an oxazolyl group, a thiazolyl group, an imidazolinyl group, a piperidinyl group, a tetrahydropyranyl group, a tetrahydrothiopyranyl group, a piperazinyl group, a morpholino group, a thiazinyl group, and a thioxanyl group.

The heterocyclic group may be substituted, and examples of the substituent can include halogen (e.g., fluorine, chlorine, bromine, and iodine), a hydroxyl group, an amino group, a carboxyl group, the aforementioned linear or branched alkyl group having 1 to 4 carbon atoms, the aforementioned linear or branched alkenyl group having 2 to 4 carbon atoms, the alkynyl group having 2 to 4 carbon atoms, and a sulfonyl group. These nitrogen-containing heterocyclic groups may each be carbonylated at 1 or 2 carbon atoms in the ring and may each be ring-fused with 1 or 2 benzene rings. The carbonylation refers to the replacement of CH₂ with C═O.

Specific examples of the aromatic group having 6 to carbon atoms represented by R³¹ can include a phenyl group and a naphthyl group. The aromatic group may be substituted, and examples of the substituent can include halogen (e.g., fluorine, chlorine, bromine, and iodine), a hydroxyl group, a nitro group, an aminocarbonyl group, an acetyl group, and a cyano group.

Specific examples of the nitrogen-containing aromatic group having 2 to 10 carbon atoms represented by R³¹ can include a pyrrolyl group, a pyrazolyl group, an imidazolyl group, an isoxazolyl group, an oxazolyl group, an oxadiazolyl group, an isothiazolyl group, a thiazolyl group, a thiadiazolyl group, a pyridinyl group, a pyridazinyl group, a pyrimidinyl group, a pyrazinyl group, a triazinyl group, an indolyl group, an isoindolyl group, a benzoxazolyl group, a benzothiazolyl group, a benzisoxazolyl group, a benzisothiazolyl group, a benzoxadiazolyl group, a benzothiadiazolyl group, a pyrrolopyridinyl group, a pyrrolopyrazinyl group, a purinyl group, a quinolinyl group, an isoquinolinyl group, a cinnolinyl group, a quinazolinyl group, and a quinoxalinyl group.

The nitrogen-containing aromatic group having 2 to 10 carbon atoms may be substituted, and examples of the substituent can include halogen (e.g., fluorine, chlorine, bromine, and iodine), a hydroxyl group, a nitro group, an aminocarbonyl group, an acetyl group, and a cyano group.

Specific examples of the sulfonyl group represented by R³¹ can include a methanesulfonyl group, a trifluoromethanesulfonyl group, an ethanesulfonyl group, a butanesulfonyl group, a benzenesulfonyl group, a toluenesulfonyl group, a mesitylenesulfonyl group, a naphthalenesulfonyl group, and a camphorsulfonyl group.

The sulfonyl group may be substituted, and examples of the substituent can include halogen (e.g., fluorine, chlorine, bromine, and iodine), a hydroxyl group, a nitro group, and a cyano group.

R³¹ mentioned above can be any of the groups listed above and is preferably a methyl group, an ethyl group, a vinyl group, an ethynyl group, an isopropyl group, an isopropenyl group, a tri-deuterated methyl group, a penta-deuterated ethyl group, a hepta-deuterated isopropyl group, a 1,1,1,3,3,3-hexa-deuterated isopropyl group, a trifluoromethyl group, a benzyl group, a hydroxybenzyl group, a pyridylmethyl group, a pyrrolidinylmethyl group, a piperidinylmethyl group, a morpholinomethyl group, a piperazinylmethyl group, a (N-methyl-piperazinyl)methyl group, an acetamidomethyl group, a 2-pyrrolidinylethyl group, a 2-piperidinylethyl group, a 2-morpholinoethyl group, a 2-piperazinylethyl group, a 2-(N-methyl-piperazinyl)ethyl group, a 2-acetamidoethyl group, a pyrrolidinyl group, a piperidyl group, a hydroxypiperidyl group, a N-methylpiperidyl group, a N-acetylpiperidyl group, a N-methylsulfonylpiperidyl group, a tetrahydrofuranyl group, a tetrahydrothiopyranyl group, a phenyl group, a tolyl group, a hydroxyphenyl group, a carboxylphenyl group, an aminocarbonylphenyl group, a pyridyl group, or a sulfonyl group, more preferably a methyl group, an ethyl group, an isopropyl group, a tri-deuterated methyl group, a trifluoromethyl group, a hydroxybenzyl group, a pyridylmethyl group, a 2-pyrrolidinylethyl group, a 2-piperidinylethyl group, a 2-morpholinoethyl group, a 2-piperazinylethyl group, a 2-(N-methyl-piperazinyl)ethyl group, an acetamidomethyl group, a 2-acetamidoethyl group, a pyrrolidinyl group, a piperidyl group, a hydroxypiperidyl group, a N-methylpiperidyl group, a N-acetylpiperidyl group, a N-methylsulfonylpiperidyl group, a tetrahydrofuranyl group, a tetrahydrothiopyranyl group, a carboxylphenyl group, an aminocarbonylphenyl group, a pyridyl group, or a sulfonyl group, most preferably a methyl group.

R³² in the formula (III) is hydrogen, a hydroxymethyl group or a hydroxymethyl group in which hydrogen on carbon is substituted by deuterium. Of them, a hydroxymethyl group is preferred as R³².

R³³ in the formula (III) is any one group selected from a substituted or unsubstituted linear or branched alkyl group having 1 to 6 carbon atoms, a substituted or unsubstituted linear or branched alkenyl group having 2 to 6 carbon atoms, a substituted or unsubstituted linear or branched alkynyl group having 2 to 6 carbon atoms, the aforementioned alkyl group, alkenyl group, and alkynyl group deuterated, and a substituted or unsubstituted alicyclic group having 3 to 6 carbon atoms.

Specific examples of the linear or branched alkyl group having 1 to 6 carbon atoms represented by R³³ can include a methyl group, an ethyl group, a propyl group, an isopropyl group, a n-butyl group, an isobutyl group, a sec-butyl group, a tert-butyl group, a n-pentyl group, a 1-methylbutyl group, a 2-methylbutyl group, a 3-methylbutyl group, a 1,1-dimethylpropyl group, a 1,2-dimethylpropyl group, a 2,2-dimethylpropyl group, a 3-pentyl group, and a n-hexyl group.

Specific examples of the linear or branched alkenyl group having 2 to 6 carbon atoms represented by R³³ can include a vinyl group, a 1-propenyl group, an allyl group, an isopropenyl group, a 1-butenyl group, a 2-butenyl group, a 3-butenyl group, a 1,3-butanedienyl group, a 1-ethylvinyl group, a 1-methyl-1-propenyl group, a 2-methyl-1-propenyl group, a 2-methyl-2-propenyl group, a pentenyl group, a pentadienyl group, a hexenyl group, a hexadienyl group, and a hexatrienyl group.

Specific examples of the linear or branched alkynyl group having 2 to 6 carbon atoms represented by R³³ can include an ethynyl group, a 1-propynyl group, a 2-propynyl group, a 1-butynyl group, a 2-butynyl group, a 3-butynyl group, a 1,3-butanediynyl group, a 1-methyl-2-propynyl group, a pentynyl group, a pentadiynyl group, a hexynyl group, a hexadiynyl group, and a hexatriynyl group.

The linear or branched alkyl group having 1 to 6 carbon atoms, the linear or branched alkenyl group having 2 to 6 carbon atoms, and the linear or branched alkynyl group having 2 to 6 carbon atoms may each be substituted, and examples of the substituent can include halogen (e.g., fluorine, chlorine, bromine, and iodine), a hydroxyl group, a linear or branched alkyloxy group having 1 to 4 carbon atoms, a linear or branched alkenyloxy group having 2 to 4 carbon atoms, a linear or branched alkynyloxy group having 2 to 4 carbon atoms, a cyano group, and a linear or branched sulfonyl group having 2 to 4 carbon atoms.

Examples of the substituted or unsubstituted linear or branched deuterated alkyl group having 1 to 6 carbon atoms, the substituted or unsubstituted linear or branched deuterated alkenyl group having 2 to 6 carbon atoms, and the substituted or unsubstituted linear or branched deuterated alkynyl group having 2 to 6 carbon atoms represented by R³³ can include the aforementioned substituted or unsubstituted linear or branched alkyl group having 1 to 6 carbon atoms, substituted or unsubstituted linear or branched alkenyl group having 2 to 6 carbon atoms, and substituted or unsubstituted linear or branched alkynyl group having 2 to 6 carbon atoms, in which one or more hydrogen atoms on carbon are substituted by deuterium.

Specific examples of the alicyclic group having 3 to 6 carbon atoms represented by R³³ can include a cyclopropyl group, a cyclopropenyl group, a cyclobutyl group, a cyclobutenyl group, a cyclobutadienyl group, a cyclopentyl group, a cyclopentenyl group, a cyclopentadienyl group, a cyclohexyl group, a cyclohexenyl group, and a cyclohexadienyl group.

The alicyclic group may be substituted, and examples of the substituent can include halogen (e.g., fluorine, chlorine, bromine, and iodine), a hydroxyl group, a linear or branched alkyloxy group having 1 to 4 carbon atoms, a linear or branched alkenyloxy group having 2 to 4 carbon atoms, a linear or branched alkynyloxy group having 2 to 4 carbon atoms, a cyano group, and a linear or branched sulfonyl group having 1 to 4 carbon atoms.

R³¹ mentioned above can be any of the groups listed above and is preferably a methyl group, an ethyl group, a vinyl group, an ethynyl group, an isopropyl group, an isopropenyl group, a n-butyl group, a sec-butyl group, an isobutyl group, a tert-butyl group, a hepta-deuterated isopropyl group, a 1,1,1,3,3,3-hexa-deuterated isopropyl group, a cyclohexyl group, a cyclopentyl group, a cyclohexyl group, a 2-methoxy-2-propyl group, a 2-methanesulfonyl-2-propyl group, a 2-methoxy-2-butyl group, or a 2-methanesulfonyl-2-butyl group, more preferably an isopropyl group, a sec-butyl group, a tert-butyl group, a hepta-deuterated isopropyl group, a 1,1,1,3,3,3-hexa-deuterated isopropyl group, a 2-methoxy-2-propyl group, or a 2-methanesulfonyl-2-propyl group, most preferably an isopropyl group.

R³⁴ in the formula (III) is hydrogen, a hydroxymethyl group or a hydroxymethyl group in which hydrogen on carbon is substituted by deuterium. Of them, hydrogen is preferred as R³⁴.

R³⁵ in the formula (III) is any one group selected from hydrogen, a hydroxyl group, a substituted or unsubstituted nitrogen-containing heterocyclic group having a 5- or 6-membered ring, and a substituted or unsubstituted nitrogen-containing aromatic group having 2 to 10 carbon atoms.

Specific examples of the nitrogen-containing heterocyclic group having a 5- or 6-membered ring represented by R³⁵ can include a pyrrolidinyl group, a pyrazolidinyl group, an imidazolidinyl group, an isoxazolidinyl group, an oxazolidinyl group, an isothiazolidinyl group, a thiazolidinyl group, a piperidinyl group, a hexahydropyridazinyl group, a hexahydropyrimidinyl group, a piperazinyl group, a hexahydrotriazinyl group, an oxazinanyl group, a thiazinanyl group, a morpholino group, and a thiomorpholino group.

The nitrogen-containing heterocyclic group having a 5- or 6-membered ring may be carbonylated at 1 or 2 carbon atoms in the ring and may be oxidized at one or more nitrogen atoms or sulfur atoms contained in the heterocyclic ring. The carbonylation refers to the replacement of CH₂ with C═O. In the case of oxidizing sulfur atoms, the sulfur atoms can be oxidized with 1 or 2 oxygen atoms. The nitrogen-containing heterocyclic group may be ring-fused with 1 or 2 benzene rings. The nitrogen-containing heterocyclic group may be substituted, and examples of the substituent can include halogen (e.g., fluorine, chlorine, bromine, and iodine), a hydroxyl group, a linear or branched alkyl group having 1 to 4 carbon atoms, a linear or branched alkenyl group having 2 to 4 carbon atoms, a linear or branched alkynyl group having 2 to 4 carbon atoms, an acetyl group, and a linear or branched sulfonyl group having 1 to 4 carbon atoms.

Specific examples of the nitrogen-containing aromatic group having 2 to 10 carbon atoms represented by R³⁵ can include a pyrrolyl group, a pyrazolyl group, an imidazolyl group, an isoxazolyl group, an oxazolyl group, an oxadiazolyl group, an isothiazolyl group, a thiazolyl group, a thiadiazolyl group, a pyridinyl group, a pyridazinyl group, a pyrimidinyl group, a pyrazinyl group, a triazinyl group, an indolyl group, an isoindolyl group, a benzoxazolyl group, a benzothiazolyl group, a benzisoxazolyl group, a benzisothiazolyl group, a benzoxadiazolyl group, a benzothiadiazolyl group, a pyrrolopyridinyl group, a pyrrolopyrazinyl group, a purinyl group, a quinolinyl group, an isoquinolinyl group, a cinnolinyl group, a quinazolinyl group, and a quinoxalinyl group.

The nitrogen-containing aromatic group having 2 to 10 carbon atoms may be substituted, and examples of the substituent can include halogen (e.g., fluorine, chlorine, bromine, and iodine), a hydroxyl group, a linear or branched alkyl group having 1 to 4 carbon atoms, a linear or branched alkenyl group having 2 to 4 carbon atoms, a linear or branched alkynyl group having 2 to 4 carbon atoms, an acetyl group, and a linear or branched sulfonyl group having 1 to 4 carbon atoms.

R³⁵ mentioned above can be any of the groups listed above and is preferably hydrogen, a hydroxyl group, a pyrrolidinyl group, a hydroxypyrrolidinyl group, a fluoropyrrolidinyl group, a difluoropyrrolidinyl group, an oxopyrrolidinyl group, an oxazolidinyl group, an oxooxazolidinyl group, an isothiazolyl group, a dioxoisothiazolyl group, a pyrazolidinyl group, a methylpyrazolidinyl group, a pyrazolidinyl group, a methyloxopyrazolidinyl group, a piperidinyl group, an oxopiperidinyl group, a piperazinyl group, a methylpiperazinyl group, an acetylpiperazinyl group, a morpholino group, a thiomorpholino group, a pyridinyl group, an oxadiazolyl group, a methyloxadiazolyl group, or a thiazolyl group, more preferably hydrogen, a hydroxyl group, a hydroxypyrrolidinyl group, a fluoropyrrolidinyl group, a difluoropyrrolidinyl group, an oxopyrrolidinyl group, an oxooxazolidinyl group, a dioxoisothiazolyl group, a pyrazolidinyl group, a methylpyrazolidinyl group, a pyrazolidinyl group, a methyloxopyrazolidinyl group, an oxopiperidinyl group, a methylpiperazinyl group, an acetylpiperazinyl group, a morpholino group, a thiomorpholino group, a pyridinyl group, a methyloxadiazolyl group, or a thiazolyl group, most preferably hydrogen.

R³⁶ in the formula (III) is any one group selected from hydrogen, a hydroxyl group, and halogen. Examples of the halogen can include fluorine, chlorine, bromine, and iodine. The group preferred as R¹⁶ is hydrogen.

Y in the formula (III) is any one substituent selected from methylene, deuterated methylene, and hydroxymethylene. The deuterated methylene refers to methylene in which 1 or 2 hydrogen atoms are substituted by deuterium. The hydroxymethylene refers to methylene in which one hydrogen atom is substituted by a hydroxyl group. Y is most preferably methylene.

The compound represented by the formula (III) can be any compound as long as the compound satisfies the conditions mentioned above. Specific examples thereof can include the following compound (III-1):

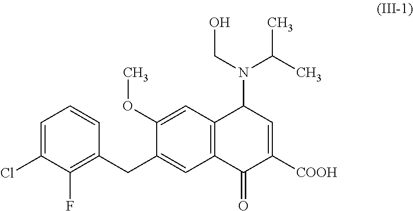 [SMILES: COc1cc2c(cc1Cc1cccc(Cl)c1F)C(=O)C(C(=O)O)=CC2N(CO)C(C)C]

When the compound selected from the formulas (I) to (IV) has an asymmetric carbon atom and an axial chirality-related asymmetric point, this compound includes all possible optical isomers. These optical isomers can be used at an arbitrary ratio. For example, a certain optically active compound can be used as an enantiomer, a racemate, or an enantiomer mixture at an arbitrary ratio. A compound containing a plurality of asymmetric points may be used as a diastereomer mixture at an arbitrary ratio.

When the compound selected from the formulas (I) to (IV) has a double bond, this compound includes all possible structural isomers. These structural isomers can be used as a mixture at an arbitrary ratio.

Examples of the pharmacologically acceptable salt of the compound of any of the formulas (I) to (IV) can include an acid-addition salt, a metal salt, an ammonium salt, and an organic amine-addition salt. Examples of the acid-addition salt can include: a salt with each inorganic acid such as hydrochloric acid, hydrobromic acid, sulfuric acid, nitric acid, phosphoric acid, and boric acid; and an organic acid such as a carboxylic acid, a sulfonic acid, and an amino acid. Examples of the metal salt can include: a salt with each alkali metal such as lithium, sodium, and potassium; a salt with each alkaline earth metal such as magnesium and calcium; and a salt with each metal such as aluminum and zinc. Examples of the ammonium salt can include a salt with ammonium, tetramethylammonium, and the like. Examples of the organic amine salt can include a salt with triethylamine, piperidine, morpholine, toluidine, and the like.

Examples of the carboxylic acid can particularly include a substituted or unsubstituted alkylcarboxylic acid having 1 to 6 carbon atoms, and a substituted or unsubstituted dicarboxylic acid having 4 to 8 carbon atoms. Specific examples of the substituted or unsubstituted alkylcarboxylic acid having 1 to 6 carbon atoms can include formic acid, acetic acid, propionic acid, isopropionic acid, butyric acid, isobutyric acid, valeric acid, isovaleric acid, caproic acid, lactic acid, glycolic acid, levulinic acid, and oxahexanoic acid. The alkylcarboxylic acid may be substituted, and examples of the substituent can include halogen (e.g., fluorine, chlorine, bromine, and iodine), a cyano group, a nitro group, and an aromatic group. In particular, aromatic group-substituted propionic acid is preferably used. Examples of the substituted or unsubstituted dicarboxylic acid having 4 to 8 carbon atoms can include succinic acid, maleic acid, fumaric acid, maleic acid, glutaric acid, oxoglutaric acid, adipic acid, oxoadipic acid, pimelic acid, suberic acid, cyclohexanedicarboxylic acid, oxalic acid, phthalic acid, and terephthalic acid. The dicarboxylic acid may be substituted, and examples of the substituent can include halogen (e.g., fluorine, chlorine, bromine, and iodine), a cyano group, and a nitro group. The carboxylic acid is most preferably acetic acid, levulinic acid, lactic acid, flurbiprofen, ketoprofen, oxalic acid, fumaric acid, or maleic acid.

Examples of the sulfonic acid particularly include sulfonic acid having 1 to 10 carbon atoms and specifically include methanesulfonic acid, trifluoromethanesulfonic acid, ethanesulfonic acid, butanesulfonic acid, benzenesulfonic acid, toluenesulfonic acid, mesitylenesulfonic acid, naphthalenesulfonic acid, and camphorsulfonic acid. The sulfonic acid is preferably methanesulfonic acid, trifluoromethanesulfonic acid, or ethanesulfonic acid, most preferably methanesulfonic acid.

When the organic amine, the carboxylic acid, the sulfonic acid, or the amino acid has an asymmetric carbon atom, this compound includes all possible optical isomers. These optical isomers can be used at an arbitrary ratio. For example, a certain optically active compound can be used as an enantiomer, a racemate, or an enantiomer mixture at an arbitrary ratio. A compound containing a plurality of asymmetric points may be used as a diastereomer mixture at an arbitrary ratio.

Exemplary methods for producing the compounds (I) to (IV) used in the therapeutic agent for type 2 diabetes of the present invention will be described below. However, the present invention is not intended to be limited by these production methods. Also, some compounds can be obtained as commercially available products.

The compound represented by any of the formulas (I) and (IV) can be synthesized by use of Mannich reaction. As shown in the reaction scheme given below, an acetophenone derivative, a ketone or an aldehyde, and a piperidine derivative can be reacted under Mannich reaction conditions to obtain a Mannich condensate. In this reaction, proline or the like can also be used as a catalyst to enantio-selectively synthesize the compound represented by any of the formulas (I) and (IV).

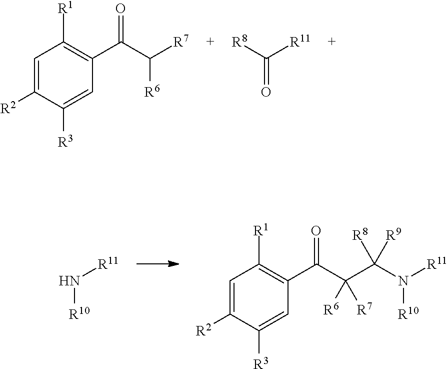

R⁴ is introduced to carbonyl in the obtained Mannich condensate by the action of a nucleophile, while the resulting hydroxyl group can be mesylated or converted to a leaving group such as halogen, followed by the introduction of R⁵ through S_{N}2 reaction to obtain the compound represented by any of the formulas (I) and (IV).

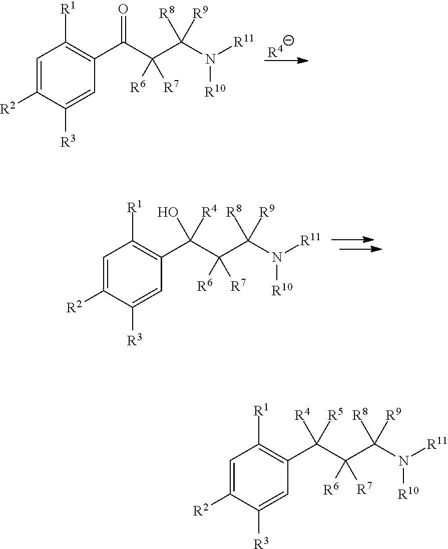

Specifically, as shown in the reaction scheme given below, 4-ethyl phenyl ethyl ketone, formaldehyde, and piperidine can be used as starting materials to synthesize a compound represented by the formula (I-1).

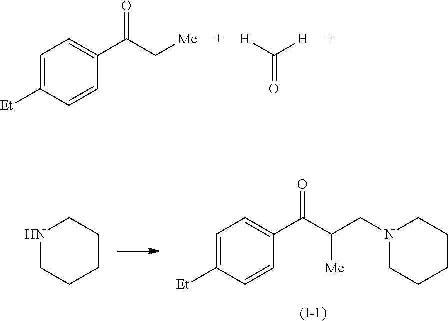

For the compound represented by the formula (I) wherein R⁸ and R⁹ form a carbonyl group together with carbon bonded thereto, amine or amide having R¹⁰ and R¹¹ is formed with a cinnamic acid derivative as a starting material, and R⁵ can then be introduced thereto through Michael reaction, followed by the introduction of R⁷ through electrophilic reaction with enol ether as a nucleophile.

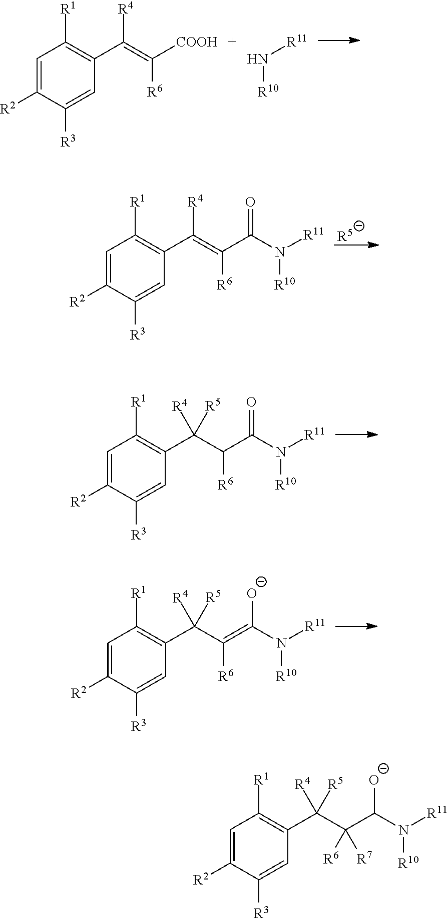

The compound represented by the formula (II) can be synthesized, for example, using 3-aminopropanol having a protected amino group as a starting material. In this context, for example, an amide carbonate protective group such as a tert-butoxycarbonyl (Boc) group or a benzyloxycarbonyl (Cbz) group, a benzyl protective group such as a benzyl (Bz) group or a 4-methoxybenzyl (PMB) group, or phthalimide can be used as the protective group (PG). When R²¹ is hydrogen, 3-aminopropanol having a protected amino group can be subjected to Mitsunobu reaction mentioned later. When R²¹ is not hydrogen, the alcohol moiety of 3-aminopropanol having a protected amino group is oxidized into aldehyde, and R²¹ is then introduced thereto. Any condition can be used as the oxidation conditions. For example, oxidation reaction using dimethyl sulfoxide, such as Swern oxidation or SO₃ pyridine oxidation, oxidation reaction using hypervalent iodine such as iodosobenzene diacetate or Dess-Martin periodinane, or TEMPO oxidation can be used. The obtained aldehyde can be nucleophilically reacted with R²¹ to synthesize a secondary alcohol carrying R²¹. Examples of the specific reaction can include a method which involves subjecting a R²¹-halogen compound to halogen-lithium exchange using an organic lithium reagent to form a R¹-anion, which is then reacted with an aldehyde, and a method which involves converting a R²¹-halogen compound to a Grignard reagent, which is then reacted with an aldehyde. The target secondary alcohol carrying R²¹ can also be synthesized by use of Nozaki-Hiyama-Kishi (NHK) reaction. In particular, for the enantio-selective synthesis of the secondary alcohol, asymmetric NHK reaction is preferably used. The secondary alcohol thus obtained or the aforementioned 3-aminopropanol having a protected amino group and a phenol derivative can be coupled through Mitsunobu reaction to obtain the backbone of the compound represented by the formula (II). Thereafter, the protective group of the amino group is removed by an appropriate method, for example, under acidic conditions for the Boc group and the Cbz group, through hydrogenation reaction using zerovalent palladium as a catalyst for the Bz group and the 4-methoxybenzyl PMB group, or by the addition of hydrazine for the phthalimide, and R²² can then be introduced onto the amino group to synthesize the compound represented by the formula (II).

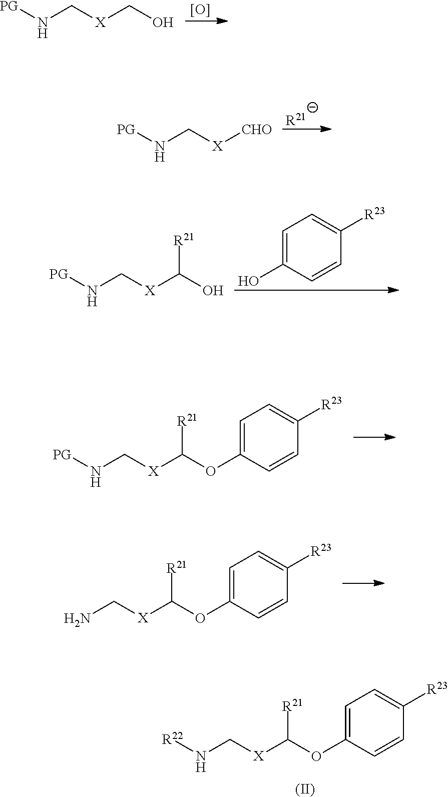

Specifically, 3-phenyl-3-(4-trifluoromethylphenyl)-N-methylpropylamine represented by the formula (II-1) can be synthesized with N-tert-butoxycarbonyl-3-aminopropanol as a starting material as shown in the following reaction scheme:

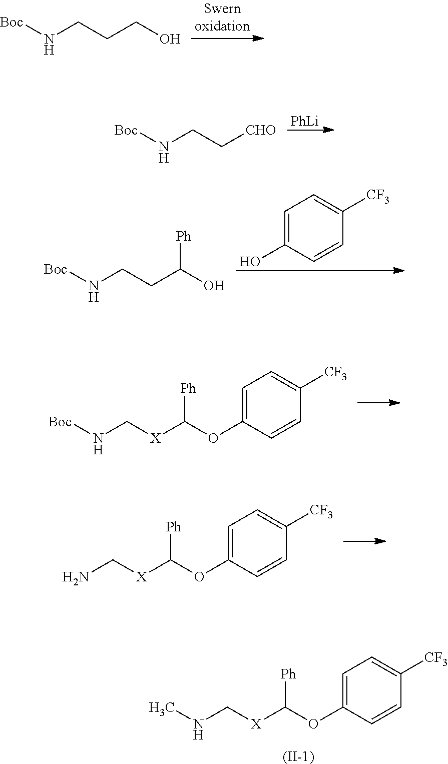

The compound represented by the formula (II) can also be synthesized by use of Mannich reaction. The carbonyl compound obtained by the Mannich reaction is reduced into a secondary alcohol, and the compound represented by the formula (II) can then be obtained in the same way as above. Also, an enantiomer of the compound represented by the formula (II) can be obtained by use of reduction reaction and asymmetric reduction reaction such as Noyori reaction.

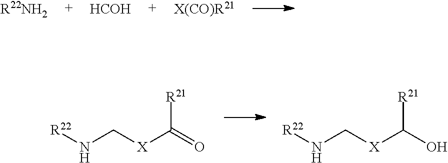

The compound represented by the formula (III) can be synthesized with reference to a method described in Japanese unexamined Patent Application Publication No. 2006-1927. For example, the compound represented by the formula (III) can be synthesized with acid chloride of 2,4-difluoro-5-iodobenzoic acid as a starting material. This acid chloride is reacted with, for example, 2-N,N-dialkylaminoacrylic acid ester such as ethyl 2-N,N-dimethylaminoacrylate, under basic conditions, and primary amine is subsequently added thereto for the exchange reaction of the amine. Subsequently, the 4-quinoline skeleton of the right segment of the compound represented by the formula (III) is constructed by the action of a base. This right segment and the left segment are then linked by Negishi coupling to construct the backbone of the compound represented by the formula (III). In this respect, a zinc reagent derived from the left segment can be prepared from the corresponding benzyl bromide derivative and activated zinc such as Rieke zinc. Then, the carboxylic acid moiety can be hydrolyzed, followed by S_{N} aryl reaction with an alkoxide anion as a nucleophile to synthesize the compound represented by the formula (III).

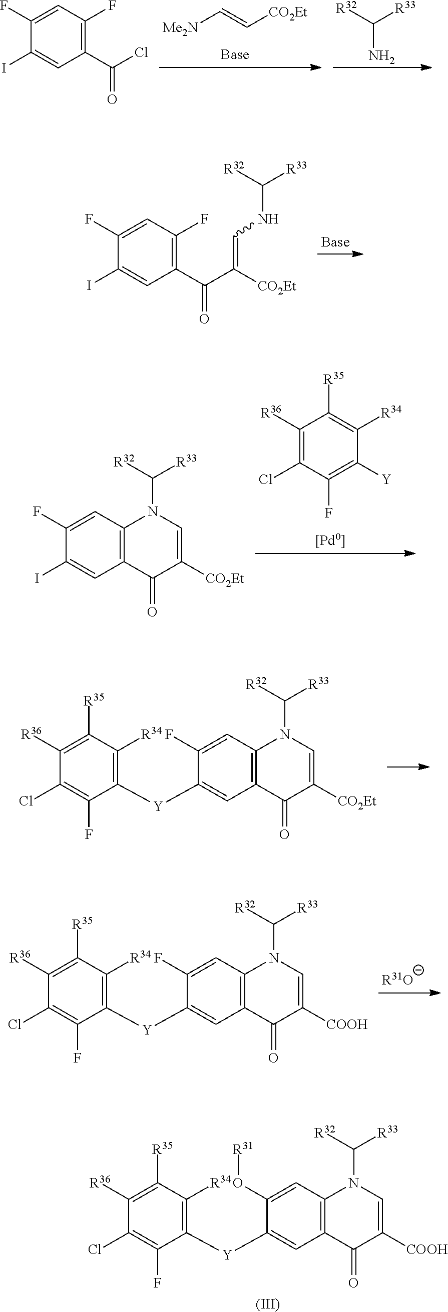

Specifically, a compound represented by the formula (III-1) can be synthesized with 2,4-difluoro-5-iodobenzoic acid chloride as a starting material as shown in the following reaction scheme:

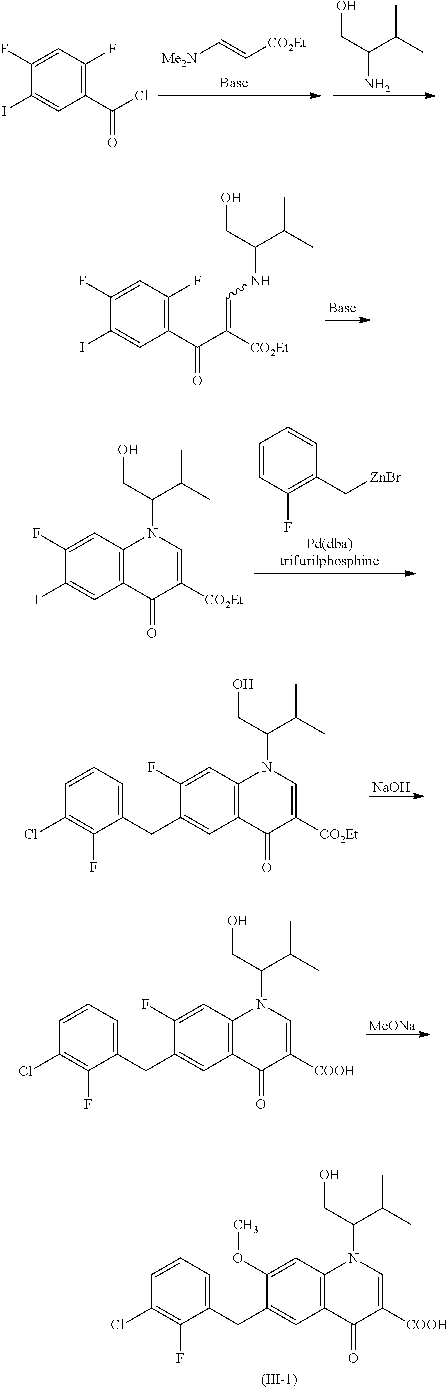

For the synthesis of the compound of the present invention, a protective group can be appropriately used according to the structure of the compound. Such a protective group can be used with reference to Green & Wuts, “PROTECTIVE GROUPS in ORGANIC SYNTHESIS” 3rd ed., John Wiley & Sons, Inc.

Some compounds represented by the formulas (I) to (III) can also be obtained as commercially available products.

The “Cdkal1 gene mutation” according to the present invention means that one or more nucleotides in DNA or RNA of the Cdkal1 gene are substituted by another base, one or more nucleotides are inserted to DNA or RNA of the Cdkal1 gene, or one or more nucleotides in DNA or RNA of the Cdkal1 gene are deleted. Such nucleotide substitution, insertion, or deletion may occur at a plurality of positions in the DNA or RNA of the Cdkal1 gene, and different mutations may occur at the same time.

The therapeutic agent for type 2 diabetes, particularly, the therapeutic agent for type 2 diabetes with Cdkal1 gene mutation resulting in the reduced ability to secrete insulin, the treatment kit for this disease, the agent activating the conversion of proinsulin to insulin, or the activation kit for the conversion of proinsulin to insulin according to the present invention can be further used in combination with one or more therapeutic agents for diabetes selected from various sulfonylurea drugs, various phenylalanine derivatives, various biguanide drugs, various α-glucosidase inhibitors, various thiazoline derivatives, various GLP-1 receptor agonists, and the like. The therapeutic agent for type 2 diabetes, the treatment kit for this disease, the agent activating the conversion of proinsulin to insulin, or the activation kit for the conversion of proinsulin to insulin according to the present invention differs from these existing therapeutic agents for diabetes in the mechanism of action and as such, can be expected to produce additive, and in some cases, synergistic effects when used in combination with the therapeutic agent(s) for type 2 diabetes.

Examples of the administration route of each ingredient in the therapeutic agent for type 2 diabetes, particularly, the therapeutic agent for type 2 diabetes with Cdkal1 gene mutation resulting in the reduced ability to secrete insulin, or the treatment kit for this disease according to the present invention, each ingredient in the combination of therapeutic agents, or each ingredient in the agent activating the conversion of proinsulin to insulin or the activation kit for the conversion of proinsulin to insulin according to the present invention can include: oral administration also including sublingual administration; and parenteral administration such as intravenous administration including nasal dripping, inhalation, and drip infusion, percutaneous administration through cataplasms or the like, and administration by use of suppositories or a forced enteral nutrition method using a nasogastric tube, a nasoenteric tube, a gastrostomy tube, or an enteral tube. An administration route already approved for each agent is preferably adopted as the administration route of the therapeutic agent for type 2 diabetes in the combination of therapeutic agents.

The dosage form of each ingredient in the therapeutic agent for type 2 diabetes, particularly, the therapeutic agent for type 2 diabetes with Cdkal1 gene mutation resulting in the reduced ability to secrete insulin, or the treatment kit for this disease according to the present invention, or the dosage form of each ingredient in the agent activating the conversion of proinsulin to insulin or the activation kit for the conversion of proinsulin to insulin according to the present invention can be appropriately determined according to the administration route mentioned above. Examples thereof can include an injection, nasal drops, drops, a tablet, a capsule, fine granules, a powder, a solution, a liquid agent prepared by dissolution in a syrup or the like, a cataplasm, and a suppository. Each ingredient in the therapeutic agent for type 2 diabetes or the treatment kit for this disease according to the present invention, or each ingredient in the agent activating the conversion of proinsulin to insulin or the activation kit for the conversion of proinsulin to insulin according to the present invention can be used for medical purposes as well as in a supplement form of a tablet or a capsule. In particular, for the elderly, etc., having difficulty swallowing, the form of a disintegrating tablet that exhibits rapidly disintegrating properties in the mouth or the form of a solution suitable for nasogastric administration is preferred.

In order to prepare the therapeutic agent for type 2 diabetes, particularly, the therapeutic agent for type 2 diabetes with Cdkal1 gene mutation resulting in the reduced ability to secrete insulin, the treatment kit for this disease, the agent activating the conversion of proinsulin to insulin, or the activation kit for the conversion of proinsulin to insulin according to the present invention, a pharmacologically acceptable carrier, excipient, diluent, additive, disintegrant, binder, coating agent, lubricant, glidant, lubricating agent, flavor, sweetener, solubilizing agent, solvent, gelling agent, nutrient, and the like can be added, if necessary. Specific examples thereof can include water, saline, animal-derived fat and oil, plant oil, lactose, starch, gelatin, crystalline cellulose, gum, talc, magnesium stearate, hydroxypropylcellulose, polyalkylene glycol, polyvinyl alcohol, and glycerin.

The therapeutic agent for type 2 diabetes, particularly, the therapeutic agent for type 2 diabetes with Cdkal1 gene mutation resulting in the reduced ability to secrete insulin, the treatment kit for this disease, the agent activating the conversion of proinsulin to insulin, or the activation kit for the conversion of proinsulin to insulin according to the present invention can be used for humans as well as for livestock, fowls, pets, and the like in the veterinary field. The dose, frequency, and period of the administration of such a therapeutic agent, etc., for a human subject differs depending on the age, body weight, symptoms, etc., of the type 2 diabetes patient. Examples of the dose of the compound represented by any of the formulas (I) to (III) or the pharmaceutically acceptable salt thereof can include 0.01 mmol to 25 mmol/day, preferably 0.025 mmol to 7.5 mmol/day, more preferably 0.075 mmol to 5.5 mmol/day, further preferably 0.2 mmol to 2 mmol/day, particularly, 0.45 mmol to 1.3 mmol/day, per adult in terms of each compound. Examples of the frequency of administration can include administration once or more a day and continuous administration using drip infusion or the like. The administration period can also be determined according to a method known to a pharmacologist or a clinician in the art. In this case, the administration period can also be determined with a blood glucose level or a serum insulin level as an index.

Hereinafter, the present invention will be described more specifically with reference to Examples. However, the technical scope of the present invention is not intended to be limited by these examples.

### [Synthesis of Compound]

Each compound described herein can be prepared by a method known in the art, or a commercially available product can also be used. The compounds (I-1) to (I-61), (II-1), (II-2), and (III-1) were purchased from Namiki Shoji Co., Ltd. (Tokyo, Japan).

Hereinafter, synthesis examples of the compounds (IV-1) to (IV-4), which are novel compounds, will be shown.

### Production Example 1

### Synthesis of Compound (IV-1)

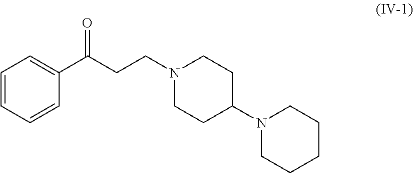 [SMILES: O=C(CCN1CCC(N2CCCCC2)CC1)c1ccccc1]

To 3-chloro-1-phenyl-1-propanone (253 mg, 1.5 mmol) in acetone (20 mL), anhydrous potassium carbonate (414 mg, 3 mmol) was added, and the mixture was stirred at room temperature for 1 hour. To the reaction solution, a solution of 4-piperidinopiperidine (1.5 mmol) in acetone (10 mL) was then added, and the mixture was reacted for 20 hours by heating to 45 to 47° C. The completion of the reaction was confirmed by thin-layer chromatography, and the solvent was then distilled off under reduced pressure. To the residue, water (20 ml) was added. The aqueous layer was subjected to extraction with ethyl acetate (50 mL) three times. The organic layer was washed with water and saline and then dried over anhydrous magnesium sulfate. After filtration, the solvent was distilled off under reduced pressure to obtain the title compound as a pale yellow solid at a yield of 77%.

¹H NMR (300 MHz, CDCl₃) δ 2.24-2.33 (m, 5H), 2.50-2.61 (m, 10H), 2.82-2.92 (m, 2H), 3.16-3.26 (m, 2H), 7.44-7.62 (m, 3H), 7.94-8.03 (m, 2H)

MS (TOF Mass): m/z calcd for C₁₉H₂₈N₂O (M+1) 301.22. found: 301.23.

### Production Example 2

### Synthesis of Compound (IV-2)

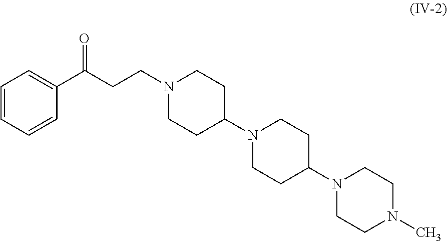 [SMILES: CN1CCN(C2CCN(C3CCN(CCC(=O)c4ccccc4)CC3)CC2)CC1]

Compound (IV-2) was obtained as a white solid at a yield of 79% in the same way as in Production Example 1 except that 1-methyl-4-[1-(4-piperidyl)-4-piperidyl]piperazine was used instead of 4-piperidinopiperidine.

¹H NMR (300 MHz, CDCl₃) δ 1.59-1.63 (m, 5H), 1.76-1.86 (m, 4H), 2.03 (t, J=12 Hz, 2H), 2.27 (s, 3H), 2.47-2.62 (m, 11H), 2.80 (t, J=12 Hz, 3H), 2.90 (t, 15 Hz, 3H), 3.19 (t, J=13 Hz, 2H), 7.47 (t, J=8.0 Hz, 2H), 7.57 (t, J=7.2 Hz, 1H), 7.96 (d, J=7.3 Hz, 2H)

MS (TOF Mass): m/z calcd for C₂₄H₃₈N₄O (M+1) 399.31. found: 399.31.

### Production Example 3

### Synthesis of Compound (IV-3)

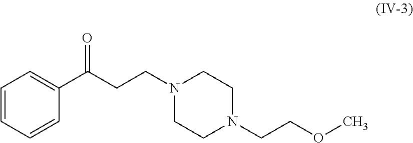 [SMILES: COCCN1CCN(CCC(=O)c2ccccc2)CC1]

Compound (IV-3) was obtained as a yellow oil at a yield of 86% in the same way as in Production Example 1 except that 1-(2-methoxyethyl)piperazine was used instead of 4-piperidinopiperidine.

¹H NMR (300 MHz, CDCl₃) δ 2.12-2.15 (m, 10H), 2.81-2.83 (m, 2H), 3.30 (s, 3H), 3.45-3.50 (m, 2H), 7.31-7.48 (m, 2H)

MS (TOF Mass): m/z calcd for C₁₆H₂₄N₂O₂ (M+1) 277.19. found: 277.18.

### Production Example 4

### Synthesis of Compound (IV-4)

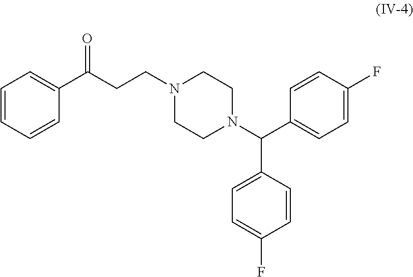 [SMILES: O=C(CCN1CCN(C(c2ccc(F)cc2)c2ccc(F)cc2)CC1)c1ccccc1]

Compound (IV-4) was obtained as a white solid at a yield of 73% in the same way as in Production Example 1 except that 1-[bis(4-fluorophenyl)methyl]piperazine was used instead of 4-piperidinopiperidine.

¹H NMR (300 MHz, CDCl₃) δ 2.41-2.55 (m, 10H), 2.82-2.88 (m, 2H), 3.16-3.22 (m, 2H), 4.21 (s, 1H), 6.92-7.01 (m, 4H), 7.31-7.38 (m, 2H), 7.42-7.58 (m, 4H), 7.92-7.98 (m, 2H)

MS (TOF Mass): m/z calcd for C₂₆H₂₆F₂N₂O (M+1) 421.20. found: 421.23.

### Example 1

### Primary Screening

(1) Preparation of plasmid pACYCDuet-1-Phe

The vector map of the prepared plasmid pACYCDuet-1-Phe (SEQ ID NO: 1) is shown in the left diagram of FIG. 1. The phenylalanine codon TTT was designed as NNN. When translation takes place according to the normal reading frame, the stop codon TAA appears immediately after TTT so that the sequence is not translated into firefly luciferase. On the other hand, if mistranslation occurs in TTT so as to skip one T, the stop codon is circumvented so that the sequence is correctly translated into firefly luciferase. This means that the higher the accuracy of translation is, the lower the fluorescence intensity of firefly luciferase is.

Renilla luciferase gene is also cloned into this plasmid as a control.

A nucleotide sequence (SEQ ID NO: 2) encoding firefly luciferase, a nucleotide sequence (SEQ ID NO: 3) encoding Renilla luciferase, and a sequence (SEQ ID NO: 4) for detecting mistranslation were transferred to a plasmid pACYCDuet-1 (manufactured by Novagen/Merck KGaA) to prepare pACYCDuet-1-Phe (SEQ ID NO: 1). E. coli DH5α (manufactured by Takara Bio Inc.) was transformed with the obtained pACYCDuet-1-Phe and cultured at a large scale. Then, the plasmid was extracted and purified using Plasmid Maxi prep kit (manufactured by Qiagen N.V.).

The nucleotide sequence of the obtained pACYCDuet-1-Phe was confirmed using ABI Prism 310 genetic analysis (manufactured by Applied Biosystems, Inc.).

### (2) Dual Luciferase Assay

The E. coli harboring pACYCDuet-1-Phe was cultured overnight at 37° C. using 100 mL of a culture medium. After confirmation that OD₅₅₀ reached 0.4 to 1, 1 mL of the culture medium was dispensed, and each low-molecular compound was added thereto at a final concentration of 10 μM. After shake culture for 1 hour, the E. coli was recovered and lysed by suspension in 10 mM Tris-HCl (pH 7.4), 1 mM magnesium chloride, and 0.1 mg/mL lysozyme (manufactured by Wako Pure Chemical Industries, Ltd.).

The activity of the firefly luciferase and the Renilla luciferase was measured using Dual-Luciferase Reporter Assay System (manufactured by Promega Corp.). To 5 μL of the E. coli lysate, 50 μL of an assay solution for the firefly luciferase was added for the measurement. Then, 50 μL of an assay solution for the Renilla luciferase was added thereto for the measurement. An E. coli lysate supplemented only with DMSO was prepared as a control.

The luminescence intensity of the firefly luciferase was corrected against that of the Renilla luciferase by use of the equation given below. This series of experimental approaches is plotted in FIG. 1.

Relative luminescence intensity=Firefly luciferase/Renilla luciferase

Relative translation accuracy=Relative luminescence intensity (from the addition of only DMSO)/Relative luminescence intensity (in the presence of the compound)

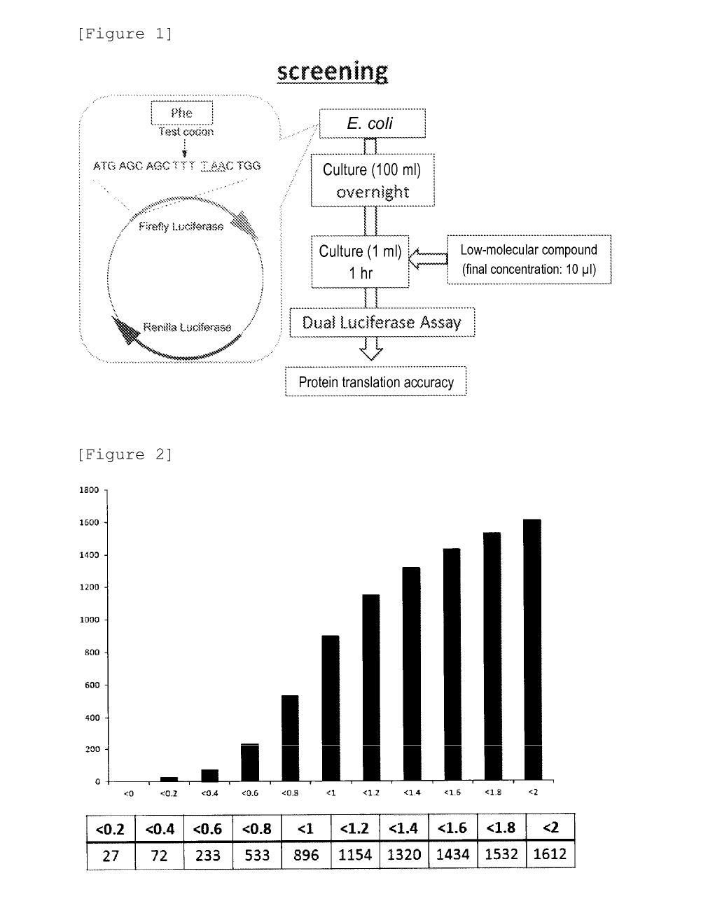

Results of studying the influence of each compound obtained by the primary screening on translation accuracy are shown in FIG. 2. The upper column of a table shown under the graph shows the value of relative luminescence intensity corrected according to the equation. In this context, a numeric value smaller than 1 means that translation accuracy was improved. The value in the lower column represents the cumulative number of compounds used in the primary screening.

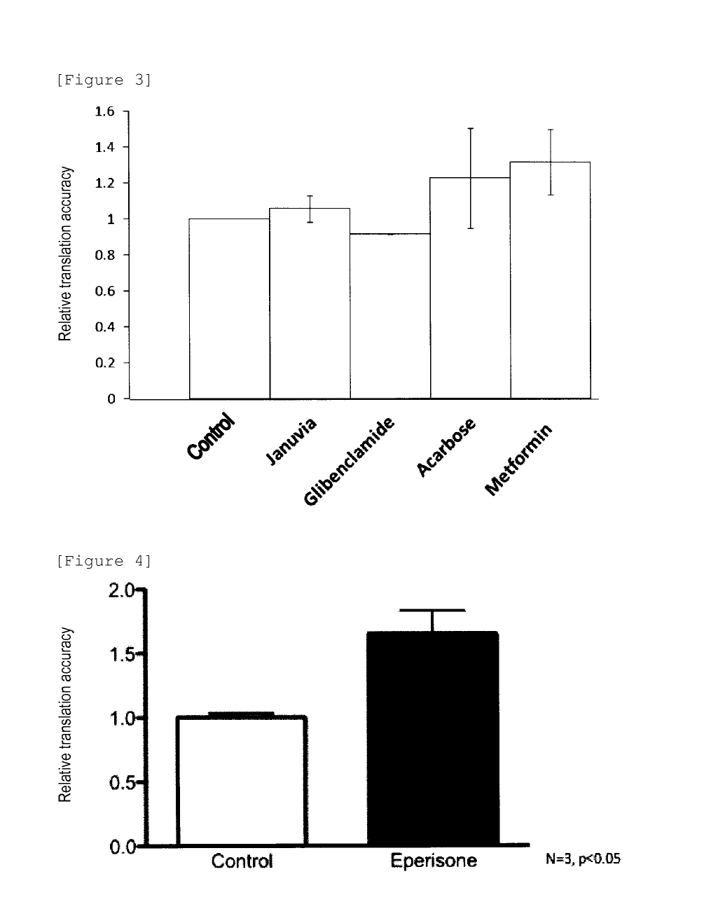

Results of screening existing therapeutic agents for diabetes, i.e., Januvia (manufactured by MSD K.K.), glibenclamide (manufactured by Wako Pure Chemical Industries, Ltd.), acarbose (manufactured by Sigma-Aldrich Corp.), and metformin (manufactured by Wako Pure Chemical Industries, Ltd.), by use of the screening method described above are shown in FIG. 3. The ordinate represents relative translation accuracy with the translation accuracy of the control defined as 1. As compared with the control, none of the existing therapeutic agents for diabetes were shown to significantly improve translation accuracy.

Results of screening eperisone (compound I-1) by use of the screening method describe above are shown in FIG. 4. The ordinate represents relative translation accuracy with the translation accuracy of the control defined as 1. As compared with the control, fluoxetine was shown to improve translation accuracy by approximately 1.5 times.

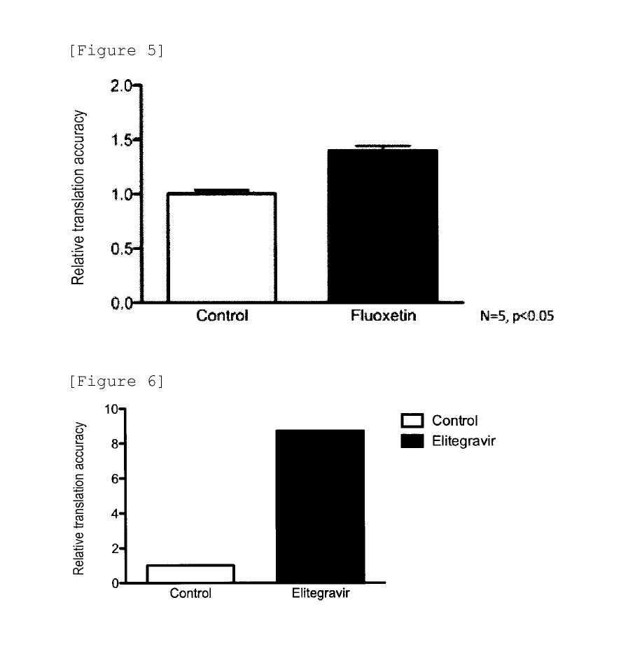

Results of screening fluoxetine (compound II-1) by use of the screening method describe above are shown in FIG. 5. The ordinate represents relative translation accuracy with the translation accuracy of the control defined as 1. As compared with the control, fluoxetine was shown to improve translation accuracy by approximately 1.5 times.

Results of screening elvitegravir (compound III-1) by use of the screening method describe above are shown in FIG. 6. The ordinate represents relative translation accuracy with the translation accuracy of the control defined as 1. As compared with the control, elvitegravir was shown to improve translation accuracy by approximately 8 times.

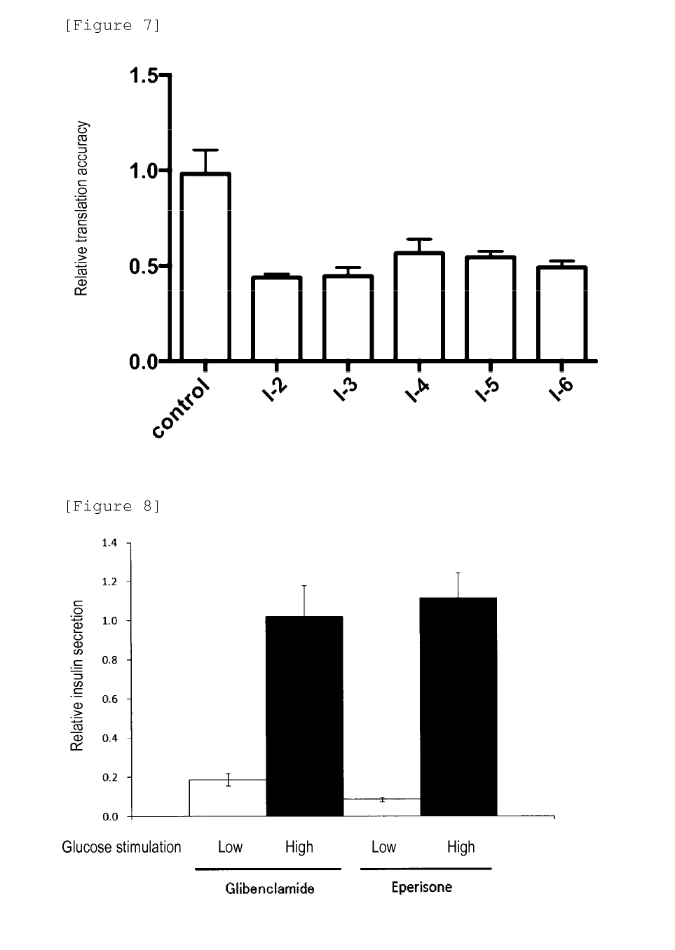

Results of screening compounds represented by the formulas I-2, I-3, I-4, I-5, and I-6, which are low-molecular compounds modified on the basis of a skeleton common to eperisone and fluoxetine, by use of the screening method describe above are shown in FIG. 7. The ordinate represents relative translation accuracy with the translation accuracy of the control defined as 1. As compared with the control, all of these 5 low-molecular compounds were shown to improve translation accuracy by 1.5 or more times.

### Example 2

### Secondary Screening

### (1) Preparation of Islet of Langerhans

The islet of Langerhans was isolated from a pancreatic β cell-specific Cdkal1-deficient mouse (see non-patent document 11) according to the literature (Gotoh M., et al., Transplantation, 1987, 43 (5), p. 725-730). The chest of the mouse was opened under ether anesthesia, and the common bile duct was peeled off. The pancreas swollen by the gradual injection of a collagenase solution (320 U/mL, manufactured by Sigma-Aldrich Corp.) was excised. The pancreas containing collagenase was digested for 30 minutes in a water bath of 37° C. and then dispersed with a pipette. The islet of Langerhans was isolated using a concentration gradient of a Ficoll solution (manufactured by Amersham Pharmacia Biotech Inc.). The islet of Langerhans was incubated for 30 minutes while kept at 37° C. in a Ringer solution (119 mM sodium chloride, 4.74 mM potassium chloride, 1.19 mM monosodium dihydrogen phosphate, 25 mM sodium bicarbonate, 10 mM HEPES, 2.54 mM calcium chloride, 1.19 mM magnesium chloride, and 0.2% BSA) saturated with a mixed gas of 95% O₂ and 5% CO₂.

### (2) Insulin Secretion Experiment by Glucose Stimulation

Each low-molecular compound found positive in the primary screening was dissolved in DMSO, and this solution was added at a final concentration of 10 mM to a Ringer solution containing glucose to prepare a Ringer solution. A Ringer solution supplemented only with DMSO was prepared as a control.

The isolated mouse islet of Langerhans was first cultured for 30 minutes in a Ringer solution containing a low concentration (2.8 mM) of glucose. Then, the Ringer solution was replaced with a Ringer solution containing the low-molecular compound and the low concentration of glucose. After 30 minutes, the Ringer solution was recovered, and a Ringer solution containing the low-molecular compound and a high concentration (16.7 mM) of glucose was subsequently added thereto. After 30 minutes, the Ringer solution was recovered. The amount of insulin released into the Ringer solution was detected using an insulin detection kit (Libs Insulin-Mouse (S type), manufactured by Shibayagi Co., Ltd) according to the protocol of the kit.

Results of screening glibenclamide, which is a therapeutic agent for type 2 diabetes classified as a sulfonylurea drug, and eperisone found positive in the primary screening, by the screening method described above are shown in FIG. 8. The ordinate represents the relative amount of insulin secreted with the amount of insulin secreted in the presence of glibenclamide under the high-concentration glucose stimulation defined as 1. Eperisone was shown to accelerate insulin secretion only under the high-concentration glucose stimulation. Eperisone was also shown to accelerate insulin secretion more highly than glibenclamide under the high-concentration glucose stimulation.

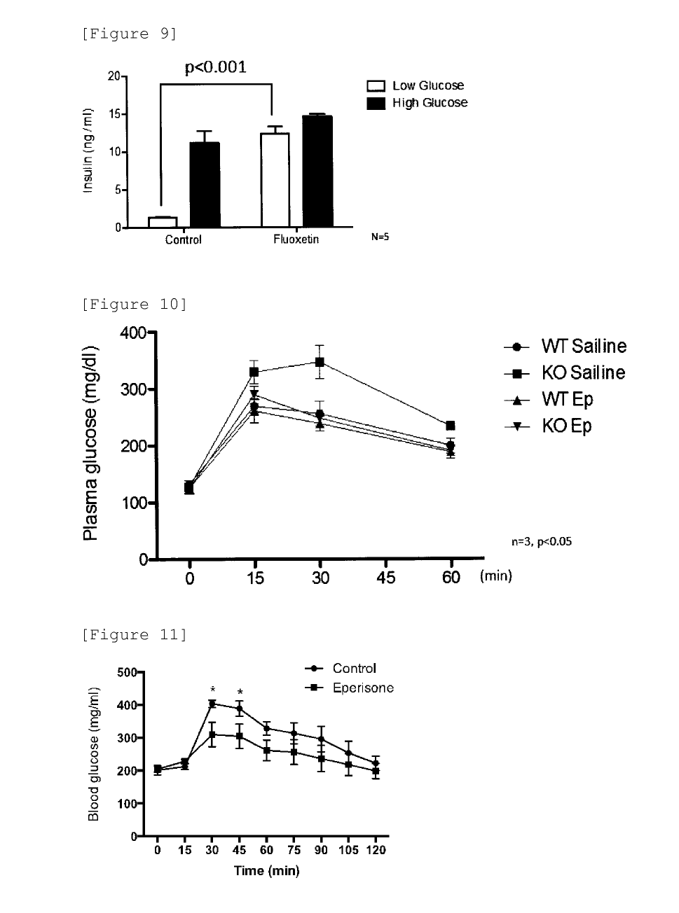

Results of screening fluoxetine by the screening method described above are shown in FIG. 9. The ordinate represents the concentration of insulin secreted into the Ringer solution. Fluoxetine was shown to significantly improve insulin secretion under the low-concentration glucose stimulation.

### Example 3

### Tertiary Screening

A pancreatic β cell-specific Cdkal1-deficient mouse and a wild-type mouse were each fasted overnight. Then, eperisone was transabdominally injected at a dose of 1 mg/kg to each mouse. Each control individual was prepared by the transabdominal injection of saline to each of a pancreatic β cell-specific Cdkal1-deficient mouse and a wild-type mouse. After 30 minutes, glucose was transabdominally injected at a dose of 1 g/kg to all of these mice. Blood was collected (5 μL) every 15 minutes from immediately after the administration of glucose. The blood glucose level in the mouse blood was measured using Accu-Chek AVIVA Nano (manufactured by F. Hoffmann-La Roche, Ltd.). The statistical test was conducted by repeated measure of two-way ANOVA.

Results of screening eperisone by use of the screening method described above are shown in FIG. 10. The ordinate represents the glucose concentration in blood. The abscissa represents the time elapsed after the administration of glucose. The pancreatic β cell-specific Cdkal1-deficient mouse that received eperisone was shown to have a significantly decreased blood glucose level as compared with the saline administration group.

### Example 4

### Improvement in Glucose Tolerance of Pancreatic β Cell-Specific Cdkal1-Deficient Mouse by Long-Term Administration of Low-Molecular Compound Found Positive in Screening

Each low-molecular compound found positive in the screening was transabdominally injected once a day over 14 days at a dose of 1 mg/kg to a pancreatic β cell-specific Cdkal1-deficient mouse. A Cdkal1-deficient mouse was prepared as a control by the injection of saline containing DMSO. Finally, glucose was transabdominally injected at a dose of 1 g/kg to all of the mice 36 hours after the administration of the low-molecular compound. Blood was collected (5 μL) from the tail vein every 15 minutes from immediately after the administration of glucose. The blood glucose level in the mouse blood was measured using Accu-Chek AVIVA Nano (manufactured by F. Hoffmann-La Roche, Ltd.). The statistical test was conducted by repeated measure of two-way ANOVA.

Results of screening eperisone by use of the screening method described above are shown in FIG. 11. The ordinate represents the glucose concentration in blood. The abscissa represents the time elapsed after the administration of glucose. The pancreatic β cell-specific Cdkal1-deficient mouse that received eperisone was shown to have a significantly decreased blood glucose level as compared with the DMSO administration group.

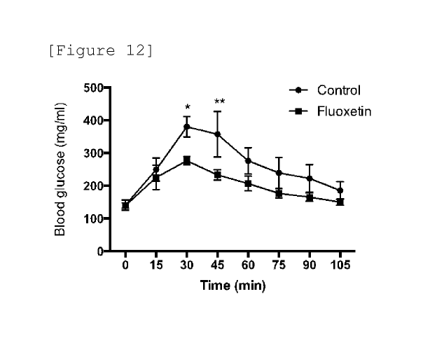

Results of screening fluoxetine by use of the screening method described above are shown in FIG. 12. The ordinate represents the glucose concentration in blood. The abscissa represents the time elapsed after the administration of glucose. The pancreatic β cell-specific Cdkal1-deficient mouse that received fluoxetine was shown to have a significantly decreased blood glucose level as compared with the DMSO administration group.

### INDUSTRIAL APPLICABILITY

The present invention can provide a novel therapeutic agent for a patient with type 2 diabetes with Cdkal1 gene mutation in pancreatic β cells resulting in the reduced ability to secrete insulin. This is useful in the medical world and the pharmaceutical industry.

1. A method for treating type 2 diabetes comprising administering to a subject one or more compounds selected from the group consisting of compounds represented by the following formula (I): 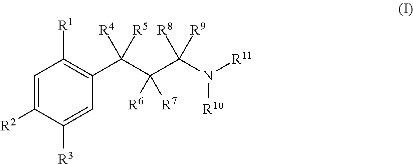
[wherein
R¹ represents any one group selected from hydrogen, a substituted or unsubstituted linear or branched alkyl group having 1 to 4 carbon atoms, a substituted or unsubstituted linear or branched alkenyl group having 2 to 4 carbon atoms, and a substituted or unsubstituted linear or branched alkynyl group having 2 to 4 carbon atoms,
R² represents any one group selected from hydrogen, halogen, a hydroxyl group, a substituted or unsubstituted linear or branched alkyl group having 1 to 10 carbon atoms, a substituted or unsubstituted linear or branched alkenyl group having 2 to 10 carbon atoms, a substituted or unsubstituted linear or branched alkynyl group having 2 to 10 carbon atoms, a substituted or unsubstituted alicyclic group having 3 to 10 carbon atoms, a substituted or unsubstituted aryl group having 6 to 10 carbon atoms, a substituted or unsubstituted alkoxy group having 1 to 10 carbon atoms, a substituted or unsubstituted acyl group having 1 to 11 carbon atoms, a carboxyl group and an ester derivative or an amide derivative thereof, a substituted or unsubstituted sulfonyl group having 1 to 10 carbon atoms, and a substituted or unsubstituted sulfide group having 1 to 10 carbon atoms,
R³ represents any one group selected from hydrogen, halogen, a substituted or unsubstituted linear or branched alkyl group having 1 to 4 carbon atoms, a substituted or unsubstituted linear or branched alkenyl group having 2 to 4 carbon atoms, a substituted or unsubstituted linear or branched alkynyl group having 2 to 4 carbon atoms, a substituted or unsubstituted aryl group having 6 to 10 carbon atoms, and a substituted or unsubstituted linear or branched alkoxy group having 1 to 4 carbon atoms,
R⁴ represents any one group selected from hydrogen, a substituted or unsubstituted aryl group having 6 to 10 carbon atoms, and a substituted or unsubstituted aryloxy group having 6 to 10 carbon atoms, or optionally forms a carbonyl group together with R⁵ and carbon bonded thereto,
R⁵ represents hydrogen or optionally forms a carbonyl group together with R⁴ and carbon bonded thereto,
R⁶ represents any one group selected from hydrogen, halogen, a substituted or unsubstituted linear or branched alkyl group having 1 to 6 carbon atoms, a substituted or unsubstituted linear or branched alkenyl group having 2 to 6 carbon atoms, a substituted or unsubstituted linear or branched alkynyl group having 2 to 6 carbon atoms, a substituted or unsubstituted alicyclic group having 3 to 6 carbon atoms, a substituted or unsubstituted linear or branched alkyloxycarbonyl group having 1 to 4 carbon atoms, a substituted or unsubstituted linear or branched alkenyloxycarbonyl group having 2 to 4 carbon atoms, a substituted or unsubstituted linear or branched alkynyloxycarbonyl group having 2 to 4 carbon atoms, a substituted or unsubstituted aryl group having 6 to 10 carbon atoms, a carboxyl group and an ester derivative or an amide derivative thereof, a cyano group, and an amino group,
R⁷ represents any one group selected from hydrogen, a substituted or unsubstituted linear or branched alkyl group having 1 to 4 carbon atoms, a substituted or unsubstituted linear or branched alkenyl group having 2 to 4 carbon atoms, and a substituted or unsubstituted linear or branched alkynyl group having 2 to 4 carbon atoms,
R⁸ represents any one group selected from hydrogen, halogen, a substituted or unsubstituted linear or branched alkyl group having 1 to 6 carbon atoms, a substituted or unsubstituted linear or branched alkenyl group having 2 to 6 carbon atoms, a substituted or unsubstituted linear or branched alkynyl group having 2 to 6 carbon atoms, a substituted or unsubstituted alicyclic group having 3 to 6 carbon atoms, a substituted or unsubstituted linear or branched alkyloxycarbonyl group having 1 to 4 carbon atoms, a substituted or unsubstituted linear or branched alkenyloxycarbonyl group having 2 to 4 carbon atoms, a substituted or unsubstituted linear or branched alkynyloxycarbonyl group having 2 to 4 carbon atoms, a substituted or unsubstituted aryl group having 6 to 10 carbon atoms, a carboxyl group and an ester derivative or an amide derivative thereof, a cyano group, and an amino group, or optionally forms a carbonyl group together with R⁹ and carbon bonded thereto,
R⁹ represents hydrogen, or optionally forms a carbonyl group together with R⁸ and carbon bonded thereto,
R¹⁰ represents any one group selected from hydrogen, a substituted or unsubstituted linear or branched alkyl group having 1 to 6 carbon atoms, a substituted or unsubstituted linear or branched alkenyl group having 2 to 6 carbon atoms, a substituted or unsubstituted linear or branched alkynyl group having 2 to 6 carbon atoms, a substituted or unsubstituted alicyclic group having 3 to 6 carbon atoms, a substituted or unsubstituted linear or branched alkyloxycarbonyl group having 1 to 6 carbon atoms, a substituted or unsubstituted linear or branched alkenyloxycarbonyl group having 2 to 6 carbon atoms, a substituted or unsubstituted linear or branched alkynyloxycarbonyl group having 2 to 6 carbon atoms, a substituted or unsubstituted aryl group having 5 to 10 carbon atoms, a carboxyl group and an ester derivative or an amide derivative thereof, a cyano group, and an amino group,
R¹¹ represents any one group selected from hydrogen, a substituted or unsubstituted linear or branched alkyl group having 1 to 6 carbon atoms, a substituted or unsubstituted linear or branched alkenyl group having 2 to 6 carbon atoms, a substituted or unsubstituted linear or branched alkynyl group having 2 to 6 carbon atoms, and a substituted or unsubstituted alicyclic group having 3 to 6 carbon atoms, and
R¹⁰ and R¹¹ optionally constitute a substituted or unsubstituted nitrogen-containing heterocyclic ring together with the nitrogen atom bonded thereto],
the following formula (II): 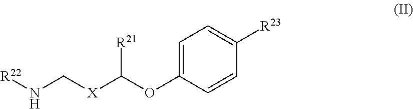
[wherein
R²¹ represents any one group selected from hydrogen, a substituted or unsubstituted aryl group having 6 to 10 carbon atoms, and a substituted or unsubstituted deuterated aryl group having 6 to 10 carbon atoms,
R²² represents any one group selected from a substituted or unsubstituted linear or branched alkyl group having 1 to 4 carbon atoms, a substituted or unsubstituted linear or branched deuterated alkyl group having 1 to 4 carbon atoms, a substituted or unsubstituted linear or branched alkenyl group having 2 to 4 carbon atoms, a substituted or unsubstituted linear or branched deuterated alkenyl group having 2 to 4 carbon atoms, a substituted or unsubstituted linear or branched alkynyl group having 2 to 4 carbon atoms, and a substituted or unsubstituted linear or branched deuterated alkynyl group having 2 to 4 carbon atoms,
R²³ represents any one group selected from hydrogen, a substituted or unsubstituted linear or branched alkyl group having 1 to 4 carbon atoms, a substituted or unsubstituted linear or branched deuterated alkyl group having 1 to 4 carbon atoms, a substituted or unsubstituted linear or branched alkenyl group having 2 to 4 carbon atoms, a substituted or unsubstituted linear or branched deuterated alkenyl group having 2 to 4 carbon atoms, a substituted or unsubstituted linear or branched alkynyl group having 2 to 4 carbon atoms, and a substituted or unsubstituted linear or branched deuterated alkynyl group having 2 to 4 carbon atoms, and
X represents methylene or deuterated methylene], and
the following formula (III): 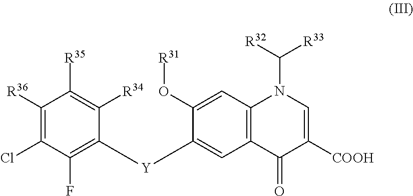
[wherein
R³¹ represents any one group selected from a substituted or unsubstituted linear or branched alkyl group having 1 to 4 carbon atoms, a substituted or unsubstituted linear or branched deuterated alkyl group having 1 to 4 carbon atoms, a substituted or unsubstituted linear or branched alkenyl group having 2 to 4 carbon atoms, a substituted or unsubstituted linear or branched deuterated alkenyl group having 2 to 4 carbon atoms, a substituted or unsubstituted linear or branched alkynyl group having 2 to 4 carbon atoms, a substituted or unsubstituted linear or branched deuterated alkynyl group having 2 to 4 carbon atoms, a substituted or unsubstituted heterocyclic group having a 5- or 6-membered ring, a substituted or unsubstituted aromatic group having 6 to 10 carbon atoms, a substituted or unsubstituted nitrogen-containing aromatic group having 2 to 10 carbon atoms, a sulfonic acid group, and a sulfonyl group,
R³² represents any one group selected from hydrogen, a hydroxymethyl group, and a hydroxymethyl group in which hydrogen on carbon is substituted by deuterium,
R³³ represents any one group selected from a substituted or unsubstituted linear or branched alkyl group having 1 to 6 carbon atoms, a substituted or unsubstituted linear or branched deuterated alkyl group having 1 to 6 carbon atoms, a substituted or unsubstituted linear or branched alkenyl group having 2 to 6 carbon atoms, a substituted or unsubstituted linear or branched deuterated alkenyl group having 2 to 6 carbon atoms, a substituted or unsubstituted linear or branched alkynyl group having 2 to 6 carbon atoms, a substituted or unsubstituted linear or branched deuterated alkynyl group having 2 to 6 carbon atoms, and a substituted or unsubstituted alicyclic group having 3 to 6 carbon atoms,
R³⁴ represents hydrogen, a hydroxymethyl group, or a hydroxymethyl group in which hydrogen on carbon is substituted by deuterium,
R³⁵ represents any one group selected from hydrogen, a hydroxyl group, a substituted or unsubstituted nitrogen-containing heterocyclic group having a 5- or 6-membered ring, and a substituted or unsubstituted nitrogen-containing aromatic group having 2 to 10 carbon atoms,
R³⁶ represents any one group selected from hydrogen, a hydroxyl group, and halogen, and
Y represents any one selected from methylene, deuterated methylene, and hydroxymethylene]
and pharmaceutically acceptable salts thereof.

2. The method for treating type 2 diabetes according to claim 1, wherein the compound is selected from the compounds represented by the following formulas (I-1) to (I-6), (II-1), (II-2), and (III-1), 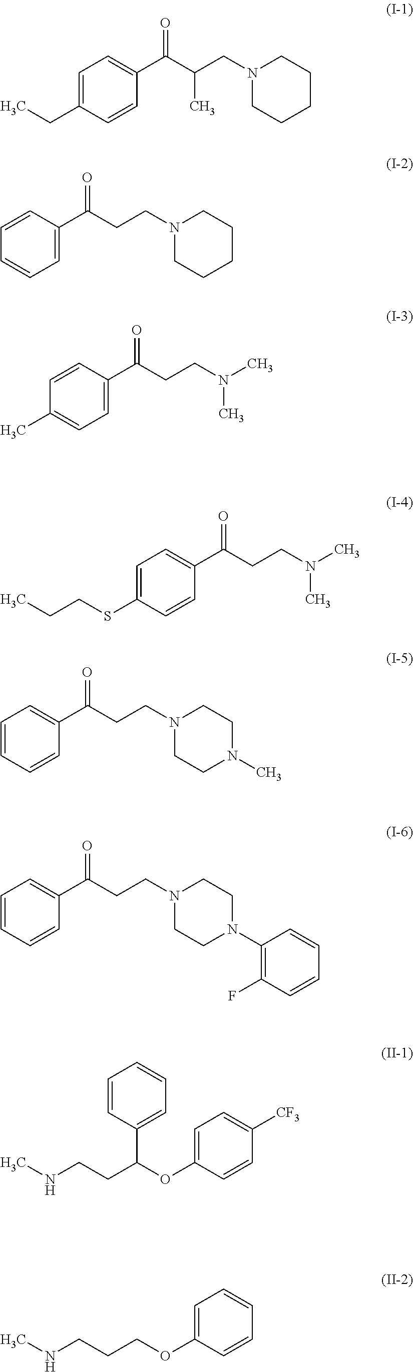 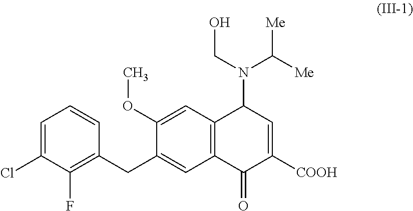 [SMILES: COc1cc2c(cc1Cc1cccc(Cl)c1F)C(=O)C(C(=O)O)=CC2N(CO)C(C)C]
and pharmaceutically acceptable salts thereof.

3. The method for treating type 2 diabetes according to claim 1, wherein the pharmaceutically acceptable salt is a salt with an acid selected from hydrochloric acid, nitric acid, sulfuric acid, sulfonic acid having 1 to 10 carbon atoms, a substituted or unsubstituted alkylcarboxylic acid having 1 to 6 carbon atoms, and a substituted or unsubstituted dicarboxylic acid having 4 to 8 carbon atoms.

4. The method for treating type 2 diabetes according to claim 3, wherein the pharmaceutically acceptable salt is a salt with an acid selected from hydrochloric acid, nitric acid, methanesulfonic acid, acetic acid, levulinic acid, lactic acid, flurbiprofen, ketoprofen, oxalic acid, fumaric acid, and maleic acid.

5. The method for treating type 2 diabetes according to claim 1, wherein the type 2 diabetes is type 2 diabetes with a reduced ability to secrete insulin caused by Cdkal1 gene mutation.

6. The method for treating type 2 diabetes according to claim 1, wherein the compound activates the conversion of proinsulin to insulin.

7. A compound represented by the following formula (IV): 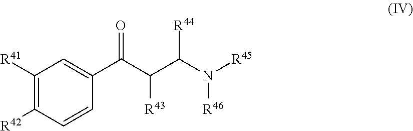
[wherein
R⁴¹ represents any one group selected from hydrogen, halogen, a hydroxyl group, a substituted or unsubstituted linear or branched alkyl group having 1 to 4 carbon atoms, a substituted or unsubstituted linear or branched alkenyl group having 2 to 4 carbon atoms, a substituted or unsubstituted linear or branched alkynyl group having 2 to 4 carbon atoms, and a substituted or unsubstituted linear or branched alkoxy group having 1 to 4 carbon atoms,
R⁴² represents any one group selected from hydrogen, halogen, a hydroxyl group, a substituted or unsubstituted linear or branched alkyl group having 1 to 4 carbon atoms, a substituted or unsubstituted linear or branched alkenyl group having 2 to 4 carbon atoms, a substituted or unsubstituted linear or branched alkynyl group having 2 to 4 carbon atoms, and a substituted or unsubstituted linear or branched alkoxy group having 1 to 4 carbon atoms,
R⁴³ represents any one group selected from hydrogen, halogen, a hydroxyl group, a substituted or unsubstituted linear or branched alkyl group having 1 to 4 carbon atoms, a substituted or unsubstituted linear or branched alkenyl group having 2 to 4 carbon atoms, a substituted or unsubstituted linear or branched alkynyl group having 2 to 4 carbon atoms, and a substituted or unsubstituted linear or branched alkoxy group having 1 to 4 carbon atoms,
R⁴⁴ represents any one group selected from hydrogen, a carboxyl group, a substituted or unsubstituted linear or branched alkyl group having 1 to 4 carbon atoms, and a substituted or unsubstituted aryl group having 6 to 10 carbon atoms,
R⁴⁵ represents any one group selected from hydrogen, a substituted or unsubstituted linear or branched alkyl group having 1 to 4 carbon atoms, a substituted or unsubstituted linear or branched alkenyl group having 2 to 4 carbon atoms, and a substituted or unsubstituted linear or branched alkynyl group having 2 to 4 carbon atoms,
R⁴⁶ represents any one group selected from hydrogen, a substituted or unsubstituted linear or branched alkyl group having 1 to 4 carbon atoms, a substituted or unsubstituted linear or branched alkenyl group having 2 to 4 carbon atoms, a substituted or unsubstituted linear or branched alkynyl group having 2 to 4 carbon atoms, a substituted or unsubstituted aryl group having 5 to 10 carbon atoms, and an arylalkyl group consisting of a substituted or unsubstituted linear or branched alkyl group having 1 to 4 carbon atoms and a substituted or unsubstituted aryl group having 5 to 10 carbon atoms, and
R⁴⁵ and R⁴⁶ optionally constitute a substituted or unsubstituted nitrogen-containing heterocyclic ring together with the nitrogen atom bonded thereto].

8. The compound according to claim 7, wherein the compound represented by the formula (IV) is represented by any of the following formulas (IV-1) to (IV-4):

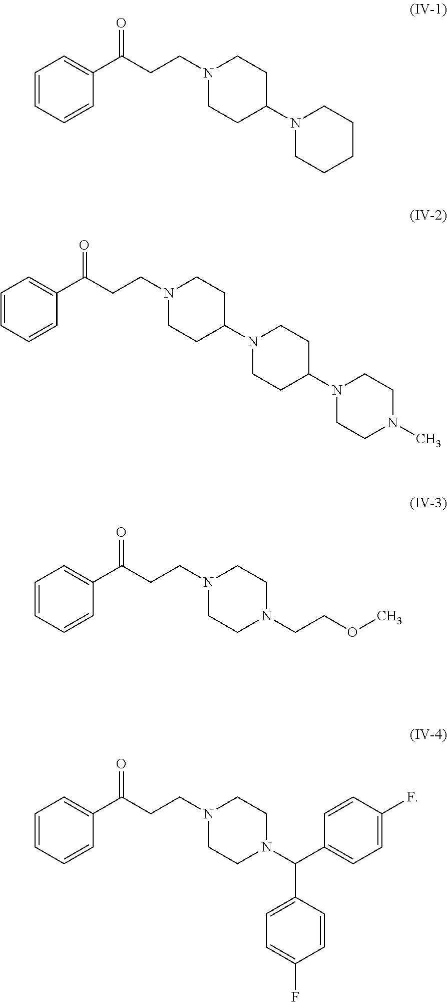 [SMILES: O=C(CCN1CCC(N2CCCCC2)CC1)c1ccccc1 ; CN1CCN(C2CCN(C3CCN(CCC(=O)c4ccccc4)CC3)CC2)CC1 ; COCCN1CCN(CCC(=O)c2ccccc2)CC1 ; O=C(CCN1CCN(C(c2ccc(F)cc2)c2ccc(F)cc2)CC1)c1ccccc1]

9. The method for treating type 2 diabetes according to claim 2, wherein the pharmaceutically acceptable salt is a salt with an acid selected from hydrochloric acid, nitric acid, sulfuric acid, sulfonic acid having 1 to 10 carbon atoms, a substituted or unsubstituted alkylcarboxylic acid having 1 to 6 carbon atoms, and a substituted or unsubstituted dicarboxylic acid having 4 to 8 carbon atoms.

10. The method for treating type 2 diabetes according to claim 2, wherein the type 2 diabetes is type 2 diabetes with a reduced ability to secrete insulin caused by Cdkal1 gene mutation.

11. The method for treating type 2 diabetes according to claim 3, wherein the type 2 diabetes is type 2 diabetes with a reduced ability to secrete insulin caused by Cdkal1 gene mutation.

12. The method for treating type 2 diabetes according to claim 4, wherein the type 2 diabetes is type 2 diabetes with a reduced ability to secrete insulin caused by Cdkal1 gene mutation.

13. The method for treating type 2 diabetes according to claim 9, wherein the type 2 diabetes is type 2 diabetes with a reduced ability to secrete insulin caused by Cdkal1 gene mutation.

14. The method for treating type 2 diabetes according to claim 2, wherein the compound activates the conversion of proinsulin to insulin.

15. The method for treating type 2 diabetes according to claim 3, wherein the compound activates the conversion of proinsulin to insulin.

16. The method for treating type 2 diabetes according to claim 4, wherein the compound activates the conversion of proinsulin to insulin.

17. The method for treating type 2 diabetes according to claim 5, wherein the compound activates the conversion of proinsulin to insulin.

18. The method for treating type 2 diabetes according to claim 9, wherein the compound activates the conversion of proinsulin to insulin.

19. The method for treating type 2 diabetes according to claim 10, wherein the compound activates the conversion of proinsulin to insulin.

20. The method for treating type 2 diabetes according to claim 11, wherein the compound activates the conversion of proinsulin to insulin.
